(19) United States
(12) Patent Application Publication
(10) Pub. No.: US 2010/0305036 A1
(43) Pub. Date: Dec. 2, 2010

(54) COMBINATION OF PROTEIN TYROSINE PHOSPHATASE INHIBITORS AND HUMAN GROWTH HORMONE FOR THE TREATMENT OF MUSCLE ATROPHY AND RELATED DISORDERS

(76) Inventors: David Barnes, Cambridge, MA (US); Angelika Christina Paul, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/744,813

(22) PCT Filed: Dec. 1, 2008
(86) PCT No.: PCT/EP08/66554

§ 371 Date: May 26, 2010

Related U.S. Application Data
(60) Provisional application No. 60/991,457, filed on Nov. 30, 2007.

(51) Int. Cl. A61K 38/27; A61K 31/433; A61P 21/00

(52) U.S. Cl. 514/11.4; 514/362

(57) ABSTRACT

The invention relates to the use of a combination of protein tyrosine phosphatase inhibitors and human growth hormone for the treatment of musculoskeletal diseases, particularly for the treatment of muscle atrophy.

The invention relates to compounds that inhibit protein tyrosine phosphatase (PTP), particularly PTP-1B, in combination with human growth hormone and the combinations use in the treatment of musculoskeletal disease.

Cellular signal transduction is a fundamental mechanism whereby external stimuli that regulate diverse cellular processes are relayed to the interior of cells. One of the key biochemical mechanisms of signal transduction involves the reversible phosphorylation of proteins, which enables regulation of the activity of mature proteins by altering their structure and function. The best characterized protein kinases in eukaryotes phosphorylate proteins on the alcohol moiety of serine, threonine and tyrosine residues. These kinases largely fall into two groups, those specific for phosphorylating serines and threonines, and those specific for phosphorylating tyrosines.

The phosphorylation state of a given substrate is also regulated by protein phosphatases, a class of proteins responsible for removal of the phosphate group added to a given substrate by a protein kinase. The protein phosphatases can also be classified as being specific for either serine/threonine or tyrosine. The known enzymes can be divided into two groups—receptor and non-receptor type proteins. Most receptor-type protein tyrosine phosphatases (RPTPs) contain two conserved catalytic tyrosine phosphatase domains each of which encompasses a segment of 240 amino acid residues (Saito et al., Cell Growth and Diff. 2:59-65, 1991). The RPTPs can be subclassified further based upon the amino acid sequence diversity of their extracellular domains (Saito, et al., supra; Krueger, et al., Proc. Natl. Acad. Sci. USA 89:7417-7421, 1992). Alignment of primary peptide sequences of both types of known PTPs shows some sequence consensus in catalytic domains and has made it possible to identify cDNAs encoding proteins with tyrosine phosphate activity via the polymerase chain reaction (PCR).

Many kinases and phosphatases are involved in regulatory cascades wherein their substrates may include, but are not limited to, other kinases and phosphatases whose activities are regulated by their phosphorylation state. Ultimately the activity of some downstream effector is modulated by phosphorylation resulting from activation of such a pathway.

It is well established that the abnormal or inappropriate activity of tyrosine kinases and/or tyrosine phosphatases plays a role in a variety of human disorders including cell proliferative disorders such as cancer, fibrotic disorders, disorders of the immune system and metabolic disorders such as diabetes.

The invention is based on the discovery that PTP inhibitors in combination with human growth hormone can be used to treat musculoskeletal diseases or conditions, particularly muscle atrophy, in a mammal such as a human individual or patient. Accordingly, the invention includes a method of treating a musculoskeletal disease by identifying an individual exhibiting the musculoskeletal disease or at risk for developing the musculoskeletal disease and administering to the individual a therapeutically effective amount of a PTP inhibitor in combination with human growth hormone sufficient to alleviate the musculoskeletal disease. The invention also includes the use of a PTP inhibitor in combination with human growth hormone in the manufacture of a medicament for the treatment or prevention of a musculoskeletal disease. In addition, the invention includes use of PTP inhibitors in combination with human growth hormone to increase muscle or bone mass in an individual, whether or not such an individual is at risk for or has a musculoskeletal disease. In general terms the invention relates to a kit comprising sequential or concurrent daily dosage units for administration each active ingredient. The combination of protein tyrosine phosphatase inhibitor compound and human growth hormone may be a fixed dosage.

In particular, the musculoskeletal disease can be muscle atrophy. There are many causes of muscle atrophy, including as a result of treatment with a glucocorticoid such as cortisol, dexamethasone, betamethasone, prednisone, methylprednisolone, or prednisolone. The muscle atrophy can also be a result of denervation due to nerve trauma or a result of degenerative, metabolic, or inflammatory neuropathy (e.g., Guillian-Barré syndrome, peripheral neuropathy, or exposure to environmental toxins or drugs). In addition, the muscle atrophy can be a result of an adult motor neuron disease, infantile spinal muscular atrophy, juvenile spinal muscular atrophy, autoimmune motor neuropathy with multifocal conductor block, paralysis due to stroke or spinal cord injury, skeletal immobilization due to trauma, prolonged bed rest, voluntary inactivity, involuntary inactivity, metabolic stress or nutritional insufficiency, cancer, AIDS, fasting, rhabdomyolysis, a thyroid gland disorder, diabetes, benign congenital hypotonia, central core disease, nemalene myopathy, myotubular (centronuclear) myopathy, burn injury, chronic obstructive pulmonary disease, liver disease, sepsis, renal failure, congestive heart failure, or ageing.

The musculoskeletal disease can also be a muscular dystrophy syndrome, such as Duchenne, Becker, myotonic, fascioscapulohumeral, Emery-Deifuss, oculopharyngeal, scapulohumeral, limb girdle, a congenital muscular dystrophy, or hereditary distal myopathy. The musculoskeletal disease can also be osteoporosis, a bone fracture, short stature, or dwarfism.

Any suitable PTP inhibitor, such as described in the next section, can be used to treat the above-mentioned musculoskeletal diseases. Since it is known that administration of human Growth Hormone has beneficial effects on the musculoskeletal system by up regulating the insulin-like growth factor pathway, human Growth Hormone can be co-administered along with the PTP inhibitor in the methods of the invention

All cited references or documents are hereby incorporated by reference.

### BRIEF DESCRIPTION OF THE DRAWING

FIG. 1 shows results from an experiment where WT and KO mice had their right leg denervated by sciatic nerve resection.

The invention relates to the treatment of musculoskeletal diseases, in particular muscle atrophy, low bone density or mineral content, and short stature. Any PTP inhibitor can be used in the methods of the invention, for example, the inhibitors described in US patents and patent application publications U.S. Pat. Nos. 7,115,624; 7,078,425; 7,022,730; 6,911,468; and 2005/0090502. In addition, specific inhibitors of PTP, particularly PTP-1B and T-cell PTP, can include the categories of compounds and specific compounds therein, as described below. Pharmaceutically acceptable human growth hormone (hGH) or a pharmaceutical equivalent is the second main component.

Listed below are definitions of various terms used to describe the compounds of the instant invention. These definitions apply to the terms as they are used throughout the specification unless they are otherwise limited in specific instances either individually or as part of a larger group. In general, whenever an alkyl group is referred to as a part of the structure, an optionally substituted alkyl is also intended.

Accordingly, the term “optionally substituted alkyl” refers to unsubstituted or substituted straight or branched chain hydrocarbon groups having 1 to 20 carbon atoms, preferably 1 to 8 carbon atoms. Exemplary unsubstituted alkyl groups include methyl, ethyl, propyl, isopropyl, n-butyl, t-butyl, isobutyl, pentyl, hexyl, isohexyl, heptyl, 4,4-dimethylpentyl, octyl and the like. Substituted alkyl groups include, but are not limited to, alkyl groups substituted by one or more of the following groups: halogen, hydroxy, cycloalkyl, cycloalkoxy, acyl, acyloxy, alkoxy, alkyloxyalkoxy, alkanoyloxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaraloxy, heterocyclyl and heterocyclyloxy including indolyl, imidazolyl, furyl, thienyl, thiazolyl, pyrrolidyl, pyridyl, pyrimidyl, piperidyl, morpholinyl and the like.

The term “lower alkyl” refers to any of the above alkyl groups as described above having 1 to 7, preferably 1 to 4 carbon atoms.

The term “halogen” or “halo” refers to fluorine, chlorine, bromine and iodine.

The term “alkenyl” refers to any of the above alkyl groups having at least 2 carbon atoms and containing a carbon to carbon double bond at the point of attachment. Groups having 2 to 8 carbon atoms are preferred.

The term “alkynyl” refers to any of the above alkyl groups having at least two carbon atoms and containing a carbon to carbon triple bond at the point of attachment. Groups having 2 to 8 carbon atoms are preferred.

The term “alkylene” refers to a straight-chain bridge of 1-6 carbon atoms connected by single bonds, e.g., —(CH2)x-, wherein x is 1-6, which may be interrupted with one or more heteroatoms selected from O, S, S(O), S(O)₂ or NR″, wherein R″ may be hydrogen, alkyl, cycloalkyl, aryl, heterocyclyl, aralkyl, heteroaralkyl, acyl, carbamoyl, sulfonyl, alkoxycarbonyl, aryloxycarbonyl or aralkoxycarbonyl and the like; and the alkylene may further be substituted with one or more substituents selected from hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C1-8)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl, heterocyclyloxy and the like.

The term “cycloalkyl” refers to optionally substituted monocyclic, bicyclic or tricyclic hydrocarbon groups of 3 to 12 carbon atoms, each of which may be substituted by one or more substituents such as alkyl, halo, oxo, hydroxy, alkoxy, alkanoyl, acylamino, carbamoyl, alkylamino, dialkylamino, thiol, alkylthio, nitro, cyano, carboxy, carboxyalkyl, alkoxycarbonyl, sulfonyl, sulfonamido, sulfamoyl, heterocyclyl and the like.

Exemplary monocyclic hydrocarbon groups include but are not limited to cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

The term “alkoxy” refers to alkyl-O—.

The term “alkanoyl” refers to alkyl-C(O)—.

The term “alkanoyloxy” refers to alkyl-C(O)—O—.

The terms “alkylamino” and “dialkylamino” refer to alkyl-NH— and (alkyl)₂N—, respectively.

The term “alkanoylamino” refers to alkyl-C(O)—NH—.

The term “alkylthio” refers to alkyl-S—.

The term “alkylaminothiocarbonyl” refers to alkyl-NHC(S)—. The term “trialkylsilyl” refers to (alkyl)₃Si—.

The term “trialkylsilyloxy” refers to (alkyl)₃SiO—.

The term “alkylthiono” refers to alkyl-S(O)—.

The term “alkylsulfonyl” refers to alkyl-S(O)₂—.

The term “alkoxycarbonyl” refers to alkyl-O—C(O)—.

The term “alkoxycarbonyloxy” refers to alkyl-O—C(O)O—.

The term “carboxycarbonyl” refers to HO—C(O)C(O)—.

The term “carbamoyl” refers to H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aralkyl-NHC(O)—, alkyl(aralkyl)-NC(O)— and the like.

The term “sulfamoyl” refers to H₂NS(O)₂—, alkyl-NHS(O)₂—, (alkyl)₂NS(O)₂—, aryl-NHS(O)₂—, alkyl(aryl)-NS(O)₂—, (aryl)₂NS(O)₂—, heteroaryl-NHS(O)₂—, aralkyl-NHS(O)₂—, heteroaralkyl-NHS(O)₂- and the like.

The term “sulfonamido” refers to alkyl-S(O)₂—NH—, aryl-S(O)₂—NH—, aralkyl-S(O)₂—NH—, heteroaryl-S(O)₂—NH—, heteroaralkyl-S(O)₂—NH—, alkyl-S(O)₂—N(alkyl)-, aryl-S(O)₂—N(alkyl)-, aralkyl-S(O)₂—N(alkyl)-, heteroaryl-S(O)₂—N(alkyl)-, heteroaralkyl-S(O)₂—N(alkyl)- and the like.

The term “sulfonyl” refers to alkylsulfonyl, arylsulfonyl, heteroarylsulfonyl, aralkylsulfonyl, heteroaralkylsulfonyl and the like.

The term “sulfonate” or “sulfonyloxy” refers to alkyl-S(O)₂—O—, aryl-S(O)₂—O—, aralkyl-S(O)₂—O—, heteroaryl-S(O)₂—O—, heteroaralkyl-S(O)₂—O— and the like.

The term “optionally substituted amino” refers to a primary or secondary amino group which may optionally be substituted by a substituent such as acyl, sulfonyl, alkoxycarbonyl, cycloalkoxycarbonyl, aryloxycarbonyl, heteroaryloxycarbonyl, aralkoxycarbonyl, heteroaralkoxycarbonyl, carboxycarbonyl, carbamoyl, alkylaminothiocarbonyl, arylaminothiocarbonyl and the like.

The term “aryl” refers to monocyclic or bicyclic aromatic hydrocarbon groups having 6 to 12 carbon atoms in the ring portion, such as phenyl, naphthyl, tetrahydronaphthyl, biphenyl and diphenyl groups, each of which may optionally be substituted by one to five substituents such as alkyl, trifluoromethyl, halo, hydroxy, alkoxy, acyl, alkanoyloxy, optionally substituted amino, thiol, alkylthio, nitro, cyano, carboxy, carboxyalkyl, alkoxycarbonyl, carbamoyl, alkylthiono, sulfonyl, sulfonamido, sulfonate, heterocyclyl and the like.

The term “monocyclic aryl” refers to optionally substituted phenyl as described under aryl.

The term “aralkyl” refers to an aryl group bonded directly through an alkyl group, such as benzyl.

The term “aralkanoyl” refers to aralkyl-C(O)—.

The term “aralkylthio” refers to aralkyl-S—.

The term “aralkoxy” refers to an aryl group bonded directly through an alkoxy group.

The term “arylsulfonyl” refers to aryl S(O)₂.

The term “arylthio” refers to aryl-S—.

The term “aroyl” refers to aryl-C(O)—.

The term “aroylamino” refers to aryl-C(O)—NH—.

The term “aryloxycarbonyl” refers to aryl-O—C(O)—.

The term “heterocyclyl” or “heterocyclo” refers to an optionally substituted, aromatic, or a partially or fully saturated nonaromatic cyclic group, for example, which is a 4- to 7-membered monocyclic, 7- to 12-membered bicyclic, or 10- to 15-membered tricyclic ring system, which has at least one heteroatom in at least one carbon atom containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2 oxopiperazinyl, 2 oxopiperidinyl, 2 oxopyrrolodinyl, 2 oxoazepinyl, azepinyl, 4 piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3 dioxolane and tetrahydro 1,1 dioxothienyl, 1,1,4-trioxo-1,2,5-thiadiazolidin-2-yl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, benzodiazepinyl, cinnolinyl, quinoxalinyl, indazolyl, pyrrolopyridyl, furopyridinyl (such as furo[2,3c]pyridinyl, furo[3,2b]-pyridinyl] or furo[2,3b]pyridinyl), dihydroisoindolyl, 1,3-dioxo-1,3-dihydroisoindol-2-yl, dihydroquinazolinyl (such as 3,4 dihydro-4 oxo-quinazolinyl), phthalazinyl and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “heterocyclyl” includes substituted heterocyclic groups. Substituted heterocyclic groups refer to heterocyclic groups that are substituted with 1, 2 or 3 substituents selected from the group consisting of the following:

(a) optionally substituted alkyl; (b) hydroxy (or protected hydroxy); (c) halo; (d) oxo (i.e. ═O); (e) optionally substituted amino, alkylamino or dialkylamino; (f) alkoxy; (g) cycloalkyl; (h) carboxy; (i) heterocyclooxy; (j) alkoxycarbonyl, such as unsubstituted lower alkoxycarbonyl; (k) mercapto; (l) nitro; (m) cyano; (n) sulfamoyl or sulfonamido; (o) alkylcarbonyloxy; (p) arylcarbonyloxy; (q) arylthio; (r) aryloxy; (s) alkylthio; (t) formyl; (u) carbamoyl; (v) aralkyl; and (w) aryl substituted with alkyl, cycloalkyl, alkoxy, hydroxy, amino, acylamino, alkylamino, dialkylamino or halo.

The term “heterocyclooxy” denotes a heterocyclic group bonded through an oxygen bridge.

The term “heteroaryl” refers to an aromatic heterocycle, for example monocyclic or bicyclic aryl, such as pyrrolyl, pyrazolyl, imidazolyl, oxazolyl, isoxazolyl, thiazolyl, isothiazolyl, furyl, thienyl, pyridyl, pyrazinyl, pyrimidinyl, pyridazinyl, indolyl, benzothiazolyl, benzoxazolyl, benzothienyl, quinolinyl, isoquinolinyl, benzimidazolyl, benzofuryl, and the like, optionally substituted by e.g. lower alkyl, lower alkoxy or halo.

The term “heteroarylsulfonyl” refers to heteroaryl S(O)₂.

The term “heteroaroyl” refers to heteroaryl-C(O)—.

The term “heteroaroylamino” refers to heteroaryl-C(O)NH—.

The term “heteroaralkyl” refers to a heteroaryl group bonded through an alkyl group.

The term “heteroaralkanoyl” refers to heteroaralkyl-C(O)—.

The term “heteroaralkanoylamino” refers to heteroaralkyl-C(O)NH—.

The term “acyl” refers to alkanoyl, cycloalkanoyl, aroyl, heteroaroyl, aralkanoyl, heteroaralkanoyl and the like.

The term “acyloxy” refers to alkanoyloxy, cycloalkanoyloxy, aroyloxy, heteroaroyloxy, aralkanoyloxy, heteroaralkanoyloxy and the like.

The term “acylamino” refers to alkanoylamino, cycloalkanoylamino, aroylamino, heteroaroylamino, aralkanoylamino, heteroaralkanoylamino and the like.

The term “esterified carboxy” refers to optionally substituted alkoxycarbonyl, cycloalkoxycarbonyl, aryloxycarbonyl, aralkoxycarbonyl, heterocyclooxycarbonyl and the like.

Pharmaceutically acceptable salts of any compound useful in the present invention refer to salts formed with bases, namely cationic salts such as alkali and alkaline earth metal salts, such as sodium, lithium, potassium, calcium, magnesium, as well as ammonium salts, such as ammonium, trimethylammonium, diethylammonium, and tris(hydroxymethyl)-methylammonium salts, and salts with amino acids.

Similarly acid addition salts, such as those formed with mineral acids, organic carboxylic acids and organic sulfonic acids e.g. hydrochloric acid, maleic acid and methanesulfonic acid, are possible provided a basic group, such as pyridyl, constitutes part of the structure.

In starting compounds and intermediates which are converted to the compounds useful in the invention in a manner described herein, functional groups present, such as amino, thiol, carboxyl, and hydroxy groups, are optionally protected by conventional protecting groups that are common in preparative organic chemistry. Protected amino, thiol, carboxyl, and hydroxyl groups are those that can be converted under mild conditions into free amino thiol, carboxyl and hydroxyl groups without the molecular framework being destroyed or other undesired side reactions taking place.

The purpose of introducing protecting groups is to protect the functional groups from undesired reactions with reaction components under the conditions used for carrying out a desired chemical transformation. The need and choice of protecting groups for a particular reaction is known to those skilled in the art and depends on the nature of the functional group to be protected (hydroxyl group, amino group, etc.), the structure and stability of the molecule of which the substituent is a part and the reaction conditions. Well known protecting groups that meet these conditions and their introduction and removal are described, for example, in McOmie, “Protective Groups in Organic Chemistry”, Plenum Press, London, New York (1973); and Greene and Wuts, “Protective Groups in Organic Synthesis”, John Wiley and Sons, Inc, New York (1999).

The above mentioned reactions are carried out according to standard methods, in the presence or absence of diluent, preferably such as are inert to the reagents and are solvents thereof, of catalysts, condensing or said other agents respectively and/or inert atmospheres, at low temperatures, room temperature or elevated temperatures (preferably at or near the boiling point of the solvents used), and at atmospheric or super-atmospheric pressure.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds useful in the invention and intermediates can also be converted into each other according to methods generally known per se.

Depending on the choice of starting materials and methods, the compounds may be in the form of one of the possible isomers or mixtures thereof, for example, as substantially pure geometric (cis or trans) isomers, optical isomers (enantiomers, antipodes), racemates, or mixtures thereof. The aforesaid possible isomers or mixtures thereof are within the purview of this invention.

Any resulting mixtures of isomers can be separated on the basis of the physico-chemical differences of the constituents, into the pure geometric or optical isomers, diastereoisomers, racemates, for example by chromatography and/or fractional crystallization.

Any resulting racemates of final products or intermediates can be resolved into the optical antipodes by known methods, e.g. by separation of the diastereoisomeric salts thereof, obtained with an optically active acid or base, and liberating the optically active acidic or basic compound. The carboxylic acid intermediates can thus be resolved into their optical antipodes e.g. by fractional crystallization of D- or L-(alpha-methylbenzylamine, cinchonidine, cinchonine, quinine, quinidine, ephedrine, dehydroabietylamine, brucine or strychnine)-salts. Racemic products can also be resolved by chiral chromatography, e.g. high pressure liquid chromatography using a chiral adsorbent.

Finally, compounds useful in the invention are either obtained in the free form, as a salt thereof if salt forming groups are present or as prodrug derivatives thereof. In particular, the NH-group of the 1,1-dioxo-1,2,5-thiadiazolidin-3-one moiety, may be converted into salts with pharmaceutically acceptable bases. Salts may be formed using conventional methods, advantageously in the presence of an ethereal or alcoholic solvent, such as a lower alkanol. From the solutions of the latter, the salts may be precipitated with ethers, e.g. diethyl ether. Resulting salts may be converted into the free compounds by treatment with acids. These or other salts can also be used for purification of the compounds obtained.

Compounds useful in the invention having basic groups can be converted into acid addition salts, especially pharmaceutically acceptable salts. These are formed, for example, with inorganic acids, such as mineral acids, for example sulfuric acid, a phosphoric or hydrohalic acid, or with organic carboxylic acids, such as (C₁₋₄)alkanecarboxylic acids which, for example, are unsubstituted or substituted by halogen, for example acetic acid, such as saturated or unsaturated dicarboxylic acids, for example oxalic, succinic, maleic or fumaric acid, such as hydroxy-carboxylic acids, for example glycolic, lactic, malic, tartaric or citric acid, such as amino acids, for example aspartic or glutamic acid, or with organic sulfonic acids, such as (C₁₋₄)alkyl-sulfonic acids (for example methanesulfonic acid) or arylsulfonic acids which are unsubstituted or substituted (for example by halogen). Preferred are salts formed with hydrochloric acid, methanesulfonic acid and maleic acid.

Prodrug derivatives of any compound of the present invention are derivatives of said compounds which following administration release the parent compound in vivo via some chemical or physiological process, e.g., a prodrug on being brought to the physiological pH or through enzyme action is converted to the parent compound. Exemplary prodrug derivatives are, e.g., esters of free carboxylic acids and S-acyl and O-acyl derivatives of thiols, alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art.

In view of the close relationship between the free compounds, the prodrug derivatives and the compounds in the form of their salts, whenever a compound is referred to in this context, a prodrug derivative and a corresponding salt is also intended, provided such is possible or appropriate under the circumstances.

The compounds, including their salts, can also be obtained in the form of their hydrates, or include other solvents used for their crystallization.

Thus, the pharmacologically active compounds useful in the invention may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

The pharmaceutical compositions useful in the invention may contain a therapeutically effective amount of a compound as defined above with human growth hormone, in a combination with another therapeutic agent, e.g., each at an effective therapeutic dose as reported in the art. One such compound that can be administered along with the PTP inhibitor is human insulin-like growth factor1 or IGF1, however formulated or stabilized, such as INCRELEX™ as developed by Tercica or as described in US 2006/0166328.

The structure of the therapeutic agents identified by code numbers, generic or trade names may be taken from the actual edition of the standard compendium “The Merck Index” or from databases, e.g., Patents International (e.g. IMS World Publications).

As used throughout the specification and in the claims, the term “treatment” embraces all the different forms or modes of treatment as known to those of the pertinent art and in particular includes preventive, curative, delay of progression and palliative treatment.

The above-cited properties are demonstrable in vitro and in vivo tests, using advantageously mammals, e.g., mice, rats, dogs, monkeys or isolated organs, tissues and preparations thereof. Said compounds can be applied in vitro in the form of solutions, e.g. preferably aqueous solutions, and in vivo either enterally, parenterally, advantageously intravenously, e.g. as a suspension or in aqueous solution. The dosage in vitro may range between about 10⁻³ molar and 10⁻¹⁰ molar concentrations. A therapeutically effective amount in vivo may range depending on the route of administration, between about 1 and 500 mg/kg, preferably between about 5 and 100 mg/kg.

The activity of a compound according to the invention may be assessed by the following methods or by following methods well described in the art (e.g. Peters G. et al. J. Biol. Chem., 2000, 275, 18201-09).

For example, the PTP-1B inhibitory activity in vitro may be determined as follows:

Assessment of human PTP-1B (hPTP-1B) activity in the presence of various agents is determined by measuring the amount of inorganic phosphate released from a phosphopeptide substrate using a 96-well microtiter plate format. The assay (100 μL) is performed in an assay buffer comprised of 50 mM TRIS (pH 7.5), 50 mM NaCl, 3 mM DTT at ambient temperature. The assay is typically performed in the presence of 0.4% dimethyl sulfoxide (DMSO). However, concentrations as high as 10% are used with certain poorly soluble compounds. A typical reaction is initiated by the addition of 0.4 pmoles of hPTP-1B (amino acids 1-411) to wells containing assay buffer, 3 nmoles of the synthetic phosphopeptide substrate (GNGDpYMPMSPKS), and the test compound. After 10 min, 180 μL malachite green reagent (0.88 mM malachite green, 8.2 mM ammonium molybdate, aqueous 1 N HCl, and 0.01% Triton X-100) is added to terminate the reaction. Inorganic phosphate, a product of the enzyme reaction, is quantitated after 15 min as the green color resulting from complexing with the Malichite reagent and is determined as an A₆₂₀ using a Molecular Devices (Sunnyvale, Calif.) SpectraMAX Plus spectrophotometer. Test compounds are solubilized in 100% DMSO (Sigma, D-8779) and diluted in DMSO. Activity is defined as the net change in absorbance resulting from the activity of the uninhibited hPTP-1B_{[1-411]} minus that of a tube with acid-inactivated hPTP-1B_{[1-411]}.

The hPTP-1B_{[1-411]} is cloned by PCR from a human hippocampal cDNA library (Clonetech) and inserted into a pET 19-b vector (Novagen) at the Nco1 restriction site. E. coli strain BL21 (DE3) is transformed with this clone and stored as a stock culture in 20% glycerol at −80° C. For enzyme production, a stock culture is inoculated into LB/Amp and grown at 37° C. Expression of PTP-1B is initiated by induction with 1 mM IPTG after the culture had reached an OD₆₀₀=0.6. After 4 h, the bacterial pellet is collected by centrifugation. Cells are resuspended in 70 mL lysis buffer (50 mM Tris, 100 mM NaCl, 5 mM DTT, 0.1% Triton X-100, pH7.6), incubated on ice for 30 min then sonicated (4×10 sec bursts at full power). The lysate is centrifuged at 100,000×g for 60 min and the supernatant is buffer exchanged and purified on a cation exchange POROS 20SP column followed by an anion exchange Source 30Q (Pharmacia) column, using linear NaCl gradient elutions. Enzyme is pooled, adjusted to 1 mg/mL and frozen at −80° C.

Alternatively, the assessment of human PTP-1B activity in the presence of various agents may be determined by measuring the hydrolysis products of known competing substrates. For example, cleavage of substrate para-nitrophenylphosphate (pNPP) results in the release of the yellow-colored para-nitrophenol (pNP) which can be monitored in real time using a spectrophotometer. Likewise, the hydrolysis of the fluorogenic substrate 6,8-difluoro-4-methylumbelliferyl phosphate ammonium salt (DiFMUP) results in the release of the fluorescent DiFMU which can be readily followed in a continuous mode with a fluorescence reader (Anal. Biochem. 273, 41, 1999; Anal. Biochem. 338, 32, 2005):

pNPP Assay

Compounds are incubated with 1 nM recombinant human PTP-1B_{[1-298]} or PTP-1B_{[1-322]} in buffer (50 mM Hepes, pH 7.0, 50 mM KCl, 1 mM EDTA, 3 mM DTT, 0.05% NP-40 for 5 min at room temperature. The reaction is initiated by the addition of pNPP (2 mM final concentration) and run for 120 min at room temperature. Reactions are quenched with 5 N NaOH. Absorbance at 405 nm is measured using any standard 384 well plate reader.

### DiFMUP Assay

Compounds are incubated with 1 nM recombinant human PTP-1B_{[1-298]} or PTP-1B_{[1-322]} in buffer (50 mM Hepes, pH 7.0, 50 mM KCl, 1 mM EDTA, 3 mM DTT, 0.05% NP-40 (or 0.001% BSA) for 5 min at room temperature. The reaction is initiated by the addition of DiFMUP (6 μM final concentration) and run kinetically on fluorescence plate reader at 355 nm excitation and 460 nm emission wavelengths. Reaction rates over 15 min are used to calculate inhibition.

PTP-1B_{[1-298]} is expressed in E. coli BL21(DE3) containing plasmids constructed using pET19b vectors (Novagen). The bacteria are grown in minimal media using an “On Demand” Fed-batch strategy. Typically, a 5.5 liter fermentation is initiated in Fed-batch mode and grown overnight unattended at 37° C. Optical densities varied between 20-24 OD₆₀₀ and the cultures are induced at 30° C. with IPTG to a final concentration of 0.5 mM. The bacterial cells are harvested 8 hours later and yield 200-350 gm (wet weight). The cells are frozen as pellets and stored at −80° C. until use. All steps are performed at 4° C. unless noted. Cells (−15 g) are thawed briefly at 37° C. and resuspended in 50 mL of lysis buffer containing 50 mM Tris-HCl, 150 mM NaCl, 5 mM DTT, pH 8.0 containing one tablet of Complete (EDTA-free) protease cocktail (Boehringer Mannheim), 100 μM PMSF and 100 μg/mL DNase I. The cells are lysed by sonication (4×10 second burst, full power) using a Virsonic 60 (Virtus). The pellet is collected at 35,000×g, resuspended in 25 mL of lysis buffer using a Polytron and collected as before. The two supernatants are combined and centrifuged for 30 min at 100,000×g. The soluble lysate could be stored at this stage at −80° C. or used for further purification. Diafiltration using a 10 kD MWCO membrane is used to buffer exchange the protein and reduce the NaCl concentration prior to cation exchange chromatography. Diafiltration buffer contained 50 mM MES, 75 mM NaCl, 5 mM DTT, pH 6.5. Soluble supernatant is then loaded onto a POROS 20 SP (1×10 cm) column equilibrated with cation exchange buffer (50 mM MES and 75 mM NaCl, pH 6.5) at a rate of 20 ml/min. An analytical column (4.6×100 mm) is run in a similar fashion except the flow rate was reduced to 10 mL/min. Protein is eluted from the column using a linear salt gradient (75-500 mM NaCl in 25 CV). Fractions containing PTP-1B_{[1-298]} are identified and pooled according to SDS-PAGE analyses. Final purification is performed using Sephacryl S-100 HR (Pharmacia). The column (2.6×35 cm) is equilibrated with 50 mM HEPES, 100 mM NaCl, 3 mM DTT, pH 7.5 and run at a flow rate of 2 mL/min. The final protein is pooled and concentrated to ˜5 mg/mL using an Ultrafree-15 concentrator (Millipore) with a MWCO 10,000. The concentrated protein is stored at −80° C. until use.

Competitive binding to the active site of the enzyme can be determined as follows: Ligand binding is detected by acquiring ¹H-¹⁵N HSQC spectra on 250 μL of 0.15 mM PTP-1B_{[1-298]} in the presence and absence of added compound (1-2 mM). The binding is determined by the observation of ¹⁵N- or ¹H-amide chemical shift changes in two dimensional HSQC spectra upon the addition of a compound to ¹⁵N-label protein. Because of the ¹⁵N spectral editing, no signal from the ligand is observed, only protein signals. Thus, binding can be detected at high compound concentrations. Compounds which caused a pattern of chemical shift changes similar to the changes seen with known active site binders are considered positive.

All proteins are expressed in E. coli BL21 (DE3) containing plasmids constructed using pET19b vectors (Novagen). Uniformly ¹⁵N-labeled PTP-1B₁₋₂₉₈ is produced by growth of bacteria on minimal media containing ¹⁵N-labeled ammonium chloride. All purification steps are performed at 4° C. Cells (−15 g) are thawed briefly at 37° C. and resuspended in 50 mL of lysis buffer containing 50 mM Tris-HCl, 150 mM NaCl, 5 mM DTT, pH 8.0 containing one tablet of Complete (EDTA-free) protease cocktail (Boehringer Mannheim), 100 μM PMSF and 100 μg/mL DNase I. The cells are lysed by sonication. The pellet is collected at 35,000×g, resuspended in 25 mL of lysis buffer using a Polytron and collected as before. The two supernatants are combined and centrifuged for 30 min at 100,000×g. Diafiltration using a 10 kD MWCO membrane is used to buffer exchange the protein and reduce the NaCl concentration prior to cation exchange chromatography. Diafiltration buffer contained 50 mM MES, 75 mM NaCl, 5 mM DTT, pH 6.5. Soluble supernatant is then loaded onto a POROS 20 SP (1×10 cm) column equilibrated with cation exchange buffer (50 mM MES and 75 mM NaCl, pH 6.5) at a rate of 20 mL/min. Protein is eluted from the column using a linear salt gradient (75-500 mM NaCl in 25 CV). Fractions containing PTP-1B's are identified and pooled according to SDS-PAGE analyses. PTP-1B₁₋₂₉₈ is further purified by anion exchange chromatography using a POROS 20 HQ column (1×10 cm). The pool from cation exchange chromatography is concentrated and buffer exchanged in 50 mM Tris-HCl, pH 7.5 containing 75 mM NaCl and 5 mM DTT. Protein is loaded onto column at 20 mL/min and eluted using a linear NaCl gradient (75-500 mM in 25 CV). Final purification is performed using Sephacryl S-100 HR (Pharmacia) (50 mM HEPES, 100 mM NaCl, 3 mM DTT, pH 7.5). The NMR samples are composed of uniformly ¹⁵N-labeled PTP-1B₁₋₂₉₈ (0.15 mM) and inhibitor (1-2 mM) in a 10% D₂O/90% H₂O Bis-Tris-d₁₉ buffer (50 mM, pH=6.5) solution containing NaCl (50 mM), DL-1,4-Dithiothreitol-d₁₀ (5 mM) and Sodium azide (0.02%).

The ¹H-¹⁵N HSQC NMR spectra are recorded at 20° C., on Bruker DRX500 or DMX600

NMR spectrometers. In all NMR experiments, pulsed field gradients are applied to afford the suppression of solvent signal. Quadrature detection in the indirectly detected dimensions is accomplished by using the States-TPPI method. The data are processed using Bruker software and analyzed using NMRCompass software (MSI) on Silicon Graphics computers.

### Analytical Methods for Measuring Inhibitor Activity in Muscle Disease Models

To determine if a PTP inhibitor is useful for the treatment of musculoskeletal disease, particularly muscle atrophy, the following in vitro and animal model assays can be used.

### Tissue Culture

C2C12 cells can be obtained from the American Type Tissue Culture Bank and propagated in standard growth media containing 10% horse serum. When cells reached 70% confluency the media is changed to differentiation media containing 2% horse serum. Three days after start of differentiation multinucleated myotubes should be present, with visible striations, indicating differentiation. hGH can be used as a positive control for activating the IGF-1 receptor, or alternatively, combined with a PTP1B inhibitor to assess synergistic or additive effects. Consequently, PTP1B inhibitors and/or hGH can be added to the media for various times.

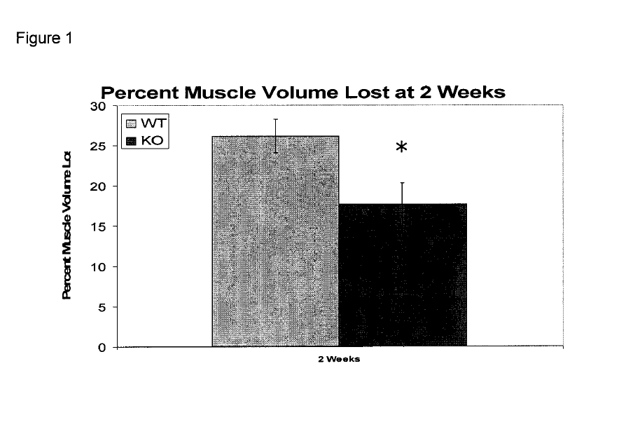

Myotube Diameter Myotube diameter can be assessed 24 hours after addition of PTP1B inhibitor and/or hGH to myotube media. Cells are washed in saline and fixed in gluteraldehyde. Images can be obtained on an inverted microscope using the green fluorescent channel to visualize the auto-fluorescence induced by the gluteraldehyde fixation. Images are printed out at standard magnification and the sixty largest myotubes measured at their largest diameter (as previously published). The fifty largest myotubes in each group can be statistically compared using the Kruskal-Wallis One Way Analysis on Ranks. Differences are considered significant if p<0.05 and designated by an asterisk in FIG. 1.

pAKT assay Phosphorylation levels of AKT, a protein that is phosphorylated when the IGF1 protein binds to its receptor, can be assessed using a commercially available pAKT ELISA kit (Cell Signaling, Pathscan 7160) and the corresponding total AKT ELISA kit (Cell Signaling, Pathscan 7170), following manufacturer instructions.

pIGFR assay Phosphorylation levels of the IGF receptor in cell cultures can be assessed by lysis of the cells, immunoprecipitation of the IGFR using anti-IGFR antibodies (Transduction Laboratories, Lexington, Ky., USA), Western blot analysis using anti-phosphotyrosine antibodies (Upstate Biotechnology, USA). See, e.g., Shefi-Friedman et al., Am J Physiol Endocrinol Metab 281:E16-E24, 2001.

PTP1B inhibitors and hGH hGH is Human Growth Hormone and it can be purchased from Bachem H-3148 and added to C2C12 culture media or GH expression vectors can be transfected into C2C12. Multiple PTP1B inhibitors can be tested with or without hGH.

### In Vivo Testing in Mouse Exercise Models of Hypertrophy

To determine whether a PTP inhibitor can act to increase skeletal muscle mass under an exercise context that already leads to muscle hypertrophy, one can subject treated and untreated animals to exercise and determine whether animals receiving the PTP inhibitor have developed larger muscles than untreated animals.

One model known in the art is based on the use of a voluntary running wheel with user-variable loads (see, e.g., Konhilas et al., Am J Physiol Heart Circ Physiol 289:H455-H465, 2005). The voluntary cage wheel eliminates physical and psychological insults that are common in forced exercised models, and are therefore more appropriate for evaluating candidate drugs that are used in relatively healthy individuals for whom increases in muscle mass is desirable.

Any suitable mouse strain can be used. For example, male C57BI/6J mice can be randomly assigned to experimental (e.g., receiving PTP inhibitor) and control groups. Animals are individually housed in a cage containing an exercise wheel; sedentary control animals are housed in identical cages without a wheel. The exercise wheels are described in Allen et al., J Appl Physiol 90:1900-1908, 2001. Briefly, the system consists of an 11.5 cm-diameter wheel with a 5.0 cm-wide running surface (model 6208, Petsmart, Phoenix, Ariz.) equipped with a digital magnetic counter (model BC 600, Sigma Sport, Olney, Ill.) that is activated by wheel rotation. In addition, each wheel is engineered with a resistance mechanism allowing adjustment of the load. This is accomplished by attaching stainless steel fishing line to the cage top and wrapping the wire around an immovable pulley that is secured to the cage wheel at the axis of rotation so as to not contribute to the wheel load. The wire is again secured to the cage top with a spring and screw. This design permits fine adjustments of the wheel load, which is evenly distributed throughout the rotation of the wheel. Daily exercise values for time and distance run are recorded for each exercised animal throughout the duration of the exercise period. All animals are given water and standard hard rodent chow ad libitum. Voluntary running (cage wheel exposure) can begin at an average age of about 12 weeks for all groups. Each group continues running under varying resistance, depending on experimental group, for 50 days until the animals are about 19 weeks of age. The load on the wheel is determined by hanging known weights on the wheel until the wheel was slightly displaced. All exercise groups begin with no load on the cage wheel for the first week. However, the “no-load” condition is actually 2 g, which is determined as the load necessary to maintain wheel inertia and frictional load. Considering a wheel acclimatization period of 1 week, wheel loads can be changed at one-week intervals, except for higher loads, which can be changed after 2 weeks. The range of loads can be anywhere from 2 g to up to 12 g. Exercised and sedentary control animals are euthanized by cervical dislocation under inhaled anesthesia immediately after the end of the specific exercise period. Body mass is measured, and specific muscles are rapidly excised, washed, and frozen for histological or biochemical assays at a future date.

Alternative exercise hypertrophy models are also available to the skilled artisan. See, e.g., the treadmill exercise model described in Lerman et al., J Appl Physiol 92:2245-2255, 2002.

### In Vivo Testing on PTP1B Null Mutant Mice

In this in vivo model, lack of PTP1b can be tested for the ability to maintain muscle mass under conditions that generally reduce muscle mass.

Mice Heterozygous PTP1B null mice can be purchased from Deltagen (San Carlos, Calif., USA) and mated to produce offspring that are null, heterozygous, or wild-type litter mates. Mice are housed under ACUC protocol 06 MG 0144 and maintained with food and water ad libitum in a 12 hour light cycle. Mice can be genotyped by PCR on tail biopsies.

Denervation The right sciatic nerve is resected during deep anesthesia under ACUC protocol 06 MG 0189. Briefly, anesthesia is induced using isofluorane inhalation, the right leg shaved and sterilized. An incision is then made through the skin of the lateral right leg and the sciatic nerve visualized. The nerve is cut and a 0.3 to 0.5 section removed to prevent reattachment. The incision was closed with wound clips and the mice returned to their cages for recovery from anesthesia. The contralateral leg is unperturbed and serves as internal control. Mice are euthanized 14 days after denervation surgery and muscles and other tissues isolated for further processing.

Muscle and Tissue Weight The following tissues can be dissected from PTP1B knock out knock out (KO), heterozygous (HET), and wild-type (WT) mice 14 days after denervation of the right hind limb: left and right tibialis anterior muscles, left and right extensor digitorum longus muscles, left and right soleus muscles, left and right gastrocnemius muscles, heart, liver, spleen, epididimal white adipose tissue, brown adipose tissue, and blood for serum isolation. Each tissue is freed from connective tissue and weighed, before being snap frozen for further analysis to be completed.

### Experimental Set-Up and Analysis

The effect of PTP1B inhibition on muscle disease can be measured, as described above, in an in vitro model system of skeletal muscle atrophy, using the C2C12 cell line, and in an in vivo model of skeletal muscle atrophy, using PTP1B wild type, heterozygous, and homozygous null mice in combination with denervation-induced skeletal muscle atrophy.

Treatment of C2C12 myotubes with PTP1B inhibitors can result in an increase in myotube size, when the diameter of the fifty largest myotubes is measured.

When IGF1 alone is added to C2C12 myotubes media at a concentration of 10 ng/ml, myotubes can be significantly hypertrophied. Addition of hGH alone can also cause a significant increase in C2C12 myotube diameter. A PTP inhibitor can result in an increase in myotube diameter that is significantly bigger than the diameter of untreated myotubes. There might not be a statistical significance between cells treated with either IGF1, hGH, or an inhibitor individually, but when myotubes are treated with both IGF1 or hGH and an inhibitor simultaneously, this could result in a significant increase in myotube diameter when compared to the singly-treated cells. Such a result would indicate that the IGF1 pathway and a PTP inhibitor act at least additively and potentially synergistically to produce larger myotubes and therefore indicate that hGH and a PTP inhibitor can be co-administered to a mammal to increase muscle mass or to treat muscle atrophy. Alternatively, assay results can indicate that a PTP inhibitor is active alone, in which case use of the inhibitor in monotherapy to increase muscle mass or to treat muscle atrophy is indicated.

PTP1B null mice can be used to test whether absence of PTP1B can prevent or ameliorate skeletal muscle atrophy. Fourteen days after denervation of the right hind limb, muscle weight of denervated muscles is compared to contralateral muscles from the control leg (wet muscle weight is normalized to individual body weight). Using this animal model of skeletal muscle atrophy, there is significant sparing of muscles in heterozygous and knock-out mice. WT mice lose about 40% of gastrocnemius muscle mass, compared with a 30% loss in heterozygous mice and 20% in homozygous null mice. Half of the denervation-induced muscle atrophy is prevented in KO gastrocnemius muscle compared to WT gastrocnemius muscle.

Phosphorylation of AKT is a downstream event of IGF1R phosphorylation. Levels of AKT phosphorylation can be used as a read-out of activation of the IGF1 pathway in C2C12 myotubes treated with a PTP inhibitor +/−IGF1 or +/−hGH for one hour. A PTP inhibitor alone can increase phosphorylation levels of AKT by for example at least 50% (e.g., 65%). hGH treatment is known to cause AKT phosphorylation in C2C12, but treatment of hGH with a PTP inhibitor can further increases AKT phosphorylation above that induced by hGH alone. Treatment with a PTP inhibitor can therefore increase activation of the IGF1 pathway, possibly resulting in a significant increase in myotube diameter in the manner described above.

Results from experiments such as described above can indicate that PTP inhibitors, particularly PTP1B inhibitors, increase levels of pAKT, which in turn result in a physiological increase in myotube diameter. Observations can include:
A PTP inhibitor causes a significant increase in C2C12 myotube diameter by itself and acts at least additively with hGH in media to further increase myotube size.
A PTP inhibitor causes a significant increase in C2C12 myotubes when compared to untreated myotubes.

Furthermore, in vivo atrophy study using PTP1B KO and WT mice with denervation-induced muscle atrophy can show that inhibition of PTP1B prevents skeletal muscle atrophy.

Provided that a PTP inhibitor exhibits in vitro or in vivo activity for increasing muscle mass, e.g., by way of modulating the IGF1 signaling pathway at the level of AKT phosphorylation, such PTP inhibitors would then also be useful for other diseases known to be amenable to IGF1 or human Growth Hormone treatment, such as dwarfism, low bone density or mineral content, osteoporosis or short stature.

The methods of the invention can be practiced with 1,1-dioxo-1,2,5-thiadiazolidin-3-one derivatives as described in the categories below.

### Category 1 PTP Inhibitors

The methods of the present invention can be practiced with the compounds of the formula

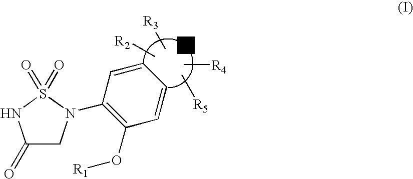

wherein
Q combined together with the carbon atoms to which it is attached form an aromatic, or a partially or fully saturated nonaromatic 5- to 8-membered carbocyclic or heterocyclic ring;
R₁ is hydrogen, —C(O)R₆, —C(O)NR₇R₈ or —C(O)OR₉ in which
   R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂, R₃, R₄ and R₅ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ and R₃ combined are alkylene which together with the ring atoms to which they are attached form a 3- to 7-membered fused ring; or
   R₂ and R₃ combined are alkylene which together with the carbon atom to which they are attached form a 3- to 7-membered spirocyclic ring;
   or a pharmaceutically acceptable salt thereof.

Preferred are compounds in the A group having the formula

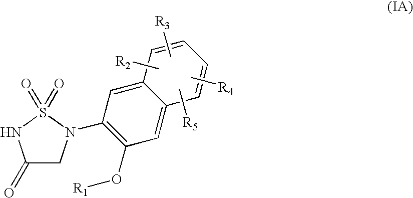

wherein
R₁ is hydrogen, —C(O)R₆, —C(O)NR₇R₈ or —C(O)OR₉ in which
   R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂, R₃, R₄ and R₅ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ and R₃ combined are alkylene which together with the ring atoms to which they are attached form a 5- to 7-membered fused ring; or
   or a pharmaceutically acceptable salt thereof.

Preferred are compounds of formula (IA) wherein
R₄ and R₅ are hydrogen;
   or a pharmaceutically acceptable salt thereof.

Further preferred are compounds of formula (IA) having the formula

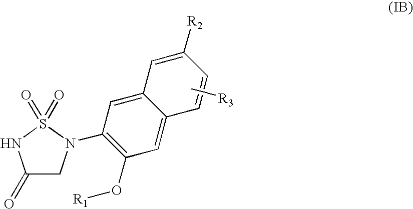

wherein
R₁ is hydrogen, —C(O)R₆, —C(O)NR₇R₈ or —C(O)OR₉ in which
   R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ and R₃ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
   or a pharmaceutically acceptable salt thereof.

Preferred are compounds of formula (IB) wherein
R₂ is —Y—(CH₂)ₙ—CR₁₀R₁₁—(CH₂)ₘ—X in which
   Y is oxygen or S(O)_{q} in which q is zero or an integer of 1 or 2; or
   Y is trans CH═CH; or
   Y is absent;
   n is an integer from 1 to 6;
   R₁₀ and R₁₁ are, independently from each other, hydrogen or lower alkyl; or
      R₁₀ and R₁₁ combined are alkylene which together with the carbon atom to which they are attached form a 3- to 7-membered ring;
m is zero or an integer of 1 or 2;
   X is hydroxy, alkoxy, cycloalkyl, cycloalkoxy, acyl, acyloxy, carbamoyl, cyano, trifluoromethyl, free or esterified carboxy, monocyclic aryl or heterocyclyl;
   or a pharmaceutically acceptable salt thereof.

Further preferred are compounds of formula (IB) wherein
R₃ is hydrogen;
   or a pharmaceutically acceptable salt thereof.

Further preferred are also compounds of formula (IB) wherein
n is an integer of 2 or 3;
R₁₀ and R₁₁ are, independently from each other, hydrogen or lower alkyl;
m is zero or 1;
   X is hydroxy, carbamoyl, cyano, trifluoromethyl, free or esterified carboxy, monocyclic aryl or heterocyclyl;
   or a pharmaceutically acceptable salt thereof.

More preferred are compounds of formula (IB) wherein
Y is absent;
   or a pharmaceutically acceptable salt thereof.

Even more preferred are compounds of formula (IB) wherein
n is 3;
R₁₀ and R₁₁ are lower alkyl;
m is zero or 1;
X is hydroxy, cyano or free or esterified carboxy;
   or a pharmaceutically acceptable salt thereof.

Most preferred are compounds of formula (IB) wherein
R₁₀ and R₁₁ are methyl;
   or a pharmaceutically acceptable salt thereof.

Especially preferred are compounds of formula (IB) wherein
R₁ is hydrogen or —C(O)R₆ in which R₆ is monocyclic aryl;
   or a pharmaceutically acceptable salt thereof.

Preferred are also compounds in the A group having the formula

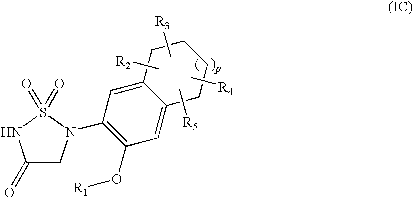

wherein
R₁ is hydrogen or —C(O)R₆, —C(O)NR₇R₆ or —C(O)OR₉ in which
   R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂, R₃, R₄ and R₅ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ and R₃ combined are alkylene which together with the ring atoms to which they are attached form a 3- to 7-membered fused ring; or
   R₂ and R₃ combined are alkylene which together with the carbon atom to which they are attached form a 3- to 7-membered spirocyclic ring;
   p is zero or 1;
   or a pharmaceutically acceptable salt thereof.

Preferred are compounds of formula (IC) wherein
R₄ and R₅ are hydrogen;
   or a pharmaceutically acceptable salt thereof.

Preferred are also compounds of formula (IC) wherein

R₂ and R₃ are, independently from each other, hydrogen, halogen or (C₁₋₄)alkyl optionally substituted by at least one halogen;
or a pharmaceutically acceptable salt thereof.

Preferred are also compounds of formula (IC) wherein
p is 1;
   or a pharmaceutically acceptable salt thereof.

Further preferred are compounds of formula (IC) having the formula

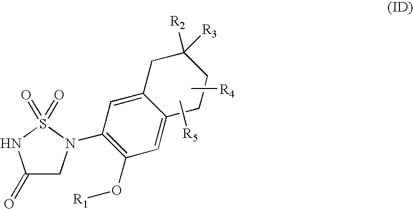

wherein
   R₁ is hydrogen or —C(O)R₆, —C(O)NR₇R₈ or —C(O)OR₉ in which
      R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
      R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
      R₂, R₃, R₄ and R₅ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
      R₂ and R₃ combined are alkylene which together with the carbon atom to which they are attached form a 3- to 7-membered spirocyclic ring;
      or a pharmaceutically acceptable salt thereof.

Preferred are compounds of formula (ID) wherein
R₄ and R₅ are hydrogen;
   or a pharmaceutically acceptable salt thereof.

Preferred are also compounds of formula (ID), designated as the B group, wherein

R₂ and R₃ are, independently from each other, hydrogen, halogen or (C₁₋₄)alkyl optionally substituted by at least one halogen;
or a pharmaceutically acceptable salt thereof.

Preferred are compounds in the B group wherein
R₁ is hydrogen or —C(O)R₆ in which R₆ is monocyclic aryl;
   or a pharmaceutically acceptable salt thereof.

Preferred are also compounds of formula (ID), designated as the C group, wherein

R₂ and R₃ combined are alkylene which together with the carbon atom to which they are attached form a 3- to 5-membered spirocyclic ring;
or a pharmaceutically acceptable salt thereof.

Preferred are compounds in the C group, wherein
R₁ is hydrogen or —C(O)R₆ in which R₆ is monocyclic aryl;
   or a pharmaceutically acceptable salt thereof.

Preferred are also compounds of formula (ID), designated as the D group, wherein
R₂ is —Y—(CH₂)ₙ—CR₁₀R₁₁—(CH₂)ₘ—X in which
   Y is oxygen or S(O)_{q} in which q is zero or an integer of 1 or 2; or
   Y is trans CH═CH; or
   Y is absent;
   n is an integer from 1 to 6;
   R₁₀ and R₁₁ are, independently from each other, hydrogen or lower alkyl; or
      R₁₀ and R₁₁ combined are alkylene which together with the carbon atom to which they are attached form a 3- to 7-membered ring;
m is zero or an integer of 1 or 2;
   X is hydroxy, alkoxy, cycloalkyl, cycloalkoxy, acyl, acyloxy, carbamoyl, cyano, trifluoromethyl, free or esterified carboxy, monocyclic aryl or heterocyclyl;
   or a pharmaceutically acceptable salt thereof.

Preferred are compounds in the D group wherein
R₃ is hydrogen;
   or a pharmaceutically acceptable salt thereof.

Further preferred are compounds in the D group wherein
n is an integer of 2 or 3;
R₁₀ and R₁₁ are, independently from each other, hydrogen or lower alkyl;
m is zero or 1;
X is hydroxy, carbamoyl, cyano, trifluoromethyl, free or esterified carboxy, monocyclic aryl or heterocyclyl;
   or a pharmaceutically acceptable salt thereof.

More preferred are compounds in the D group wherein
Y is absent;
   or a pharmaceutically acceptable salt thereof.

Even more preferred are compounds in the D group wherein
n is 3;
R₁₀ and R₁₁ are lower alkyl;
m is zero or 1;
X is hydroxy, cyano or free or esterified carboxy;
   or a pharmaceutically acceptable salt thereof.

Most preferred are compounds in the D group wherein
R₁₀ and R₁₁ are methyl;
   or a pharmaceutically acceptable salt thereof.

Especially preferred are compounds in the D group wherein
R₁ is hydrogen or —C(O)Rₑ in which R₆ is monocyclic aryl;
   or a pharmaceutically acceptable salt thereof.

Particular embodiments are:
5-(3,6-Dihydroxynaphthalen-2-yl)-1,1-dioxo-1,2,5-thiadiazolidin-3-one;
5-(3,7-Dihydroxynaphthalen-2-yl)-1,1-dioxo-1,2,5-thiadiazolidin-3-one potassium salt;
5-(7-Bromo-3-hydroxynaphthalen-2-yl)-1,1-dioxo-1,2,5-thiadiazolidin-3-one;
5-(7-Ethyl-3-hydroxynaphthalen-2-yl)-1,1-dioxo-1,2,5-thiadiazolidin-3-one;
5-{3-Hydroxy-7-[2-(4-methoxyphenyl)-ethyl]-naphthalen-2-yl}-1,1-dioxo-1,2,5-thiadiazolidin-3-one;
5-{3-Hydroxy-7-[2-(4-trifluoromethylphenyl)-ethyl]-naphthalen-2-yl}-1,1-dioxo-1,2,5-thiadiazolidin-3-one;
5-{3-Hydroxy-7-[2-(3-methoxyphenyl)-ethyl]-naphthalen-2-yl}-1,1-dioxo-1,2,5-thiadiazolidin-3-one;
5-[3-Hydroxy-7-(4-methylpentyl)-naphthalen-2-yl]-1,1-dioxo-1,2,5-thiadiazolidin-3-one;
{3-[6-Hydroxy-7-(1,1,4-trioxo-1,2,5-thiadiazolidin-2-yl)naphthalen-2-yl]-phenyl}-acetic acid;
5-(3-Hydroxy-7-phenylnaphthalen-2-yl)-1,1-dioxo-1,2,5-thiadiazolidin-3-one;
3-[6-Hydroxy-7-(1,1,4-trioxo-1,2,5-thiadiazolidin-2-yl)-naphthalen-2-yl]-benzoic acid;
5-[3-Hydroxy-7-(3-trifluoromethoxyphenyl)-naphthalen-2-yl]-1,1-dioxo-1,2,5-thiadiazolidin-3-one;
{3-[6-Hydroxy-7-(1,1,4-trioxo-1,2,5-thiadiazolidin-2-yl)-naphthalen-2-yl]-phenyl}acetonitrile;
5-[3-Hydroxy-7-(3-hydroxymethylphenyl)-naphthalen-2-yl]-1,1-dioxo-1,2,5-thiadiazolidin-3-one;
3-{3-[6-Hydroxy-7-(1,1,4-trioxo-thiadiazolidin-2-yl)-naphthalen-41]-phenyl}-propionic acid;
6-Hydroxy-7-(1,1,4-trioxo-1,2,5-thiadiazolidin-2-yl)-naphthalene-2-carbonitrile;
3-[6-Hydroxy-7-(1,1,4-trioxo-1,2,5-thiadiazolidin-2-yl)-naphthalen-2-yl]-benzonitrile;
5-[7-(3,3-Dimethylbutyl)-3-hydroxynaphthalen-2-yl]-1,1-dioxo-1,2,5-thiadiazolidin-3-one;
5-[3-Hydroxy-7-(3-trifluoromethylphenyl)-naphthalen-2-yl]-1,1-dioxo-1,2,5-thiadiazolidin-3-one;
3-[6-Hydroxy-7-(1,1,4-trioxo-1,2,5-thiadiazolidin-2-yl)-naphthalen-2-yl]-benzoic acid ethyl ester;
5-[3-Hydroxy-7-(3-methanesulfonylphenyl)-naphthalen-2-yl]-1,1-dioxo-1,2,5-thiadiazolidin-3-one;
3-{3-[6-Hydroxy-7-(1,1,4-trioxo-1,2,5-thiadiazolidin-2-yl)-naphthalen-2-yl]-phenyl}-propionitrile;
5-[3-Hydroxy-7-(3-methoxymethylphenyl)-naphthalen-2-yl]-1,1-dioxo-1,2,5-thiadiazolidin-3-one;
5-(7-Furan-3-yl-3-hydroxynaphthalen-2-yl)-1,1-dioxo-1,2,5-thiadiazolidin-3-one;
N-{3-[6-Hydroxy-7-(1,1,4-trioxo-1,2,5-thiadiazolidin-2-yl)-naphthalen-2-yl]-phenyl}-methanesulfonamide;
5-[7-(2-Fluorophenyl)-3-hydroxynaphthalen-2-yl]-1,1-dioxo-1,2,5-thiadiazolidin-3-one;
5-(3-Hydroxy-7-o-tolylnaphthalen-2-yl)-1,1-dioxo-1,2,5-thiadiazolidin-3-one;
5-(3-Hydroxy-7-pentylnaphthalen-2-yl)-1,1-dioxo-1,2,5-thiadiazolidin-3-one;
5-(3-Hydroxy-7-propylnaphthalen-2-yl)-1,1-dioxo-1,2,5-thiadiazolidin-3-one;
5-[3-Hydroxy-7-(tetrahydrofuran-3-yl)-naphthalen-2-yl]-1,1-dioxo-1,2,5-thiadiazolidin-3-one;
{3-[6-Hydroxy-7-(1,1,4-trioxo-1,2,5-thiadiazolidin-2-yl)-naphthalen-2-yl]-phenyl}-acetic acid ethyl ester;
3-{3-[6-Hydroxy-7-(1,1,4-trioxo-1,2,5-thiadiazolidin-2-yl)-naphthalen-2-yl]-phenyl}-propionic acid ethyl ester;
5-[6-Hydroxy-7-(1,1,4-trioxo-1,2,5-thiadiazolidin-2-yl)-naphthalen-2-yl]-pentanoic acid ethyl ester;
4-[6-Hydroxy-7-(1,1,4-trioxo-1,2,5-thiadiazolidin-2-yl)-naphthalen-2-yl]-2,2-dimethyl-butyric acid;
5-[3-Hydroxy-7-((S)-4-hydroxypentyl)-naphthalen-2-yl]-1,1-dioxo-1,2,5-thiadiazolidin-3-one;
4-[6-Hydroxy-7-(1,1,4-trioxo-1,2,5-thiadiazolidin-2-yl)-naphthalen-2-yl]-2-methylbutyronitrile;
5-[6-Hydroxy-7-(1,1,4-trioxo-1,2,5-thiadiazolidin-2-yl)-naphthalen-2-yl]-2-methylpentanoic acid ethyl ester;
5-[3-Hydroxy-7-(3-methylbutyl)-naphthalen-2-yl]-1,1-dioxo-1,2,5-thiadiazolidin-3-one;
5-[6-Hydroxy-7-(1,1,4-trioxo-1,2,5-thiadiazolidin-2-yl)-naphthalen-2-yl]-2,2-dimethylpentanenitrile;
5-[6-Hydroxy-7-(1,1,4-trioxo-1,2,5-thiadiazolidin-2-yl)-naphthalen-2-yl]-pentanoic acid;
5-[3-Hydroxy-7-(5-hydroxypentyl)-naphthalen-2-yl]-1,1-dioxo-1,2,5-thiadiazolidin-3-one;
2-Hydroxy-6-{2-[6-hydroxy-7-(1,1,4-trioxo-1,2,5-thiadiazolidin-2-yl)-naphthalen-2-yloxy]-ethoxy}-N,N-dimethylbenzamide;
2-Hydroxy-6-{4-[6-hydroxy-7-(1,1,4-trioxo-1,2,5-thiadiazolidin-2-yl)-naphthalen-2-yl]-butoxy}-N,N-dimethylbenzamide;
5-{3-Hydroxy-7-[3-(2-hydroxyethoxy)-propyl]-naphthalen-2-yl}-1,1-dioxo-1,2,5-thiadiazolidin-3-one;
5-{3-Hydroxy-7-[2-(2-methoxyphenyl)-ethyl]-naphthalen-2-yl}-1,1-dioxo-1,2,5-thiadiazolidin-3-one;
5-[3-Hydroxy-7-(5-oxohexyl)-naphthalen-2-yl]-1,1-dioxo-1,2,5-thiadiazolidin-3-one;
5-{7-[3-(3,5-Dimethylpyrazol-1-yl)-propyl]-3-hydroxy-naphthalen-2-yl}-1,1-dioxo-1,2,5-thiadiazolidin-3-one;
5-{3-Hydroxy-7-[3-(2-oxocyclohexyl)-propyl]-naphthalen-2-yl}-1,1-dioxo-1,2,5-thiadiazolidin-3-one;
5-{3-Hydroxy-7-[4-hydroxy-4-(tetrahydrofuran-2-yl)-butyl]-naphthalen-2-yl}-1,1-dioxo-1,2,5-thiadiazolidin-3-one;
5-{3-Hydroxy-7-[1-(2-oxopyrrolidin-1-yl)-ethyl]naphthalen-2-yl}-1,1-dioxo-1,2,5-thiadiazolidin-3-one;
5-[3-Hydroxy-7-(3-phenylpropyl)-naphthalen-2-yl]-1,1-dioxo-1,2,5-thiadiazolidin-3-one;
5-[3-Hydroxy-7-(3-pentafluorophenylpropyl)-naphthalen-2-yl]-1,1-dioxo-1,2,5-thiadiazolidin-3-one;
2-{3-[6-Hydroxy-7-(1,1,4-trioxo-1,2,5-thiadiazolidin-2-yl)-naphthalen-2-yl]-propyl}benzonitrile;
5-[3-Hydroxy-7-((R)-4-hydroxypentyl)-naphthalen-2-yl]-1,1-dioxo-1,2,5-thiadiazolidin-3-one;
5-[3-Hydroxy-7-(4-hydroxypentyl)-naphthalen-2-yl]-1,1-dioxo-1,2,5-thiadiazolidin-3-one;
5-[3-Hydroxy-7-(4-hydroxy-3-methylbutyl)-naphthalen-2-yl]-1,1-dioxo-1,2,5-thiadiazolidin-3-one;
5-[7-(4-Ethyl-4-hydroxyhexyl)-3-hydroxynaphthalen-2-yl]-1,1-dioxo-1,2,5-thiadiazolidin-3-one;
5-[3-Hydroxy-7-(4-hydroxyheptyl)-naphthalen-2-yl]-1,1-dioxo-1,2,5-thiadiazolidin-3-one;
5-{3-Hydroxy-7-[3-(1-hydroxycyclohexyl)-propyl]-naphthalen-2-yl}-1,1-1,2,5-thiadiazolidin-3-one;
5-[6-Hydroxy-7-(1,1,4-trioxo-1,2,5-thiadiazolidin-2-yl)-naphthalen-2-yl]-2,2-dimethylpentanoic acid;
5-{3-Hydroxy-7-[2-((1S,2R)-2-hydroxycyclopentyl)-ethyl]-naphthalen-2-yl}-1,1-dioxo-1,2,5-thiadiazolidin-3-one;
5-[6-Hydroxy-7-(1,1,4-trioxo-1,2,5-thiadiazolidin-2-yl)-naphthalen-2-yl]-pentanenitrile;
5-{3-Hydroxy-7-[3-(2-hydroxycyclohexyl)-propyl]-naphthalen-2-yl}-1,1-dioxo-1,2,5-thiadiazolidin-3-one;
5-[6-Hydroxy-7-(1,1,4-trioxo-1,2,5-thiadiazolidin-2-yl)-naphthalen-2-yl]-2,2-dimethylpentanoic acid methyl ester;
5-[3-Hydroxy-7-(5,5,5-trifluoro-4-hydroxy-4-methylpentyl)-naphthalen-2-yl]-1,1-dioxo-1,2,5-thiadiazolidin-3-one;
Acetic acid 4-[6-hydroxy-7-(1,1,4-trioxo-1,2,5-thiadiazolidin-2-yl)-naphthalen-2-yl]-2-methyl butyl ester;
5-[3-Hydroxy-7-(5,5,5-trifluoro-4-hydroxypentyl)-naphthalen-2-yl]-1,1-dioxo-1,2,5-thiadiazolidin-3-one;
5-[3-Hydroxy-7-(4-hydroxy-4-methylpentyl)-naphthalen-2-yl]-1,1-dioxo-1,2,5-thiadiazolidin-3-one;
5-(7-Cyclopentyl-3-hydroxynaphthalen-2-yl)-1,1-dioxo-1,2,5-thiadiazolidin-3-one;
5-(7-Cyclohexyl-3-hydroxynaphthalen-2-yl)-1,1-dioxo-1,2,5-thiadiazolidin-3-one;
5-[3-Hydroxy-7-(3-methylsulfanylphenyl)-naphthalen-2-yl]-1,1-dioxo-1,2,5-thiadiazolidin-3-one;
5-[3-Hydroxy-7-((E)-4-hydroxy-4-methylpent-1-enyl)-naphthalen-2-yl]-1,1-dioxo-1,2,5-thiadiazolidin-3-one;
5-[6-Hydroxy-7-(1,1,4-trioxo-1,2,5-thiadiazolidin-2-yl)-naphthalen-2-yl]-thiophene-2-carbonitrile;
{3-[6-Hydroxy-7-(1,1,4-trioxo-1,2,5-thiadiazolidin-2-yl)-naphthalen-2-yl]-benzyl}-carbamic acid methyl ester;
(E)-5-[6-Hydroxy-7-(1,1,4-trioxo-1,2,5-thiadiazolidin-2-yl)-naphthalen-2-yl]-pent-4-enenitrile;
(E)-5-[6-Hydroxy-7-(1,1,4-trioxo-1,2,5-thiadiazolidin-2-yl)-naphthalen-2-yl]-2-methylpent-4-enoic acid ethyl ester;
(E)-5-[6-Hydroxy-7-(1,1,4-trioxo-1,2,5-thiadiazolidin-2-yl)naphthalen-2-yl]-2-methylpent-4-enoic acid;

(E)-5-[6-Hydroxy-7-(1,1,4-trioxo-1,2,5-thiadiazolidin-2-yl)-naphthalen-2-yl]-pent-4-enoic acid;
5-[6-Hydroxy-7-(1,1,4-trioxo-1,2,5-thiadiazolidin-2-yl)-naphthalen-2-yl]-pentanoic acid isopropyl ester;
5-[6-Hydroxy-7-(1,1,4-trioxo-1,2,5-thiadiazolidin-2-yl)-naphthalen-2-yl]-2-methylpentanoic acid methyl ester;
5-[6-Hydroxy-7-(1,1,4-trioxo-1,2,5-thiadiazolidin-2-yl)-naphthalen-2-yl]-2-methylpentanoic acid;
5-[7-(4,5-Dihydroxy-4,5-dimethylhex-1-enyl)-3-hydroxynaphthalen-2-yl]-1,1-dioxo-1,2,5-thiadiazolidin-3-one;
5-[7-(4,5-Dihydroxy-4,5-dimethylhexyl)-3-hydroxynaphthalen-2-yl]-1,1-dioxo-1,2,5-thiadiazolidin-3-one;
5-[7-(4,4-Dimethylpentyl)-3-hydroxynaphthalen-2-yl]-1,1-dioxo-1,2,5-thiadiazolidin-3-one;
Benzoic acid 6-(3-cyano-3-methylpropyl)-3-(1,1,4-trioxo-1,2,5-thiadiazolidin-2-yl)-naphthalen-2-yl ester;
2,2-Dimethylpropionic acid 6-(3-cyano-3-methylpropyl)-3-(1,1,4-trioxo-1,2,5-thiadiazolidin-2-yl)-naphthalen-2-yl ester;
Propionic acid 6-(3-cyano-3-methylpropyl)-3-(1,1,4-trioxo-1,2,5-thiadiazolidin-2-yl)-naphthalen-2-yl ester;
2-Ethylbutyric acid 6-(3-cyano-3-methylpropyl)-3-(1,1,4-trioxo-1,2,5-thiadiazolidin-2-yl)-naphthalen-2-yl ester;
Hexanoic acid 6-(3-cyano-3-methylpropyl)-3-(1,1,4-trioxo-1,2,5-thiadiazolidin-2-yl)-naphthalen-2-yl ester;
2-Acetoxy-benzoic acid 6-(3-cyano-3-methylpropyl)-3-(1,1,4-trioxo-1,2,5-thiadiazolidin-2-yl)-naphthalen-2-yl ester;
Pentanoic acid 6-(3-cyano-3-methylpropyl)-3-(1,1,4-trioxo-1,2,5-thiadiazolidin-2-yl)-naphthalen-2-yl ester;
Acetic acid 6-(3-cyano-3-methylpropyl)-3-(1,1,4-trioxo-1,2,5-thiadiazolidin-2-yl)-naphthalen-2-yl ester;
3-Methylbenzoic acid 6-(3-cyano-3-methylpropyl)-3-(1,1,4-trioxo-1,2,5-thiadiazolidin-2-yl)-naphthalen-2-yl ester;
2-Methylbenzoic acid 6-(3-cyano-3-methylpropyl)-3-(1,1,4-trioxo-1,2,5-thiadiazolidin-2-yl)-naphthalen-2-yl ester;
4-Butylbenzoic acid 6-(3-cyano-3-methylpropyl)-3-(1,1,4-trioxo-1,2,5-thiadiazolidin-2-yl)-naphthalen-2-yl ester;
Cyclohexanecarboxylic acid 6-(3-cyano-3-methylpropyl)-3-(1,1,4-trioxo-1,2,5-thiadiazolidin-2-yl)-naphthalen-2-yl ester;
4-tert-Butylbenzoic acid 6-(3-cyano-3-methylpropyl)-3-(1,1,4-trioxo-1,2,5-thiadiazolidin-2-yl)-naphthalen-2-yl ester;
2,2-Dimethylpropionic acid 6-(3-cyanophenyl)-3-(1,1,4-trioxo-1,2,5-thiadiazolidin-2-yl)-naphthalen-2-yl ester;
Benzoic acid 6-(4-ethoxycarbonylbutyl)-3-(1,1,4-trioxo-1,2,5-thiadiazolidin-2-yl)-naphthalen-2-yl ester;
Benzoic acid 6-(3-methylbutyl)-3-(1,1,4-trioxo-1,2,5-thiadiazolidin-2-yl)-naphthalen-2-yl ester;
Benzoic acid 6-((E)-4-hydroxy-4-methylpent-1-enyl)-3-(1,1,4-trioxo-1,2,5-thiadiazolidin-2-yl)-naphthalen-2-yl ester;
Benzoic acid 6-methyl-3-(1,1,4-trioxo-1,2,5-thiadiazolidin-2-yl)-naphthalen-2-yl ester;
Benzoic acid 6-(5-hydroxy-4,4-dimethylpentyl)-3-(1,1,4-trioxo-1,2,5-thiadiazolidin-2-yl)-naphthalen-2-yl ester;
5-[3-Hydroxy-7-(5-hydroxy-4,4-dimethylpentyl)-naphthalen-2-yl]-1,1-dioxo-1,2,5-thiadiazolidin-3-one;
5-(3-Hydroxy-5,6,7,8-tetrahydronapthalen-2-yl)-1,1-dioxo-1,2,5-thiadiazolidin-3-one;
5-(3,6-Dihydroxy-5,6,7,8-tetrahydronaphthalen-2-yl)-1,1-dioxo-1,2,5-thiadiazolidin-3-one;
5-(3-Hydroxy-6-methoxy-5,6,7,8-tetrahydronaphthalen-2-yl)-1,1-dioxo-1,2,5-thiadiazolidin-3-one;
5-(6-Ethoxy-3-hydroxy-5,6,7,8-tetrahydronaphthalen-2-yl)-1,1-dioxo-1,2,5-thiadiazolidin-3-one;
5-(3-Hydroxy-7-methyl-5,6,7,8-tetrahydronaphthalen-2-yl)-1,1-dioxo-1,2,5-thiadiazolidin-3-one;
5-(3-Hydroxy-7,7-dimethyl-5,6,7,8-tetrahydronaphthalen-2-yl)-1,1-dioxo-1,2,5-thiadiazolidin-3-one;
5-(3-Hydroxy-7-trifluoromethyl-5,6,7,8-tetrahydronaphthalen-2-yl)-1,1-dioxo-1,2,5-thiadiazolidin-3-one;
5-(3-Hydroxy-7-isopropyl-5,6,7,8-tetrahydronaphthalen-2-yl)-1,1-dioxo-1,2,5-thiadiazolidin-3-one;
5-(7-Ethyl-3-hydroxy-5,6,7,8-tetrahydronaphthalen-2-yl)-1,1-dioxo-1,2,5-thiadiazolidin-3-one;
5-(7,7-Diethyl-3-hydroxy-5,6,7,8-tetrahydronaphthalen-2-yl)-1,1-dioxo-1,2,5-thiadiazolidin-3-one;
5-(3-Hydroxy-7,7-dipropyl-5,6,7,8-tetrahydronaphthalen-2-yl)-1,1-dioxo-1,2,5-thiadiazolidin-3-one;
5-(6′-Hydroxy-3′,4′-dihydro-1′H-spiro[cyclopentane-1,2′-naphthalen]-7′-yl)1,2,5-thiadiazolidin-3-one 1,1-dioxide;
5-((S)-7-Ethyl-3-hydroxy-5,6,7,8-tetrahydronaphthalen-2-yl)-1,1-dioxo-1,2,5-thiadiazolidin-3-one;
5-[6-Hydroxy-7-(1,1,4-trioxo-1,2,5-thiadiazolidin-2-yl)-1,2,3,4-tetrahydronaphthalen-2-yl]-2,2-dimethylpentanoic acid methyl ester;
5-[6-Hydroxy-7-(1,1,4-trioxo-1,2,5-thiadiazolidin-2-yl)-1,2,3,4-tetrahydronaphthalen-2-yl]-2,2-dimethylpentanoic acid;
5-(6-Hydroxy-2-methyl-2,3-dihydrobenzo[b]thiophen-5-yl)-1,1-dioxo-1,2,5-thiadiazolidin-3-one;
5-(6-Hydroxyindan-5-yl)-1,1-dioxo-1,2,5-thiadiazolidin-3-one;
5-(6-Hydroxy-2,2-dimethylindan-5-yl)-1,1-dioxo-1,2,5-thiadiazolidin-3-one;
5-(6-Hydroxy-2-methylindan-5-yl)-1,1-dioxo-1,2,5-thiadiazolidin-3-one;
Benzoic acid 6,6-dimethyl-3-(1,1,4-trioxo-1,2,5-thiadiazolidin-2-yl)-5,6,7,8-tetrahydronaphthalen-2-yl ester;
Benzoic acid (S)-6-ethyl-3-(1,1,4-trioxo-1,2,5-thiadiazolidin-2-yl)-5,6,7,8-tetrahydronaphthalen-2-yl ester;
Benzoic acid 6-ethyl-3-(1,1,4-trioxo-1,2,5-thiadiazolidin-2-yl)-5,6,7,8-tetrahydronaphthalen-2-yl ester;
Benzoic acid 6,6-diethyl-3-(1,1,4-trioxo-1,2,5-thiadiazolidin-2-yl)-5,6,7,8-tetrahydronaphthalen-2-yl ester;
Benzoic acid 2,2-dimethyl-6-(1,1,4-trioxo-1,2,5-thiadiazolidin-2-yl)-indan-5-yl ester;
5-(3-Allyloxy-6-hydroxybenzo[c]isoxazol-5-yl)-1,1-dioxo-1,2,5-thiadiazolidin-3-one;
5-Hydroxy-6-(1,1,4-trioxo-1,2,5-thiadiazolidin-2-yl)-1H-indole-2-carboxylic acid ethyl ester potassium salt;
5-Hydroxy-6-(1,1,4-trioxo-1,2,5-thiadiazolidin-2-yl)-1H-indole-2-carboxylic acid 3-methyl-butyl ester;
5-Hydroxy-6-(1,1,4-trioxo-1,2,5-thiadiazolidin-2-yl)-1H-indole-2-carboxylic acid isobutyl ester;
5-Hydroxy-6-(1,1,4-trioxo-1,2,5-thiadiazolidin-2-yl)-1H-indole-2-carboxylic acid; and
5-(7-Hydroxy-3-methoxy-2-oxo-2H-chromen-6-yl)-1,1-dioxo-1,2,5-thiadiazolidin-3-one;
5-(3-Hydroxy-7-methoxynaphthalen-2-yl)-1,1-dioxo-[1,2,5]thiadiazolidin-3-one;
5-(3-Hydroxy-7-methoxynaphthalen-2-yl)-1,1-dioxo-[1,2,5]thiadiazolidin-3-one potassium salt;
5-(3-Hydroxy-7-propoxynaphthalen-2-yl)-1,1-dioxo-[1,2,5]thiadiazolidin-3-one;
5-(3-Hydroxy-7-propoxynaphthalen-2-yl)-1,1-dioxo-[1,2,5]thiadiazolidin-3-one potassium salt;
5-(3-Hydroxynaphthalen-2-yl)-1,1-dioxo-[1,2,5]thiadiazolidin-3-one;
5-(3-Hydroxynaphthalen-2-yl)-1,1-dioxo-[1,2,5]thiadiazolidin-3-one potassium salt;
5-(3-Hydroxy-7-methyl-naphthalen-2-yl)-1,1-dioxo-1,2,5-thiadiazolidin-3-one; and
5-(3-Hydroxy-7-methyl-naphthalen-2-yl)-1,1-dioxo-1,2,5-thiadiazolidin-3-one potassium salt
   or a pharmaceutically acceptable salt thereof.

### Category 2 PTP Inhibitors

The methods of the invention can be practiced with the compounds of the formula

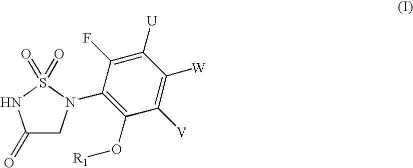

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   U, W and V are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, aryloxy, arylthio, heterocyclyl, heterocycloyloxy, alkenyl, alkynyl or (C₁₋₅)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   U and W combined together with the carbon atoms to which they are attached form an optionally substituted aromatic, or a partially or fully saturated nonaromatic 5- to 8-membered carbocyclic or heterocyclic ring; or
   W and V combined together with the carbon atoms to which they are attached form an optionally substituted aromatic, or partially or fully saturated nonaromatic 5- to 8-membered carbocyclic or heterocyclic ring;
   or a pharmaceutically acceptable salt thereof.

Preferred are the compounds of formula (I) wherein

U and W combined together with the carbon atoms to which they are attached form an optionally substituted aromatic, or a partially or fully saturated nonaromatic 5- to 8-membered carbocyclic or heterocyclic ring;
V is hydrogen;
or a pharmaceutically acceptable salt thereof.

Further preferred are the compounds of formula (I) having the formula

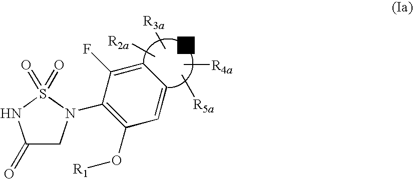

wherein
   Qₐ combined together with the carbon atoms to which it is attached form an aromatic, or a partially or fully saturated nonaromatic 5- to 8-membered carbocyclic or heterocyclic ring;
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ₐ, R₃ₐ, R₄ₐ and R₅ₐ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ₐ and R₃ₐ combined are alkylene which together with the ring atoms to which they are attached form a 3- to 7-membered fused ring; or
   R₂ₐ and R₃ₐ combined are alkylene which together with the carbon atom to which they are attached form a 3- to 7-membered spirocyclic ring;
   or a pharmaceutically acceptable salt thereof.

Preferred are compounds of formula (Ia), designated as the A group, wherein

Qₐ combined together with the carbon atoms to which it is attached form an aromatic, or a partially or fully saturated 5- to 6-membered carbocyclic ring;
or a pharmaceutically acceptable salt thereof.

Preferred are compounds in the A group having the formula

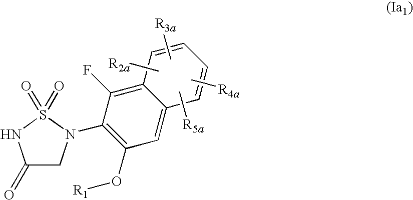

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ₐ, R₃ₐ, R₄ₐ and R₅ₐ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₅)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ₐ and R₃ₐ combined are alkylene which together with the ring atoms to which they are attached form a 5- to 7-membered fused ring; or
   or a pharmaceutically acceptable salt thereof.

Preferred are compounds of formula (Ia₁) wherein
R₄ₐ and R₅ₐ are hydrogen;
   or a pharmaceutically acceptable salt thereof.

Further preferred are compounds of formula (Ia₁) having the formula

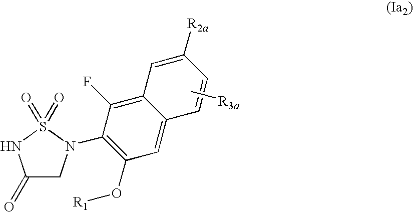

wherein
   R₁ is hydrogen, —C(O)R₂, —C(O)NR₃R₄ or —C(O)OR₅ in which
      R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
      R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
      R₂ₐ and R₃ₐ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
      or a pharmaceutically acceptable salt thereof.

Preferred are compounds of formula (Ia₂) wherein
R₂ₐ is —Yₐ—(CH₂)ₙ—CR₆ₐR₇ₐ—(CH₂)ₘ—Xₐ in which
   Yₐ is oxygen or S(O)_{q} in which q is zero or an integer of 1 or 2; or
   Yₐ is trans CH═CH; or
   Yₐ is absent;
   n is an integer from 1 to 6;
   R₆ₐ and R₇ₐ are, independently from each other, hydrogen or lower alkyl; or
      R₆ₐ and R₇ₐ combined are alkylene which together with the carbon atom to which they are attached form a 3- to 7-membered ring;
m is zero or an integer of 1 or 2;
   Xₐ is hydroxy, alkoxy, cycloalkyl, cycloalkoxy, acyl, acyloxy, carbamoyl, cyano, trifluoromethyl, free or esterified carboxy, monocyclic aryl or heterocyclyl;
   or a pharmaceutically acceptable salt thereof.

Further preferred are compounds of formula (Ia₂) wherein
R₃ₐ is hydrogen;
   or a pharmaceutically acceptable salt thereof.

Further preferred are also compounds of formula (Ia₂) wherein
n is an integer of 2 or 3;
R₆ₐ and R₇ₐ are, independently from each other, hydrogen or lower alkyl;
m is zero or 1;
   Xₐ is hydroxy, carbamoyl, cyano, trifluoromethyl, free or esterified carboxy, monocyclic aryl or heterocyclyl;
   or a pharmaceutically acceptable salt thereof.

More preferred are compounds of formula (Ia₂) wherein
Yₐ is absent;
   or a pharmaceutically acceptable salt thereof.

Even more preferred are compounds of formula (Ia₂) wherein
n is 3;
R₆ₐ and R₇ₐ are lower alkyl;
m is zero or 1;
Xₐ is hydroxy, cyano or free or esterified carboxy;
   or a pharmaceutically acceptable salt thereof.

Most preferred are compounds of formula (Ia₂) wherein
R₆ₐ and R₇ₐ are methyl;
   or a pharmaceutically acceptable salt thereof.

Especially preferred are compounds of formula (Ia₂) wherein
R₁ is hydrogen or —C(O)R₂ in which R₂ is monocyclic aryl;
   or a pharmaceutically acceptable salt thereof.

Preferred are also compounds in the A group having the formula

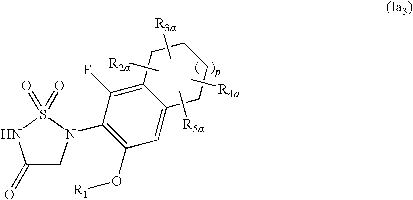

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ₐ, R₃ₐ, R₄ₐ and R₅ₐ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ₐ and R₃ₐ combined are alkylene which together with the ring atoms to which they are attached form a 3- to 7-membered fused ring; or
   R₂ₐ and R₃ₐ combined are alkylene which together with the carbon atom to which they are attached form a 3- to 7-membered spirocyclic ring;
   p is zero or 1;
   or a pharmaceutically acceptable salt thereof.

Preferred are compounds of formula (Ia₃) wherein
R₄ₐ and R₅ₐ are hydrogen;
   or a pharmaceutically acceptable salt thereof.

Preferred are also compounds of formula (Ia₃) wherein

R₂ₐ and R₃ₐ are, independently from each other, hydrogen, halogen or (C₁₋₄)alkyl optionally substituted by at least one halogen;
or a pharmaceutically acceptable salt thereof.

Preferred are also compounds of formula (Ia₃) wherein
p is 1;
   or a pharmaceutically acceptable salt thereof.

Further preferred are compounds of formula (Ia₃) having the formula

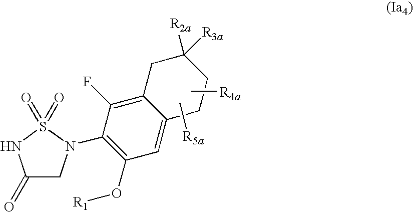

wherein
   R₁ is hydrogen, —C(O)R₂, —C(O)NR₃R₄ or —C(O)OR₅ in which
      R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
      R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
      R₂ₐ, R₃ₐ, R₄ₐ and R₅ₐ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
      R₂ₐ and R₃ₐ combined are alkylene which together with the carbon atom to which they are attached form a 3- to 7-membered spirocyclic ring;
      or a pharmaceutically acceptable salt thereof.

Preferred are compounds of formula (Ia₄) wherein
R₄ₐ and R₅ₐ are hydrogen;
   or a pharmaceutically acceptable salt thereof.

Preferred are also compounds of formula (Ia₄), designated as the B group, wherein

R₂ₐ and R₃ₐ are, independently from each other, hydrogen, halogen or (C₁₋₄)alkyl optionally substituted by at least one halogen;
or a pharmaceutically acceptable salt thereof.

Preferred are compounds in the B group wherein
R₁ is hydrogen or —C(O)R₂ in which R₂ is monocyclic aryl;
   or a pharmaceutically acceptable salt thereof.

Preferred are also compounds of formula (Ia₄), designated as the C group, wherein

R₂ₐ and R₃ₐ combined are alkylene which together with the carbon atom to which they are attached form a 3- to 5-membered spirocyclic ring;
or a pharmaceutically acceptable salt thereof.

Preferred are compounds in the C group, wherein
R₁ is hydrogen or —C(O)R₂ in which R₂ is monocyclic aryl;

or a pharmaceutically acceptable salt thereof.

Preferred are also compounds of formula (Ia₄), designated as the D group, wherein
R₂ₐ is —Yₐ—(CH₂)ₐ—CR₆ₐR₇ₐ(CH₂)ₘ—Xₐ in which
   Yₐ is oxygen or S(O)_{q} in which q is zero or an integer of 1 or 2; or
   Yₐ is trans CH═CH; or
   Yₐ is absent;
   n is an integer from 1 to 6;
   R₆ₐ and R₇ₐ are, independently from each other, hydrogen or lower alkyl; or
      R₆ₐ and R₇ₐ combined are alkylene which together with the carbon atom to which they are attached form a 3- to 7-membered ring;
m is zero or an integer of 1 or 2;
   Xₐ is hydroxy, alkoxy, cycloalkyl, cycloalkoxy, acyl, acyloxy, carbamoyl, cyano, trifluoromethyl, free or esterified carboxy, monocyclic aryl or heterocyclyl;
   or a pharmaceutically acceptable salt thereof.

Preferred are compounds in the D group wherein
R₃ₐ is hydrogen;
   or a pharmaceutically acceptable salt thereof.

Further preferred are compounds in the D group wherein
n is an integer of 2 or 3;
R₆ₐ and R₇ₐ are, independently from each other, hydrogen or lower alkyl;
m is zero or 1;
Xₐ is hydroxy, carbamoyl, cyano, trifluoromethyl, free or esterified carboxy, monocyclic aryl or heterocyclyl;
   or a pharmaceutically acceptable salt thereof.

More preferred are compounds in the D group wherein
Yₐ is absent;
   or a pharmaceutically acceptable salt thereof.

Even more preferred are compounds in the D group wherein
n is 3;
R₆ₐ and R₇ₐ are lower alkyl;
m is zero or 1;
Xₐ is hydroxy, cyano or free or esterified carboxy;
   or a pharmaceutically acceptable salt thereof.

Most preferred are compounds in the D group wherein
R₆ₐ and R₇ₐ are methyl;
   or a pharmaceutically acceptable salt thereof.

Especially preferred are compounds in the D group wherein
R₁ is hydrogen or —C(O)R₂ in which R₂ is monocyclic aryl;
   or a pharmaceutically acceptable salt thereof.

Preferred are also the compounds of formula (I), wherein
U and V are hydrogen;
W is aryloxy, arylthio or methyl substituted with monocyclic aryl;
   or a pharmaceutically acceptable salt thereof.

Further preferred are also the compounds of formula (I) having the formula

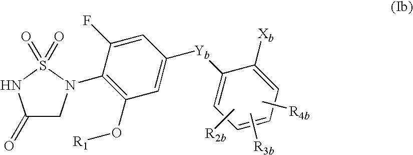

wherein
   R₁ is hydrogen, —C(O)R₂, —C(O)NR₃R₄ or —C(O)OR₅ in which
      R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
      R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
      R_{2b}, R_{3b} and R_{4b} are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₅)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
      R_{2b} and R_{3b} combined are alkylene which together with the ring atoms to which they are attached form a 5- to 7-membered fused ring provided R₂ and R₃ are attached to carbon atoms adjacent to each other; or
      R_{2b} and R_{3b} combined together with the carbon atom to which they are attached form a fused 5- to 6-membered aromatic or heteroaromatic ring provided R₂ and R₃ are attached to carbon atoms adjacent to each other;
      X_{b} is hydrogen, fluoro, cyano, or free or esterified carboxy; or
      X_{b} is —NR_{5b}C(O)R_{6b}, —NR_{5b}C(O)OR_{7b}, —NR_{5b}S(O)₂R_{8b}, —(CH₂)ᵣS(O)₂R_{9b}, —OS(O)₂R_{10b} or —OₛC(O)NR_{11b}R_{12b} in which
R_{5b} is hydrogen, lower alkyl, acyl, alkoxycarbonyl or sulfonyl;
   R_{6b}, R_{7b}, R_{8b}, R_{9b} and R_{10b} are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R_{6b}, R_{8b} and R_{9b} are, independently from each other, —NR_{13b}R_{14b} in which R_{13b} and R_{14b} are, independently from each other, hydrogen, alkyl, cycloalkyl, aralkyl, aryl or heterocyclyl; or
   R_{13b} and R_{14b} combined are alkylene which together with the nitrogen atom to which they are attached form a 4- to 7-membered ring;
   R_{11b} and R_{12b} are, independently from each other, hydrogen, alkyl, cycloalkyl, aralkyl, aryl or heterocyclyl; or
   R_{11b} and R_{12b} combined are alkylene which together with the nitrogen atom to which they are attached form a 4- to 7-membered ring;
   r and s are, independently from each other, zero or an integer of 1; or
   C—X_{b} is replaced by nitrogen;

### Y_{b} is O, S or CH₂;

or a pharmaceutically acceptable salt thereof.

Preferred are the compounds of formula (Ib) wherein

### Y_{b} is CH₂,

or a pharmaceutically acceptable salt thereof.

Further preferred are the compounds of formula (Ib) having the formula

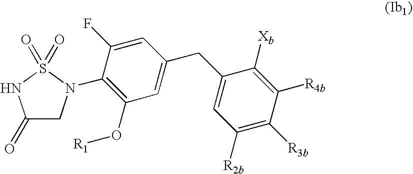

wherein
R₁ is hydrogen, —C(O)R₂, —C(O)NR₃R₄ or —C(O)OR₅ in which
R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R_{2b}, R_{3b} and R_{4b} are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
R_{2b} and R_{3b} combined are alkylene which together with the ring atoms to which they are attached form a 5- to 7-membered fused ring; or
R_{2b} and R_{3b} combined together with the carbon atom to which they are attached form a fused 5- to 6-membered aromatic or heteroaromatic ring;
X_{b} is cyano; or
X_{b} is —NR_{5b}C(O)R_{6b}, —NR_{5b}C(O)OR_{7b}, —NR_{5b}S(O)₂R_{8b}, —(CH₂)ᵣS(O)₂R_{9b} or —OS(O)₂R_{10b} in which
R_{5b} is hydrogen or lower alkyl;
   R_{6b}, R_{7b}, R_{8b}, R₁₃ and R_{10b} are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R_{6b}, R_{8b} and R_{9b} are, independently from each other, —NR_{13b}R_{14b} in which
   R_{13b} and R_{14b} are, independently from each other, hydrogen, alkyl, cycloalkyl, aralkyl, aryl or heterocyclyl; or
   R_{13b} and R_{14b} combined are alkylene which together with the nitrogen atom to which they are attached form a 4- to 7-membered ring;
   r is zero; or
   C—X_{b} is replaced by nitrogen;
   or a pharmaceutically acceptable salt thereof.

Preferred are the compounds of formula (Ib₁) wherein

X_{b} is cyano; or
X_{b} is —NR_{5b}S(O)₂R_{9b} or —OS(O)₂R_{10b} in which
R_{5b} is hydrogen or lower alkyl;
R_{8b} and R_{10b} are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
or a pharmaceutically acceptable salt thereof.

Especially preferred are the compounds of formula (Ib₁), designated as the E group, wherein

R_{5b} is hydrogen;
or a pharmaceutically acceptable salt thereof.

Preferred are the compounds in the E group wherein

R_{8b} and R_{10b} are, independently from each other, monocyclic aryl or C₍₁₋₄)alkyl;
or a pharmaceutically acceptable salt thereof.

Further preferred are the compounds in the E group wherein

R₁ is hydrogen or —C(O)R₂ in which R₂ is monocyclic aryl;
or a pharmaceutically acceptable salt thereof.

Especially preferred are also the compounds of formula (Ib₁), designated as the F group, wherein

R_{2b}, R_{3b} and R_{4b} are, independently from each other, hydrogen, halogen, hydroxy, monocyclic aryl, C₍₁₋₄₎alkoxy or C₍₁₋₄₎alkyl optionally substituted with at least one halogen;
or a pharmaceutically acceptable salt thereof.

Preferred are the compounds in the F group wherein

R_{5b} is hydrogen;
or a pharmaceutically acceptable salt thereof.

Further preferred are the compounds in the F group wherein

R_{8b} and R_{10b} are, independently from each other, monocyclic aryl or C₍₁₋₄₎alkyl;
or a pharmaceutically acceptable salt thereof.

More preferred are the compounds in the F group wherein

R₁ is hydrogen or —C(O)R₂ in which R₂ is monocyclic aryl;
or a pharmaceutically acceptable salt thereof.

Preferred are also the compound of formula (I), designated as the G group, wherein

R₁ is hydrogen, —C(O)R₂, —C(O)NR₃R₄ or —C(O)OR₅ in which
R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
U is alkoxy, alkylthio, alkylthiono, sulfonyl, cycloalkyl, aryl, aryloxy, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
W and V are, independently from each other, hydrogen, halogen, (C₁₋₃)alkyl or (C₁₋₃)alkoxy;
or a pharmaceutically acceptable salt thereof.

Preferred are the compounds in the G group, designated as the H group, wherein

U is —Y_{c}—(CH₂)ₚ—CR_{2c}R_{3c}—(CH₂)ₜ—X_{c} in which
Y_{c} is oxygen or S(O)ᵥ in which v is zero or an integer of 1 or 2; or

### Y_{c} is C≡C; or

Y_{c} is absent;
p and t are, independently from each other, zero or an integer from 1 to 8;
R₂ and R₃, are, independently from each other, hydrogen or lower alkyl; or
R₂ and R₃, combined are alkylene which together with the carbon atom to which they are attached form a 3- to 7-membered ring;
X_{c} is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
or a pharmaceutically acceptable salt thereof.

Preferred are the compounds in the H group wherein

R₂, and R_{ic} are hydrogen;
or a pharmaceutically acceptable salt thereof.

Further preferred are the compounds in the H group wherein

p is zero or an integer from 1 to 3;
t is zero or 1;
R₂ and R₃ are, independently from each other, hydrogen or lower alkyl;
X_{c} is hydroxy, carbamoyl, cyano, trifluoromethyl, free or esterified carboxy, heterocyclyl, monocyclic aryl or monocyclic aryloxy;
or a pharmaceutically acceptable salt thereof.

Especially preferred are the compounds in the H group, designated as the I group, wherein

### Y_{c} is C≡C; or

Y_{c} is absent;
or a pharmaceutically acceptable salt thereof.

Preferred are the compounds in the I group wherein

Y_{c} is absent;
p is an integer of 5 or 6;
t is zero or 1;
R₂ and R₃ are lower alkyl;
X_{c} is hydroxy, cyano or free or esterified carboxy;
or a pharmaceutically acceptable salt thereof.

Further preferred are the compounds in the I group wherein

R_{2c} and R_{3c} are methyl;
or a pharmaceutically acceptable salt thereof.

Especially preferred are the compounds in the I group wherein

R₁ is hydrogen or —C(O)R₂ in which R₂ is monocyclic aryl;
or a pharmaceutically acceptable salt thereof.

Preferred are also the compounds in the I group, designated as the J group, wherein

Y_{c} is absent;
p is an integer of 4 or 5;
t is zero;
R_{2c} and R_{3C} are hydrogen;
X_{c} is monocyclic aryloxy;
or a pharmaceutically acceptable salt thereof.

Preferred are the compounds in the J group wherein

R₁ is hydrogen or —C(O)R₂ in which R₂ is monocyclic aryl;
or a pharmaceutically acceptable salt thereof.

Preferred are also the compounds in the J group, designated as the K group, wherein

### Y_{c} is CE C;

p is an integer of 2 or 3;
t is zero;
R_{2c} and R_{3c} are hydrogen;
X_{c} is hydroxy, cyano or free or esterified carboxy;
or a pharmaceutically acceptable salt thereof.

Preferred are the compounds in the K group wherein

R₁ is hydrogen or —C(O)R₂ in which R₂ is monocyclic aryl;
or a pharmaceutically acceptable salt thereof.

Preferred are also the compounds in the G group, designated as the L group, wherein

Q_{c} is monocyclic aryl or 5- to 6-membered heterocyclic ring;
or a pharmaceutically acceptable salt thereof.

Preferred are the compounds in the L group, designated as the M group, wherein

R_{2c} and R_{3c} are hydrogen;
or a pharmaceutically acceptable salt thereof.

Preferred are the compounds in the M group having the formula

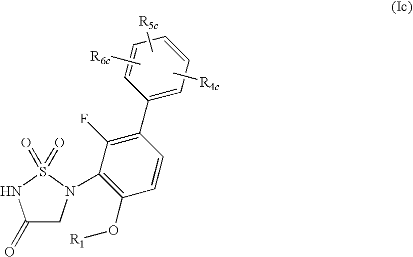

wherein
R₁ is hydrogen, —C(O)R₂, —C(O)NR₃R₄ or —C(O)OR₅ in which
R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R_{4c}, R_{5c} and R_{6c} are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
C—R_{4c}, C—R_{5c} and C—R_{6c} are, independently from each other, replaced by nitrogen;
or a pharmaceutically acceptable salt thereof.

Preferred are the compounds of formula (Ic) wherein

R_{4c} and R_{5c} are hydrogen;
or a pharmaceutically acceptable salt thereof.

Preferred are also the compounds of formula (Ic) wherein

R₁ is hydrogen or —C(O)R₂ in which R₂ is monocyclic aryl;
or a pharmaceutically acceptable salt thereof.

Preferred are also the compounds in the M group having the formula

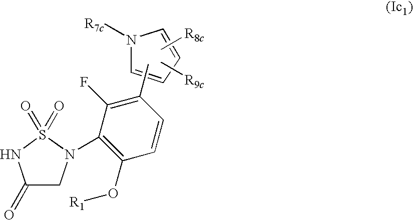

wherein
R₁ is hydrogen, —C(O)R₂, —C(O)NR₃R₄ or —C(O)OR₅ in which
R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R_{7c} is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
R_{5c} and R_{9c} are, independently from each other, hydrogen or lower alkyl; or
C—R_{5c} and C—R_{9c} are, independently from each other, replaced by nitrogen;
or a pharmaceutically acceptable salt thereof.

Preferred are the compounds of formula (IC₁) wherein

C—R_{8c} is replaced by nitrogen;
R_{9c} is hydrogen;
or a pharmaceutically acceptable salt thereof.

Further preferred are the compounds of formula (Ic₁) having the formula

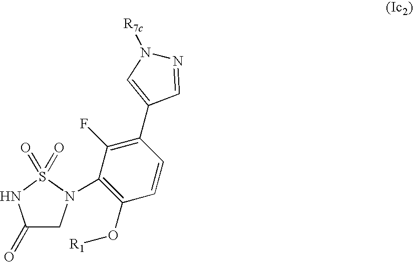

wherein
R₁ is hydrogen, —C(O)R₂, —C(O)NR₃R₄ or —C(O)OR₅ in which
R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R_{7c} is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
or a pharmaceutically acceptable salt thereof.

Preferred are the compounds of formula (Ic₂) wherein

R_{7c} is —(CH₂)ₚ—CR_{10c}R_{11c}—(CH₂)ᵣZ_{c} in which
p and t are, independently from each other, zero or an integer from 1 to 6;
R_{10c} and R_{11c} are, independently from each other, hydrogen or lower alkyl; or
R_{10c} and R_{11c} combined are alkylene which together with the carbon atom to which they are attached form a 3- to 7-membered ring;
Z_{c} is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
or a pharmaceutically acceptable salt thereof.

Further preferred are the compounds of formula (Ic₂) wherein

p is an integer from 1 to 3;
t is zero or 1;
R_{10c} and R_{11c} are, independently from each other, hydrogen or lower alkyl;
Z_{c} is hydroxy, carbamoyl, cyano, trifluoromethyl, free or esterified carboxy, heterocyclyl, monocyclic aryl or monocyclic aryloxy;
or a pharmaceutically acceptable salt thereof.

More preferred are the compounds of formula (Ic₂) wherein

R_{10c} and R_{11c} are hydrogen;
Z_{c} is hydroxy, cyano or free or esterified carboxy;
or a pharmaceutically acceptable salt thereof.

Most preferred are the compounds of formula (Ic₂) wherein

R₁ is hydrogen or —C(O)R₂ in which R₂ is monocyclic aryl;
or a pharmaceutically acceptable salt thereof.

Particular embodiments of the compounds are:
Methanesulfonic acid 2-[3-fluoro-5-hydroxy-4-(1,1,4-trioxo-1,2,5-thiadiazolidin-2-yl)-benzyl]-4-methylphenyl ester;
Methanesulfonic acid 2-[3-fluoro-5-hydroxy-4-(1,1,4-trioxo-1,2,5-thiadiazolidin-2-yl)-benzyl]-5-methylphenyl ester;
Methanesulfonic acid 2-[3-fluoro-5-hydroxy-4-(1,1,4-trioxo-1,2,5-thiadiazolidin-2-yl)-benzyl]-6-methylphenyl ester;
Methanesulfonic acid 2-[3-fluoro-5-hydroxy-4-(1,1,4-trioxo-1,2,5-thiadiazolidin-2-yl)-benzyl]-phenyl ester;
N-{2-[3-Fluoro-5-hydroxy-4-(1,1,4-trioxo-1,2,5-thiadiazolidin-2-yl)-benzyl]-5-methylphenyl}-methanesulfonamide;
N-{2-[3-Fluoro-5-hydroxy-4-(1,1,4-trioxo-1,2,5-thiadiazolidin-2-yl)-benzyl]-4-methylphenyl}-methanesulfonamide;
N-{2-[3-Fluoro-5-hydroxy-4-(1,1,4-trioxo-1,2,5-thiadiazolidin-2-yl)-benzyl]-phenyl}-methanesulfonamide;
5-(4-Benzyl-2-fluoro-6-hydroxy-phenyl)-1,1-dioxo-1,2,5-thiadiazolidin-3-one;
5-(2-Fluoro-6-hydroxy-4-methylphenyl)-1,1-dioxo-1,2,5-thiadiazolidin-3-one;
Benzoic acid 5-benzyl-3-fluoro-2-(1,1,4-trioxo-1,2,5-thiadiazolidin-2-yl)-phenyl ester;
Benzoic acid 3-fluoro-5-methyl-2-(1,1,4-trioxo-1,2,5-thiadiazolidin-2-yl)-phenyl ester;
5-(4-Cyclobutylmethyl-2-fluoro-6-hydroxyphenyl)-1,1-dioxo-1,2,5-thiadiazolidin-3-one potassium salt;
5-(4-Cyclohexylmethyl-2-fluoro-6-hydroxyphenyl)-1,1-dioxo-1,2,5-thiadiazolidin-3-one;
7-[2-Fluoro-4-hydroxy-3-(1,1,4-trioxo-1,2,5-thiadiazolidin-2-yl)-phenyl]-2,2-dimethylheptanenitrile;
5-(2,4-Difluoro-6-hydroxyphenyl)-1,1-dioxo-1,2,5-thiadiazolidin-3-one;
5-(1-Fluoro-3-hydroxy-7-methylnaphthalen-2-yl)-1,1-dioxo-1,2,5-thiadiazolidin-3-one;
5-(1-Fluoro-3-hydroxynaphthalen-2-yl)-1,1-dioxo-1,2,5-thiadiazolidin-3-one;
5-(7-Ethyl-1-fluoro-3-hydroxy-5,6,7,8-tetrahydronaphthalen-2-yl)-1,1-dioxo-1,2,5-thiadiazolidin-3-one;
5-[1-Fluoro-3-hydroxy-7-(5-hydroxy-4,4-dimethylpentyl)-naphthalen-2-yl]-1,1-dioxo-1,2,5-thiadiazolidin-3-one;
5-[8-Fluoro-6-hydroxy-7-(1,1,4-trioxo-1,2,5-thiadiazolidin-2-yl)-naphthalen-2-yl]-2,2-dimethyl-pentanoic acid;
Benzoic acid 4-fluoro-6-methyl-3-(1,1,4-trioxo-1,2,5-thiadiazolidin-2-yl)-naphthalen-2-yl ester;
Benzoic acid 6-ethyl-4-fluoro-3-(1,1,4-trioxo-1,2,5-thiadiazolidin-2-yl)-5,6,7,8-tetrahydronaphthalen-2-yl ester;
Benzoic acid 4-fluoro-3-(1,1,4-trioxo-1,2,5-thiadiazolidin-2-yl)-naphthalen-2-yl ester;
Benzoic acid 4-fluoro-6-(5-hydroxy-4,4-dimethylpentyl)-3-(1,1,4-trioxo-1,2,5-thiadiazolidin-2-yl)-naphthalen-2-yl ester;
Benzoic acid 3-fluoro-5-(2-methanesulfonyloxy-5-methylbenzyl)-2-(1,1,4-trioxo-1,2,5-thiadiazolidin-2-yl)-phenyl ester;
Benzoic acid 3-fluoro-5-(2-methanesulfonyloxy-4-methylbenzyl)-2-(1,1,4-trioxo-1,2,5-thiadiazolidin-2-yl)-phenyl ester;
Benzoic acid 4-(6-cyano-6,6-dimethylhexyl)-3-fluoro-2-(1,1,4-trioxo-1,2,5-thiadiazolidin-2-yl)-phenyl ester;
Benzoic acid 3-fluoro-5-(2-methanesulfonylamino-5-methylbenzyl)-2-(1,1,4-trioxo-1,2,5-thiadiazolidin-2-yl)-phenyl ester;
Benzoic acid 3-fluoro-5-(2-methanesulfonylamino-4-methylbenzyl)-2-(1,1,4-trioxo-1,2,5-thiadiazolidin-2-yl)-phenyl ester; and
Benzoic acid 3-fluoro-5-(2-methanesulfonyloxy-3-methylbenzyl)-2-(1,1,4-trioxo-1,2,5-thiadiazolidin-2-yl)-phenyl ester;
   or a pharmaceutically acceptable salt thereof.

### Category 3 PTP Inhibitors

The methods of the present invention can be practiced with the compounds of the formula

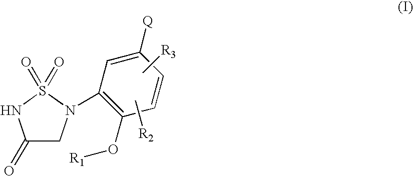

wherein
Q is alkoxy, alkylthio, alkylthiono, sulfonyl, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
R₁ is hydrogen, —C(O)R₄, —C(O)NR₅R₆ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂ and R₃ are, independently from each other, hydrogen, halogen, (C₁₋₃)alkyl or (C₁₋₃)alkoxy;
or a pharmaceutically acceptable salt thereof.

Preferred are the compounds of formula (I), designated as the A group, wherein

Q is —Y—(CH₂)ₙ—CR₈R₉—(CH₂)ₘ—X in which
Y is oxygen or S(O)_{q} in which q is zero or an integer of 1 or 2; or

### Y is C≡C; or

Y is absent;
n and m are, independently from each other, zero or an integer from 1 to 8;
R₈ and R₉ are, independently from each other, hydrogen or lower alkyl; or
R₈ and R₉ combined are alkylene which together with the carbon atom to which they are attached form a 3- to 7-membered ring;
X is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
or a pharmaceutically acceptable salt thereof.

Preferred are the compounds in the A group wherein

R₂ and R₃ are hydrogen;
or a pharmaceutically acceptable salt thereof.

Further preferred are the compounds in the A group wherein

n is zero or an integer from 1 to 3;
m is zero or 1;
R₈ and R₉ are, independently from each other, hydrogen or lower alkyl;
X is hydroxy, carbamoyl, cyano, trifluoromethyl, free or esterified carboxy, heterocyclyl, monocyclic aryl or monocyclic aryloxy;
or a pharmaceutically acceptable salt thereof.

Especially preferred are the compounds in the A group, designated as the B group, wherein

### Y is C≡C; or

Y is absent;
or a pharmaceutically acceptable salt thereof.

Preferred are the compounds in the B group wherein

Y is absent;
n is an integer of 5 or 6;
m is zero or 1;
R₈ and R₉ are lower alkyl;
X is hydroxy, cyano or free or esterified carboxy;
or a pharmaceutically acceptable salt thereof.

Further preferred are the compounds in the B group wherein

R₈ and R₉ are methyl;
or a pharmaceutically acceptable salt thereof.

Especially preferred are the compounds in the B group wherein

R₁ is hydrogen or —C(O)R₄ in which R₄ is monocyclic aryl;
or a pharmaceutically acceptable salt thereof.

Preferred are also the compounds in the B group, designated as the C group, wherein

Y is absent;
n is an integer of 4 or 5;
m is zero;
R₈ and R₉ are hydrogen;
X is monocyclic aryloxy;
or a pharmaceutically acceptable salt thereof.

Preferred are the compounds in the C group wherein

R₁ is hydrogen or —C(O)R₄ in which R₄ is monocyclic aryl;
or a pharmaceutically acceptable salt thereof.

Preferred are also the compounds in the B group, designated as the D group, wherein

### Y is C≡C;

n is an integer of 2 or 3;
m is zero;
R₈ and R₉ are hydrogen;
X is hydroxy, cyano or free or esterified carboxy;
or a pharmaceutically acceptable salt thereof.

Preferred are the compounds in the D group wherein

R₁ is hydrogen or —C(O)R₄ in which R₄ is monocyclic aryl;
or a pharmaceutically acceptable salt thereof.

Preferred are the compounds of formula (I), designated as the E group, wherein

Q is monocyclic aryl or 5- to 6-membered heterocyclic ring;
or a pharmaceutically acceptable salt thereof.

Preferred are the compounds in the E group, designated as the G group, wherein

R₂ and R₃ are hydrogen;
or a pharmaceutically acceptable salt thereof.

Preferred are the compounds in the G group having the formula

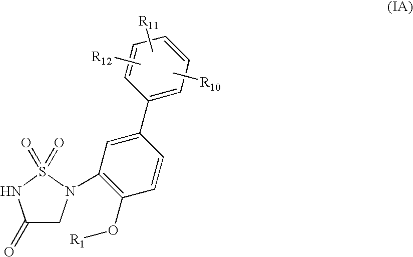

wherein
R₁ is hydrogen, —C(O)R₄, —C(O)NR₅R₆ or —C(O)OR₇ in which R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₁₀, R₁₁ and R₁₂ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
C—R₁₀, C—R₁₁ and C—R₁₂ are, independently from each other, replaced by nitrogen;
or a pharmaceutically acceptable salt thereof.

Preferred are the compounds of formula (IA) wherein

R₁₀ and R₁₁ are hydrogen;
or a pharmaceutically acceptable salt thereof.

Preferred are also the compounds of formula (IA) wherein

R₁ is hydrogen or —C(O)R₄ in which R₄ is monocyclic aryl;
or a pharmaceutically acceptable salt thereof.

Preferred are also the compounds in the G group having the formula

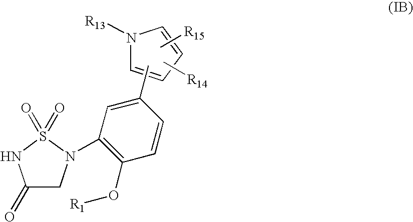

wherein
R₁ is hydrogen, —C(O)R₄, —C(O)NR₅R₆ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₁₃ is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
R₁₄ and R₁₅ are, independently from each other, hydrogen or lower alkyl; or
C—R₁₄ and C—R₁₅ are, independently from each other, replaced by nitrogen;
or a pharmaceutically acceptable salt thereof.

Preferred are the compounds of formula (IB) wherein

C—R₁₄ is replaced by nitrogen;
R₁₅ is hydrogen;
or a pharmaceutically acceptable salt thereof.

Further preferred are the compounds of formula (IB) having the formula

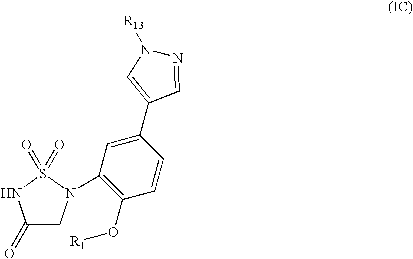

wherein
R₁ is hydrogen, —C(O)R₄, —C(O)NR₅R₆ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₁₃ is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
or a pharmaceutically acceptable salt thereof.

Preferred are the compounds of formula (IC) wherein

R₁₃ is —(CH₂)ₙ—CR₁₆R₁₇—(CH₂)ₘ-Z in which
n and m are, independently from each other, zero or an integer from 1 to 6;
R₁₆ and R₁₇ are, independently from each other, hydrogen or lower alkyl; or
R₁₆ and R₁₇ combined are alkylene which together with the carbon atom to which they are attached form a 3- to 7-membered ring;
Z is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
or a pharmaceutically acceptable salt thereof.

Further preferred are the compounds of formula (IC) wherein

n is an integer from 1 to 3;
m is zero or 1;
R₁₆ and R₁₇ are, independently from each other, hydrogen or lower alkyl;
Z is hydroxy, carbamoyl, cyano, trifluoromethyl, free or esterified carboxy, heterocyclyl, monocyclic aryl or monocyclic aryloxy;
or a pharmaceutically acceptable salt thereof.

More preferred are the compounds of formula (IC) wherein

R₁₆ and R₁₇ are hydrogen;
Z is hydroxy, cyano or free or esterified carboxy;
or a pharmaceutically acceptable salt thereof.

Most preferred are the compounds of formula (IC) wherein

R₁ is hydrogen or —C(O)R₄ in which R₄ is monocyclic aryl;
or a pharmaceutically acceptable salt thereof.

Particular embodiments of the compounds are:
5-[2-Hydroxy-5-(1H-pyrrol-2-yl)-phenyl]-1,1-dioxo-1,2,5-thiadiazolidin-3-one;
5-(4-Hydroxybiphenyl-3-yl)-1,1-dioxo-1,2,5-thiadiazolidin-3-one;
5-[2-Hydroxy-5-(2H-pyrazol-3-yl)-phenyl]-1,1-dioxo-1,2,5-thiadiazolidin-3-one;
5-[2-Hydroxy-5-(1-methyl-1H-pyrazol-4-yl)-phenyl]-1,1-dioxo-1,2,5-thiadiazolidin-3-one;
5-(5-Furan-3-yl-2-hydroxyphenyl)-1,1-dioxo-1,2,5-thiadiazolidin-3-one;
5-[2-Hydroxy-5-(1H-pyrazol-4-yl)-phenyl]-1,1-dioxo-1,2,5-thiadiazolidin-3-one;
5-(4′-Acetyl-4-hydroxybiphenyl-3-yl)-1,1-dioxo-1,2,5-thiadiazolidin-3-one;
5-(4′-Benzoyl-4-hydroxybiphenyl-3-yl)-1,1-dioxo-1,2,5-thiadiazolidin-3-one;
5-[2-Hydroxy-5-(1H-pyrrol-3-yl)-phenyl]-1,1-dioxo-1,2,5-thiadiazolidin-3-one;
Methanesulfonic acid 4′-hydroxy-3′-(1,1,4-trioxo-1,2,5-thiadiazolidin-2-yl)-biphenyl-3-yl ester;
5-(3′-Amino-4-hydroxybiphenyl-3-yl)-1,1-dioxo-1,2,5-thiadiazolidin-3-one;
5-(4-Hydroxy-2′-methylbiphenyl-3-yl)-1,1-dioxo-1,2,5-thiadiazolidin-3-one;
5-[2-Hydroxy-5-(1H-indol-2-yl)-phenyl]-1,1-dioxo-1,2,5-thiadiazolidin-3-one;
[4′-Hydroxy-3′-(1,1,4-trioxo-1,2,5-thiadiazolidin-2-yl)-biphenyl-3-yl]-acetonitrile;
4′-Hydroxy-3′-(1,1,4-trioxo-1,2,5-thiadiazolidin-2-yl)-biphenyl-3-carboxylic acid (2-cyanoethyl)-amide;
3-[4′-Hydroxy-3′-(1,1,4-trioxo-1,2,5-thiadiazolidin-2-yl)-biphenyl-3-yl]-propionic acid methyl ester;
4′-Hydroxy-3′-(1,1,4-trioxo-1,2,5-thiadiazolidin-2-yl)-biphenyl-3-carboxylic acid (2-carbamoylethyl)-amide;
5-[3′-(2-Aminoethyl)-4-hydroxybiphenyl-3-yl]-1,1-dioxo-1,2,5-thiadiazolidin-3-one;
5-(3′-Aminomethyl-4-hydroxybiphenyl-3-yl)-1,1-dioxo-1,2,5-thiadiazolidin-3-one;
5-(2-Hydroxy-5-pyridin-3-yl-phenyl)-1,1-dioxo-1,2,5-thiadiazolidin-3-one;
5-(4-Hydroxy-2′-methoxy-biphenyl-3-yl)-1,1-dioxo-1,2,5-thiadiazolidin-3-one;
5-(2-Hydroxy-5-pyridin-4-yl-phenyl)-1,1-dioxo-1,2,5-thiadiazolidin-3-one;
[4′-Hydroxy-3′-(1,1,4-trioxo-1,2,5-thiadiazolidin-2-yl)-biphenyl-4-yl]-acetic acid;
5-(4′-Chloro-4-hydroxybiphenyl-3-yl)-1,1-dioxo-1,2,5-thiadiazolidin-3-one;
5-(3′-Chloro-4-hydroxybiphenyl-3-yl)-1,1-dioxo-1,2,5-thiadiazolidin-3-one;
5-[2-Hydroxy-5-(6-methoxypyridin-3-yl)-phenyl]-1,1-dioxo-1,2,5-thiadiazolidin-3-one;
5-[5-(6-Fluoropyridin-3-yl)-2-hydroxyphenyl]-1,1-dioxo-1,2,5-thiadiazolidin-3-one;
3-[4′-Hydroxy-3′-(1,1,4-trioxo-1,2,5-thiadiazolidin-2-yl)-biphenyl-3-yl]-propionic acid ethyl ester;
5-(4-Hydroxy-3′-methylbiphenyl-3-yl)-1,1-dioxo-1,2,5-thiadiazolidin-3-one;
5-(3′-Fluoro-4-hydroxybiphenyl-3-yl)-1,1-dioxo-1,2,5-thiadiazolidin-3-one;
5-(4′-Fluoro-4-hydroxybiphenyl-3-yl-1,1-dioxo-1,2,5-thiadiazolidin-3-one;
5-(4-Hydroxy-4′-methylbiphenyl-3-yl)-1,1-dioxo-1,2,5-thiadiazolidin-3-one;
3-[4′-Hydroxy-3′-(1,1,4-trioxo-1,2,5-thiadiazolidin-2-yl)-biphenyl-3-yl]-propionitrile;
4′-Hydroxy-3′-(1,1,4-trioxo-1,2,5-thiadiazolidin-2-yl)-biphenyl-3-carbonitrile;
5-(4-Hydroxy-3′,5′-dimethylbiphenyl-3-yl)-1,1-dioxo-1,2,5-thiadiazolidin-3-one;
5-(4-Hydroxy-3′-methoxybiphenyl-3-yl)-1,1-dioxo-1,2,5-thiadiazolidin-3-one;
N-(2-Hydroxyethyl)-2-[4′-hydroxy-3′-(1,1,4-trioxo-1,2,5-thiadiazolidin-2-yl)-biphenyl-4-yl]-acetamide;
2,2,2-Trifluoro-N-[4′-hydroxy-3′-(1,1,4-trioxo-1,2,5-thiadiazolidin-2-yl)-biphenyl-3-yl]-acetamide;
1-Ethyl-3-[4′-hydroxy-3′-(1,1,4-trioxo-1,2,5-thiadiazolidin-2-yl)-biphenyl-3-yl]-urea;
1-Ethyl-3-[4′-hydroxy-3′-(1,1,4-trioxo-1,2,5-thiadiazolidin-2-yl)-biphenyl-3-ylmethyl]-urea;
[4′-Hydroxy-3′-(1,1,4-trioxo-1,2,5-thiadiazolidin-2-yl)-biphenyl-3-ylmethyl]-carbamic acid methyl ester;
N-[4′-Hydroxy-3′-(1,1,4-trioxo-1,2,5-thiadiazolidin-2-yl)-biphenyl-3-ylmethyl]-acetamide;
[4′-Hydroxy-3′-(1,1,4-trioxo-1,2,5-thiadiazolidin-2-yl)-biphenyl-3-ylmethyl]-carbamic acid benzyl ester;
1-Ethyl-3-[4′-hydroxy-3′-(1,1,4-trioxo-1,2,5-thiadiazolidin-2-yl)-biphenyl-4-yl]-urea;
3-[4′-Hydroxy-3′-(1,1,4-trioxo-1,2,5-thiadiazolidin-2-yl)-biphenyl-3-yl]-propionic acid;
5-{-4-[4-Hydroxy-3-(1,1,4-trioxo-1,2,5-thiadiazolidin-2-yl)-phenyl]-pyrazol-1-yl}-pentanoic acid;
5-[2-Hydroxy-5-(1-propyl-1H-pyrazol-4-yl)-phenyl]-1,1-dioxo-1,2,5-thiadiazolidin-3-one;
5-[2-Hydroxy-5-(1-isobutyl-1H-pyrazol-4-yl)-phenyl]-1,1-dioxo-1,2,5-thiadiazolidin-3-one;
5-{4-[4-Hydroxy-3-(1,1,4-trioxo-1,2,5-thiadiazolidin-2-yl)-phenyl]-1H-pyrazol-1-yl}-pentanoic acid ethyl ester;
5-{2-Hydroxy-5-[1-(4,4,4-trifluorobutyl)-1H-pyrazol-4-yl]-phenyl}-1,1-dioxo-1,2,5-thiadiazolidin-3-one;
5-{2-Hydroxy-5-[1-(3-methylbutyl)-1H-pyrazol-4-yl]-phenyl}-1,1-dioxo-1,2,5-thiadiazolidin-3-one;
5-{4-[4-Hydroxy-3-(1,1,4-trioxo-1,2,5-thiadiazolidin-2-yl)-phenyl]-1H-pyrazol-1-yl}-pentanenitrile;
4-{-4-[4-Hydroxy-3-(1,1,4-trioxo-1,2,5-thiadiazolidin-2-yl)-phenyl]-1H-pyrazol-1-yl}-butyronitrile;
5-(2-Hydroxy-5-phenoxyphenyl)-1,1-dioxo-1,2,5-thiadiazolidin-3-one;
5-(2-Hydroxy-5-methoxyphenyl)-1,1-dioxo-1,2,5-thiadiazolidin-3-one;
5-(5-Benzyl-2-hydroxyphenyl)-1,1-dioxo-1,2,5-thiadiazolidin-3-one;
5-(2-Hydroxy-5-methylphenyl)-1,1-dioxo-1,2,5-thiadiazolidin-3-one;
5-(5-Hexyl-2-hydroxyphenyl)-1,1-dioxo-1,2,5-thiadiazolidin-3-one;
5-(5-Butyl-2-hydroxyphenyl)-1,1-dioxo-1,2,5-thiadiazolidin-3-one;
5-[2-Hydroxy-5-(tetrahydrofuran-3-yl)-phenyl]-1,1-dioxo-1,2,5-thiadiazolidin-3-one;
5-[5-(4-Fluorophenylethynyl)-2-hydroxyphenyl]-1,1-dioxo-1,2,5-thiadiazolidin-3-one;
6-[4-Hydroxy-3-(1,1,4-trioxo-1,2,5-thiadiazolidin-2-yl)-phenyl]-hex-5-ynenitrile;
6-[4-Hydroxy-3-(1,1,4-trioxo-1,2,5-thiadiazolidin-2-yl)-phenyl]-hex-5-ynoic acid;
5-[5-(3,3-Dimethyl-but-1-ynyl)-2-hydroxyphenyl]-1,1-dioxo-1,2,5-thiadiazolidin-3-one;
5-[2-Hydroxy-5-(5-methylhexyl)-phenyl]-1,1-dioxo-1,2,5-thiadiazolidin-3-one;
6-[4-Hydroxy-3-(1,1,4-trioxo-1,2,5-thiadiazolidin-2-yl)-phenyl]-hexanoic acid;
5-[5-(Benzylaminomethyl)-2-hydroxyphenyl]-1,1-dioxo-1,2,5-thiadiazolidin-3-one;
5-(5-Butylaminomethyl-2-hydroxyphenyl)-1,1-dioxo-1,2,5-thiadiazolidin-3-one;
5-{2-Hydroxy-5-[(2-methoxybenzylamino)-methyl]-phenyl}-1,1-dioxo-1,2,5-thiadiazolidin-3-one;
5-{5-[(2-Ethoxybenzylamino)-methyl]-2-hydroxyphenyl}-1,1-dioxo-1,2,5-thiadiazolidin-3-one;
5-{2-Hydroxy-5-[(2-isopropoxybenzylamino)-methyl]-phenyl}-1,1-dioxo-1,2,5-thiadiazolidin-3-one;
5-(2-Hydroxy-5-{[2-(1-methyl-2-phenylethoxy)-benzylamino]-methyl}-phenyl)-1,1-dioxo-1,2,5-thiadiazolidin-3-one;
5-[2-Hydroxy-5-(3-methylbutoxy)-phenyl]-1,1-dioxo-1,2,5-thiadiazolidin-3-one;
5-[2-Hydroxy-5-(4-methylpentyloxy)-phenyl]-1,1-dioxo-1,2,5-thiadiazolidin-3-one;
5-(2-Hydroxy-5-propoxyphenyl)-1,1-dioxo-1,2,5-thiadiazolidin-3-one;
2-Hydroxy-6-{4-[4-hydroxy-3-(1,1,4-trioxo-1,2,5-thiadiazolidin-2-yl)-phenyl]-butoxy}-N,N-dimethylbenzamide;
2-Hydroxy-6-{5-[4-hydroxy-3-(1,1,4-trioxo-1,2,5-thiadiazolidin-2-yl)-phenyl]-pentyloxy}-N,N-dimethylbenzamide;
2-Hydroxy-6-{6-[4-hydroxy-3-(1,1,4-trioxo-1,2,5-thiadiazolidin-2-yl)-phenyl]-hexyloxy}-N,N-dimethylbenzamide;
2-Fluoro-6-{6-[4-hydroxy-3-(1,1,4-trioxo-1,2,5-thiadiazolidin-2-yl)-phenyl]-hexyloxy}-N,N-dimethylbenzamide;
2-Hydroxy-6-{7-[4-hydroxy-3-(1,1,4-trioxo-1,2,5-thiadiazolidin-2-yl)-phenyl]-heptyloxy}-N,N-dimethylbenzamide;
5-(4-Hydroxy-4′-hydroxymethylbiphenyl-3-yl)-1,1-dioxo-1,2,5-thiadiazolidin-3-one;
5-(2-Hydroxy-4,5-dimethylphenyl)-1,1-dioxo-1,2,5-thiadiazolidin-3-one;
5-[4-Hydroxy-3-(1,1,4-trioxo-1,2,5-thiadiazolidin-2-yl)-phenyl]-2,2-dimethylpentanoic acid;
8-[4-Hydroxy-3-(1,1,4-trioxo-1,2,5-thiadiazolidin-2-yl)-phenyl]-2,2-dimethyloctanoic acid ethyl ester;
8-[4-Hydroxy-3-(1,1,4-trioxo-1,2,5-thiadiazolidin-2-yl)-phenyl]-2,2-dimethyloctanoic acid;
7-[4-Hydroxy-3-(1,1,4-trioxo-1,2,5-thiadiazolidin-2-yl)-phenyl]-2,2-dimethylheptanoic acid;
6-[4-Hydroxy-3-(1,1,4-trioxo-1,2,5-thiadiazolidin-2-yl)-phenyl]-2,2-dimethylhexanoic acid;
7-[4-Hydroxy-3-(1,1,4-trioxo-1,2,5-thiadiazolidin-2-yl)-phenyl]-2,2-dimethylheptanoic acid ethyl ester;
8-[4-Hydroxy-3-(1,1,4-trioxo-1,2,5-thiadiazolidin-2-yl)-phenyl]-2,2-dimethyloctanenitrile;
5-[2-Hydroxy-5-(6-hydroxy-6-methylheptyl)-phenyl]-1,1-dioxo-1,2,5-thiadiazolidin-3-one;
5-[2-Hydroxy-5-(7-hydroxy-6,6-dimethylheptyl)-phenyl]-1,1-dioxo-1,2,5-thiadiazolidin-3-one;
5-[2-Hydroxy-5-(5-hydroxy-5-methylhexyl)-phenyl]-1,1-dioxo-1,2,5-thiadiazolidin-3-one;
5-[2-hydroxy-5-(8-hydroxy-7,7-dimethyloctyl)-phenyl]-1,1-dioxo-1,2,5-thiadiazolidin-3-one;
7-[4-Hydroxy-3-(1,1,4-trioxo-1,2,5-thiadiazolidin-2-yl)-phenyl]-2,2-dimethylheptanenitrile;
5-[2-Hydroxy-5-(5-hydroxy-5-methylhex-1-ynyl)-phenyl]-1,1-dioxo-1,2,5-thiadiazolidin-3-one;
5-[2-Hydroxy-5-(2-pyridin-3-yl-ethyl)-phenyl]-1,1-dioxo-1,2,5-thiadiazolidin-3-one;
5-(2-Hydroxy-4-methyl-5-pentylphenyl)-1,1-dioxo-1,2,5-thiadiazolidin-3-one;
5-(2-Hydroxy-4-methyl-5-propylphenyl)-1,1-dioxo-1,2,5-thiadiazolidin-3-one;
5-(5-Heptyl-2-hydroxy-4-methylphenyl)-1,1-dioxo-1,2,5-thiadiazolidin-3-one;
5-[5-(2-Cyclohexylethyl)-2-hydroxy-4-methylphenyl]-1,1-dioxo-1,2,5-thiadiazolidin-3-one;
Benzoic acid 4-(7-hydroxy-6,6-dimethylheptyl)-2-(1,1,4-trioxo-1,2,5-thiadiazolidin-2-yl)-phenyl ester; and
Benzoic acid 4-(6-cyano-6,6-dimethylhexyl)-2-(1,1,4-trioxo-1,2,5-thiadiazolidin-2-yl)-phenylester;
   or a pharmaceutically acceptable salt thereof.

### Category 4 PTP Inhibitors

The methods of the invention can be practiced with the compounds of the formula

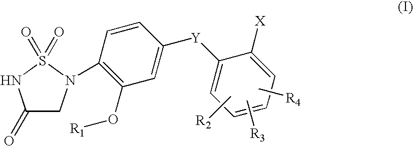

wherein
R₁ is hydrogen, —C(O)R₅, —C(O)NR₆R₇ or —C(O)OR₈ in which
R₅ and R₆ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₇ and R₈ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂, R₃ and R₄ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
R₂ and R₃ combined are alkylene which together with the ring atoms to which they are attached form a 5- to 7-membered fused ring provided R₂ and R₃ are attached to carbon atoms adjacent to each other; or
R₂ and R₃ combined together with the carbon atom to which they are attached form a fused 5- to 6-membered aromatic or heteroaromatic ring provided R₂ and R₃ are attached to carbon atoms adjacent to each other;
X is hydrogen, fluoro, cyano, or free or esterified carboxy; or
X is —NR₉C(O)R₁₀, —NR₈C(O)OR₁₁, —NR₈S(O)₂R₁₂, —(CH₂)ₘS(O)₂R₁₃, —OS(O)₂R₁₄ or —OₙC(O)NR₁₅R₁₈ in which
R₉ is hydrogen, lower alkyl, acyl, alkoxycarbonyl or sulfonyl;
   R₁₀, R₁₁, R₁₂, R₁₃ and R₁₄ are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₁₀, R₁₂ and R₁₃ are, independently from each other, —NR₁₇R₁₈ in which
   R₁₇ and R₁₈ are, independently from each other, hydrogen, alkyl, cycloalkyl, aralkyl, aryl or heterocyclyl; or
   R₁₇ and R₁₈ combined are alkylene which together with the nitrogen atom to which they are attached form a 4- to 7-membered ring;
   R₁₅ and R₁₆ are, independently from each other, hydrogen, alkyl, cycloalkyl, aralkyl, aryl or heterocyclyl; or
   R₁₅ and R₁₆ combined are alkylene which together with the nitrogen atom to which they are attached form a 4- to 7-membered ring;
   m and n are, independently from each other, zero or an integer of 1; or
   C—X is replaced by nitrogen;

### Y is CH₂, O or S;

or a pharmaceutically acceptable salt thereof.

Preferred are the compounds of formula (I) wherein

### Y is CH₂;

or a pharmaceutically acceptable salt thereof.

Further preferred are the compounds of formula (I) having the formula

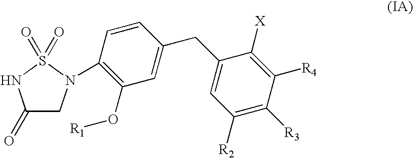

wherein
R₁ is hydrogen, —C(O)R₅, —C(O)NR₆R₇ or —C(O)OR₈ in which
R₅ and R₆ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₇ and R₈ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂, R₃ and R₄ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
R₂ and R₃ combined are alkylene which together with the ring atoms to which they are attached form a 5- to 7-membered fused ring; or
R₂ and R₃ combined together with the carbon atom to which they are attached form a fused 5- to 6-membered aromatic or heteroaromatic ring;
X is cyano; or
X is —NR₉C(O)R₁₀, —NR₉C(O)OR₁₁, —NR₉S(O)₂R₁₂, —(CH₂)ₘS(O)₂R₁₃ or —OS(O)₂R₁₄ in which
R₉ is hydrogen or lower alkyl;
   R₁₀, R₁₁, R₁₂, R₁₃ and R₁₄ are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₁₀, R₁₂ and R₁₃ are, independently from each other, —NR₁₇R₁₈ in which
   R₁₇ and R₁₈ are, independently from each other, hydrogen, alkyl, cycloalkyl, aralkyl, aryl or heterocyclyl; or
   R₁₇ and R₁₈ combined are alkylene which together with the nitrogen atom to which they are attached form a 4- to 7-membered ring;
   m is zero; or
   C—X is replaced by nitrogen;
   or a pharmaceutically acceptable salt thereof.

Preferred are the compounds of formula (IA) wherein

X is cyano; or
X is —NR₉S(O)₂R₁₂ or —OS(O)₂R₁₄ in which
R₉ is hydrogen or lower alkyl;
R₁₂ and R₁₄ are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
or a pharmaceutically acceptable salt thereof.

Especially preferred are the compounds of formula (IA), designated as the A group, wherein

R₉ is hydrogen;
or a pharmaceutically acceptable salt thereof.

Preferred are the compounds in the A group wherein

R₁₂ and R₁₄ are, independently from each other, monocyclic aryl or C₍₁₋₄₎alkyl;
or a pharmaceutically acceptable salt thereof.

Further preferred are the compounds in the A group wherein

R₁ is hydrogen or —C(O)R₅ in which R₅ is monocyclic aryl;
or a pharmaceutically acceptable salt thereof.

Especially preferred are also the compounds of formula (IA), designated as the B group, wherein

R₂, R₃ and R₄ are, independently from each other, hydrogen, halogen, hydroxy, monocyclic aryl, C₍₁₋₄₎alkoxy or C₍₁₋₄₎alkyl optionally substituted with at least one halogen;
or a pharmaceutically acceptable salt thereof.

Preferred are the compounds in the B group wherein

R₉ is hydrogen;
or a pharmaceutically acceptable salt thereof.

Further preferred are the compounds in the B group wherein

R₁₂ and R₁₄ are, independently from each other, monocyclic aryl or C₍₁₋₄₎alkyl;
or a pharmaceutically acceptable salt thereof.

More preferred are the compounds in the B group wherein

R₁ is hydrogen or —C(O)R₅ in which R₅ is monocyclic aryl;
or a pharmaceutically acceptable salt thereof.

Particular embodiments of the compounds are:
5-(4-Benzyl-2-hydroxyphenyl)-1,1-dioxo-1,2,5-thiadiazolidin-3-one;
5-[2-Hydroxy-4-(3-hydroxybenzyl)-phenyl]-1,1-dioxo-1,2,5-thiadiazolidin-3-one;
5-[2-Hydroxy-4-(3-methoxybenzyl)-phenyl]-1,1-dioxo-1,2,5-thiadiazolidin-3-one;
5-[4-(2-Fluoro-3-trifluoromethylbenzyl)-2-hydroxyphenyl]-1,1-dioxo-1,2,5-thiadiazolidin-3-one;
2-[3-Hydroxy-4-(1,1,4-trioxo-1,2,5-thiadiazolidin-2-yl)-benzyl]-benzonitrile;
5-[4-(2-Fluorobenzyl)-2-hydroxyphenyl]-1,1-dioxo-1,2,5-thiadiazolidin-3-one;
5-(2-Hydroxy-4-naphthalen-2-ylmethylphenyl)-1,1-dioxo-1,2,5-thiadiazolidin-3-one;
5-[2-Hydroxy-4-(3-trifluoromethylbenzylphenyl]-1,1-dioxo-1,2,5-thiadiazolidin-3-one;
5-[2-Hydroxy-4-(2-methylbenzyl)phenyl]-1,1-dioxo-1,2,5-thiadiazolidin-3-one;
5-[4-(4-Fluorobenzyl)-2-hydroxyphenyl]-1,1-dioxo-1,2,5-thiadiazolidin-3-one;
3-[3-Hydroxy-4-(1,1,4-trioxo-1,2,5-thiadiazolidin-2-yl)-benzyl]-benzoic acid methyl ester;
5-(4-Biphenyl-3-ylmethyl-2-hydroxyphenyl)-1,1-dioxo-1,2,5-thiadiazolidin-3-one;
5-[4-(3-Fluoro-4-methylbenzyl)-2-hydroxyphenyl]-1,1-dioxo-1,2,5-thiadiazolidin-3-one;
5-[2-Hydroxy-4-(4-methylbenzyl)phenyl]-1,1-dioxo-1,2,5-thiadiazolidin-3-one;
5-[2-Hydroxy-4-(4-hydroxybenzyl)phenyl]-1,1-dioxo-1,2,5-thiadiazolidin-3-one;
5-[4-(3-Fluorobenzyl)-2-hydroxyphenyl]-1,1-dioxo-1,2,5-thiadiazolidin-3-one;
5-[4-(4-tert-Butylbenzyl)-2-hydroxyphenyl]-1,1-dioxo-1,2,5-thiadiazolidin-3-one;
5-[4-{2-Benzenesulfonylmethylbenzyl)-2-hydroxyphenyl]-1,1-dioxo-1,2,5-thiadiazolidin-3-one;
5-[2-Hydroxy-4-(3-methylbenzyl)phenyl]-1,1-dioxo-1,2,5-thiadiazolidin-3-one;
{2-[3-Hydroxy-4-(1,1,4-trioxo-1,2,5-thiadiazolidin-2-yl)-benzyl]-phenyl}-carbamic acid tert-butyl ester;
N-{2-[3-Hydroxy-4-(1,1,4-trioxo-1,2,5-thiadiazolidin-2-yl)-benzyl]-phenyl}-C-phenyl-methanesulfonamide;
N-{2-[3-Hydroxy-4-(1,1,4-trioxo-1,2,5-thiadiazolidin-2-yl)-benzyl]-phenyl}-benzenesulfonamide;
Ethanesulfonic acid {2-[3-hydroxy-4-(1,1,4-trioxo-1,2,5-thiadiazolidin-2-yl)-benzyl]-phenyl}-amide;
Propane-1-sulfonic acid {2-[3-hydroxy-4-(1,1,4-trioxo-1,2,5-thiadiazolidin-2-yl)-benzyl]-phenyl}-amide;
Butane-1-sulfonic acid {2-[3-hydroxy-4-(1,1,4-trioxo-1,2,5-thiadiazolidin-2-yl)-benzyl]-phenyl}-amide;
C-Cyclohexyl-N-{2-[3-hydroxy-4-(1,1,4-trioxo-1,2,5-thiadiazolidin-2-yl)-benzyl]-phenyl}-methanesulfonamide;
N-{2-[3-Hydroxy-4-(1,1,4-trioxo-1,2,5-thiadiazolidin-2-yl)-benzyl]-phenyl}-methanesulfonamide;
N-{2-[3-Hydroxy-4-(1,1,4-trioxo-1,2,5-thiadiazolidin-2-yl)-benzyl]-phenyl}-4-isopropylbenzenesulfonamide;
N-{2-[3-Hydroxy-4-(1,1,4-trioxo-1,2,5-thiadiazolidin-2-yl)-benzyl]-phenyl}-aminosulfonamide;
N-{3-[3-Hydroxy-4-(1,1,4-trioxo-1,2,5-thiadiazolidin-2-yl)-benzyl]-naphthalen-2-yl}-methanesulfonamide;
N-{2-[3-Hydroxy-4-(1,1,4-trioxo-1,2,5-thiadazolidin-2-yl)-benzyl]-phenyl}-acetamide;
4-tert-Butyl-N-{2-[3-hydroxy-4-(1,1,4-trioxo-1,2,5-thiadiazolidin-2-yl)-benzyl]-phenyl}-benzamide;
N-{2-[3-Hydroxy-4-(1,1,4-trioxo-1,2,5-thiadiazolidin-2-yl)-benzyl]-phenyl}-benzamide;
5-[4-(4-Ethylpyridin-2-ylmethyl)-2-hydroxyphenyl]-1,1-dioxo-1,2,5-thiadiazolidin-3-one;
5-[4-(6-Methoxypyridin-2-ylmethyl)-2-hydroxyphenyl]-1,1-dioxo-1,2,5-thiadiazolidin-3-one;
5-(2-Hydroxy-4-pyridin-2-ylmethylphenyl)-1,1-dioxo-1,2,5-thiadiazolidin-3-one;
5-[2-Hydroxy-4-(2-methanesulfonylbenzyl)-phenyl]-1,1-dioxo-1,2,5-thiadiazolidin-3-one;
N-{2-[3-Hydroxy-4-(1,1,4-trioxo-1,2,5-thiadiazolidin-2-yl)-benzyl]-phenyl}-N-methylmethanesulfonamide;
5-[2-Hydroxy-4-(2-methanesulfonylmethylbenzyl)-phenyl]-1,1-dioxo-1,2,5-thiadiazolidin-3-one;
5-{4-(3-Methansulfonylphenyl)methyl-2-hydroxyphenyl}-1,1-dioxo-1,2,5-thiadiazolidin-3-one;
C-{2-[3-Hydroxy-4-(1,1,4-trioxo-1,2,5-thiadiazolidin-2-yl)-benzyl]-phenyl}-N,N-dimethylmethanesulfonamide;
Methanesulfonic acid 2-[3-hydroxy-4-(1,1,4-trioxo-1,2,5-thiadiazoldin-2-yl)-benzyl]-phenyl ester;
Methanesulfonic acid 3-[3-hydroxy-4-(1,1,4-trioxo-1,2,5-thiadiazolidin-2-yl)-benzyl]-naphthalen-2-yl ester;
Methanesulfonic acid 2-[3-hydroxy-4-(1,1,4-trioxo-1,2,5-thiadiazolidin-2-yl)-benzyl]-naphthalen-1-yl ester;
Methanesulfonic acid 2-[3-hydroxy-4-(1,1,4-trioxo-1,2,5-thiadiazolidin-2-yl)-benzyl]-4-methylphenyl ester;
Methanesulfonic acid 1-[3-hydroxy-4-(1,1,4-trioxo-1,2,5-thiadiazolidin-2-yl)-benzyl]-naphthalen-2-yl ester;
Methanesulfonic acid 2-[3-hydroxy-4-(1,1,4-trioxo-1,2,5-thiadiazolidin-2-yl)-benzyl]-4-methoxyphenyl ester;
Methanesulfonic acid 2-[3-hydroxy-4-(1,1,4-trioxo-1,2,5-thiadiazolidin-2-yl)-benzyl]-6-methylphenyl ester;
Ethanesulfonic acid 2-[3-hydroxy-4-(1,1,4-trioxo-1,2,5-thiadiazolidin-2-yl)-benzyl]-4-methylphenyl ester;
Propane-1-sulfonic acid 2-[3-hydroxy-4-(1,1,4-trioxo-1,2,5-thiadiazolidin-2-yl)-benzyl]-4-methylphenyl ester;
Methanesulfonic acid 4-chloro-2-[3-hydroxy-4-(1,1,4-trioxo-1,2,5-thiadiazolidin-2-yl)-benzyl]-phenyl ester;
Methanesulfonic acid 2-[3-hydroxy-4-(1,1,4-trioxo-1,2,5-thiadiazolidin-2-yl)-benzyl]-5-methylphenyl ester;
Methanesulfonic acid 4-chloro-2-[3-hydroxy-4-(1,1,4-trioxo-1,2,5-thiadiazolidin-2-yl)-benzyl]-6-methylphenyl ester;
Ethanesulfonic acid 2-[3-hydroxy-4-(1,1,4-trioxo-1,2,5-thiadiazolidin-2-yl)-benzyl]-phenyl ester;
Propane-1-sulfonic acid 2-[3-hydroxy-4-(1,1,4-trioxo-1,2,5-thiadiazolidin-2-yl)-benzyl]-phenyl ester;
5-[4-(2-Fluoro-4-methylbenzyl)-2-hydroxyphenyl]-1,1-dioxo-1,2,5-thiadiazolidin-3-one;
3-[3-Hydroxy-4-(1,1,4-trioxo-1,2,5-thiadiazolidin-2-yl)-benzyl]-N-methylbenzamide potassium salt;
3-[3-Hydroxy-4-(1,1,4-trioxo-1,2,5-thiadiazolidin-2-yl)-benzyl]-benzoic acid dipotassium salt;
2-[3-Hydroxy-4-(1,1,4-trioxo-1,2,5-thiadiazolidin-2-yl)-benzyl]-benzoic acid;
5-[4-(2,5-Difluorobenzyl)-2-hydroxyphenyl]-1,1-dioxo-1,2,5-thiadiazolidin-3-one;
5-[4-(3-Ethylbenzyl)-2-hydroxyphenyl]-1,1-dioxo-1,2,5-thiadiazolidin-3-one;
5-(2-Hydroxy-4-phenoxyphenyl)-1,1-dioxo-1,2,5-thiadiazolidin-3-one potassium salt;
2-Hydroxy-6-[3-hydroxy-4-(1,1,4-trioxo-1,2,5-thiadiazolidin-2-yl)-benzyl]-benzonitrile;
2-[3-Hydroxy-4-(1,1,4-trioxo-1,2,5-thiadiazolidin-2-yl)-benzyl]-4-trifluoromethylbenzonitrile;
2-[3-Hydroxy-4-(1,1,4-trioxo-1,2,5-thiadiazolidin-2-yl)-benzyl]-4-methylbenzonitrile;
2-[3-Hydroxy-4-(1,1,4-trioxo-1,2,5-thiadiazolidin-2-yl)-benzyl]-6-methyl-benzonitrile;
2-[3-Hydroxy-4-(1,1,4-trioxo-1,2,5-thiadiazolidin-2-yl)-benzyl]-4-trifluoromethylbenzonitrile;
5-(2-Hydroxy-4-phenylsulfanylphenyl)-1,1-dioxo-1,2,5-thiadiazolidin-3-one;
2-[3-Hydroxy-4-(1,1,4-trioxo-1,2,5-thiadiazolidin-2-yl)-phenylsulfanyl]-4-trifluoromethylbenzonitrile;
2-[3-Hydroxy-4-(1,1,4-trioxo-1,2,5-thiadiazolidin-2-yl)-phenylsulfanyl]-6-trifluoromethylbenzonitrile;
Methanesulfonic acid 2-[3-diethylcarbamoyloxy-4-(1,1,4-trioxo-1,2,5-thiadiazolidin-2-yl)-benzyl]-phenyl ester;
Methanesulfonic acid 2-[3-isopropoxycarbonyloxy-4-(1,1,4-trioxo-1,2,5-thiadiazolidin-2-yl)-benzyl]-phenyl ester;
N-{4-Chloro-2-[3-hydroxy-4-(1,1,4-trioxo-1,2,5-thiadiazolidin-2-yl)-benzyl]-phenyl}-methanesulfonamide;
N-{2-[3-Hydroxy-4-(1,1,4-trioxo-1,2,5-thiadiazolidin-2-yl)-benzyl]-4-methylphenyl}-methanesulfonamide;
N-{4-Fluoro-2-[3-hydroxy-4-(1,1,4-trioxo-1,2,5-thiadiazolidin-2-yl)-benzyl]-phenyl}-methanesulfonamide;
N-{4-Fluoro-2-[3-hydroxy-4-(1,1,4-trioxo-1,2,5-thiadiazolidin-2-yl)-benzyl]-phenyl}-benzenesulfonamide;
Ethanesulfonic acid {4-fluoro-2-[3-hydroxy-4-(1,1,4-trioxo-1,2,5-thiadiazolidin-2-yl)-benzyl]-phenyl}-amide;
Propane-2-sulfonic acid {4-fluoro-2-[3-hydroxy-4-(1,1,4-trioxo-1,2,5-thiadiazolidin-2-yl)-benzyl]-phenyl}-amide;
Propane-1-sulfonic acid {2-[3-hydroxy-4-(1,1,4-trioxo-1,2,5-thiadiazolidin-2-yl)-benzyl]-4-methylphenyl}-amide;
N-{4-Fluoro-2-[3-hydroxy-4-(1,1,4-trioxo-1,2,5-thiadiazolidin-2-yl)-benzyl]-phenyl}-C-phenyl-methanesulfonamide;
Ethanesulfonic acid {4-chloro-2-[3-hydroxy-4-(1,1,4-trioxo-1,2,5-thiadiazolidin-2-yl)-benzyl]-phenyl}-amide;
Propane-2-sulfonic acid {4-chloro-2-[3-hydroxy-4-(1,1,4-trioxo-1,2,5-thiadiazolidin-2-yl)-benzyl]-phenyl}-amide;
Propane-1-sulfonic acid {4-chloro-2-[3-hydroxy-4-(1,1,4-trioxo-1,2,5-thiadiazolidin-2-yl)-benzyl]-phenyl}-amide;
Ethanesulfonic acid {4-chloro-2-[3-hydroxy-4-(1,1,4-trioxo-1,2,5-thiadiazolidin-2-yl)-benzyl]-6-methylphenyl}-amide;
N-{2-[3-Hydroxy-4-(1,1,4-trioxo-1,2,5-thiadiazolidin-2-yl)-benzyl]-6-methylphenyl}-methanesulfonamide;
N-{2-[3-Hydroxy-4-(1,1,4-trioxo-1,2,5-thiadiazolidin-2-yl)-benzyl]-4,6-dimethylphenyl}-methanesulfonamide;
Ethanesulfonic acid {2-[3-hydroxy-4-(1,1,4-trioxo-1,2,5-thiadiazolidin-2-yl)-benzyl]-6-methylphenyl}-amide;
Propane-1-sulfonic acid {2-[3-hydroxy-4-(1,1,4-trioxo-1,2,5-thiadiazolidin-2-yl)-benzyl]-6-methylphenyl}-amide;
Ethanesulfonic acid {2-[3-hydroxy-4-(1,1,4-trioxo-1,2,5-thiadiazolidin-2-yl)-benzyl]-4,6-dimethylphenyl}-amide;
N-{4-Chloro-2-[3-hydroxy-4-(1,1,4-trioxo-1,2,5-thiadiazolidin-2-yl)-benzyl]-6-methylphenyl}-methanesulfonamide;
N-{4-Chloro-2-[3-hydroxy-4-(1,1,4-trioxo-1,2,5-thiadiazolidin-2-yl)-benzyl]-6-methylphenyl}-methanesulfonamide;
N-{2-[3-Hydroxy-4-(1,1,4-trioxo-1,2,5-thiadiazolidin-2-yl)-benzyl]-5-methylphenyl}-methanesulfonamide;
N-{2-[3-Hydroxy-4-(1,1,4-trioxo-1,2,5-thiadiazolidin-2-yl)-benzyl]-6-methoxyphenyl}-methanesulfonamide;
N-{5-Chloro-2-[3-hydroxy-4-(1,1,4-trioxo-1,2,5-thiadiazolidin-2-yl)-benzyl]-phenyl}-methanesulfonamide;
Ethanesulfonic acid {2-[3-hydroxy-4-(1,1,4-trioxo-1,2,5-thiadiazolidin-2-yl)-benzyl]-4-methylphenyl}-amide;
Methanesulfonic acid 4-ethyl-2-[3-hydroxy-4-(1,1,4-trioxo-1,2,5-thiadiazolidin-2-yl)-benzyl]-phenyl ester;
Methanesulfonic acid 4-tert-butyl-2-[3-hydroxy-4-(1,1,4-trioxo-1,2,5-thiadiazolidin-2-yl)-benzyl]-phenyl ester;
Diethylcarbamic acid 2-[3-hydroxy-4-(1,1,4-trioxo-1,2,5-thiadiazolidin-2-yl)-benzyl]-4-methylphenyl ester;
Ethanesulfonic acid {4-ethyl-2-[3-hydroxy-4-(1,1,4-trioxo-1,2,5-thiadiazolidin-2-yl)-benzyl]-phenyl}-amide;
Propane-1-sulfonic acid {4-ethyl-2-[3-hydroxy-4-(1,1,4-trioxo-1,2,5-thiadiazolidin-2-yl)-benzyl]-phenyl}-amide;
N-{4-Ethyl-2-[3-hydroxy-4-(1,1,4-trioxo-1,2,5-thiadiazolidin-2-yl)-benzyl]-phenyl}-methanesulfonamide;
N-{4-Benzyl-2-[3-hydroxy-4-(1,1,4-trioxo-1,2,5-thiadiazolidin-2-yl)-benzyl-]-phenyl}methanesulfonamide;
N-{3-[3-Hydroxy-4-(1,1,4-trioxo-1,2,5-thiadiazolidin-2-yl)-benzyl]-biphenyl-4-yl}-methanesulfonamide;
N-{2-[3-Hydroxy-4-(1,1,4-trioxo-1,2,5-thiadiazolidin-2-yl)-benzyl]-4-methoxyphenyl}-methanesulfonamide;
Ethanesulfonic acid {2-[3-hydroxy-4-(1,1,4-trioxo-1,2,5-thiadiazolidin-2-yl)-benzyl]-4-methoxyphenyl}-amide;
Propane-1-sulfonic acid {2-[3-hydroxy-4-(1,1,4-trioxo-1,2,5-thiadiazolidin-2-yl)-benzyl]-4-methoxyphenyl}-amide;
Methanesulfonic acid 5-[3-hydroxy-4-(1,1,4-trioxo-1,2,5-thiadiazolidin-2-yl)-benzyl]-7-methylindan-4-yl ester;
Methanesulfonic acid 6-[3-hydroxy-4-(1,1,4-trioxo-1,2,5-thiadiazolidin-2-yl)-benzyl]-indan-5-yl ester;
N-{2-[4-(1,1-dioxido-4-oxo-1,2,5-thiadiazolidin-2-yl)-3-hydroxybenzyl]-1,4-dimethylphenyl}sulfamide;
N-{2-[4-(1,1-dioxido-4-oxo-1,2,5-thiadiazolidin-2-yl)-3-hydroxybenzyl]-1-methyl-4-chlorophenyl}sulfamide;
N-{2-[4-(1,1-dioxido-4-oxo-1,2,5-thiadiazolidin-2-yl)-3-hydroxybenzyl]-4-ethylphenyl}sulfamide;
Methanesulfonic acid 2-[3-hydroxy-4-(1,1,4-trioxo-1,2,5-thiadiazolidin-2-yl)-benzyl]-6-isopropylphenyl ester;
Methanesulfonic acid 2-[3-hydroxy-4-(1,1,4-trioxo-1,2,5-thiadiazolidin-2-yl)-benzyl]-5-methylphenyl ester;
Methanesulfonic acid 2-chloro-6-[3-hydroxy-4-(1,1,4-trioxo-1,2,5-thiadiazolidin-2-yl)-benzyl]-phenyl ester;
Methanesulfonic acid 5-chloro-2-[3-hydroxy-4-(1,1,4-trioxo-1,2,5-thiadiazolidin-2-yl)-benzyl]-phenyl ester;
Methanesulfonic acid 2-[3-hydroxy-4-(1,1,4-trioxo-1,2,5-thiadiazolidin-2-yl)-benzyl]-5-methoxyphenyl ester;
Methanesulfonic acid 2-[3-hydroxy-4-(1,1,4-trioxo-1,2,5-thiadiazolidin-2-yl)-benzyl]-6-methoxyphenyl ester;
N-{2-Chloro-6-[3-hydroxy-4-(1,1,4-trioxo-1,2,5-thiadiazolidin-2-yl)-benzyl]-phenyl}-methanesulfonamide;
Methanesulfonic acid 2-[3-hydroxy-4-(1,1,4-trioxo-1,2,5-thiadiazolidin-2-yl)-benzyl]-4,6-dimethylphenyl ester;
Benzoic acid 5-benzyl-2-(1,1,4-trioxo-1,2,5-thiadiazolidin-2-yl)-phenyl ester;
Benzoic acid 5-(2-methanesulfonyloxybenzyl)-2-(1,1,4-trioxo-1,2,5-thiadiazolidin-2-yl)-phenyl ester;
Benzoic acid 5-(2-methanesulfonyloxy-5-methylbenzyl)-2-(1,1,4-trioxo-1,2,5-thiadiazolidin-2-yl)-phenyl ester;
Benzoic acid 5-(2-methanesulfonylamino-5-methylbenzyl)-2-(1,1,4-trioxo-1,2,5-thiadiazolidin-2-yl)-phenyl ester;
Benzoic acid 5-(2-methanesulfonylaminobenzyl)-2-(1,1,4-trioxo-1,2,5-thiadiazolidin-2-yl)-phenyl ester;
Benzoic acid 5-[2-(benzoylmethanesulfonylamino)-5-methylbenzyl]-2-(1,1,4-trioxo-1,2,5-thiadiazolidin-2-yl)-phenyl ester;
Benzoic acid 5-[2-(benzoylmethanesulfonylamino)-benzyl]-2-(1,1,4-trioxo-1,2,5-thiadiazolidin-2-yl)-phenyl ester;
2-Amino-3-methylbutyric acid 5-(2-methanesulfonyloxy-benzyl)-2-(1,1,4-trioxo-1,2,5-thiadiazolidin-2-yl)-phenyl ester;
Benzoic acid 5-(5-chloro-2-methanesulfonylamino-3-methylbenzyl)-2-(1,1,4-trioxo-1,2,5-thiadiazolidin-2-yl)-phenyl ester;
Benzoic acid 5-(2-methanesulfonylamino-3,5-dimethylbenzyl)-2-(1,1,4-trioxo-1,2,5-thiadiazolidin-2-yl)-phenyl ester;
2-Amino-3-methylbutyric acid 5-(2-methanesulfonyloxy-5-methylbenzyl-2-(1,1,4-trioxo-1,2,5-thiadiazolidin-2-yl)-phenyl ester;
Benzoic acid 5-(2-methanesulfonyloxy-3,5-dimethylbenzyl)-2-(1,1,4-trioxo-1,2,5-thiadiazolidin-2-yl)-phenyl ester;
Methanesulfonic acid 2-[3-methoxycarbonyloxy-4-(1,1,4-trioxo-1,2,5-thiadiazolidin-2-yl)-benzyl]-4-methylphenyl ester;
2-Amino-3-methylbutyric acid 5-(2-methanesulfonylamino-benzyl)-2-(1,1,4-trioxo-1,2,5-thiadiazolidin-2-yl)-phenyl ester;
2-(1,1-dioxido-4-oxo-1,2,5-thiadiazolidin-2-yl)-5-{2-[(methoxycarbonyl)(methylsulfonyl)-amino]-3,5-dimethylbenzyl}phenyl methyl carbonate;
Carbonic acid 5-(2-methanesulfonylamino-3,5-dimethylbenzyl)-2-(1,1,4-trioxo-1,2,5-thiadiazolidin-2-yl)-phenyl ester methyl ester;
Benzoic acid 5-(2-methanesulfonylamino-4-methylbenzyl)-2-(1,1,4-trioxo-1,2,5-thiadiazolidin-2-yl)-phenyl ester;
Benzoic acid 5-(2-methanesulfonyloxy-4-methylbenzyl)-2-(1,1,4-trioxo-1,2,5-thiadiazolidin-2-yl)-phenyl ester;
Benzoic acid 5-[2-(benzoylmethanesulfonylamino)-4-methylbenzyl]-2-(1,1,4-trioxo-1,2,5-thiadiazolidin-2-yl)-phenyl ester;
Benzoic acid 5-(2-methanesulfonyloxy-3-methylbenzyl)-2-(1,1,4-trioxo-1,2,5-thiadiazolidin-2-yl)-phenyl ester;
Benzoic acid 5-(5-chloro-2-methanesulfonyloxy-3-methylbenzyl)-2-(1,1,4-trioxo-1,2,5-thiadiazolidin-2-yl)-phenyl ester;
Benzoic acid 5-[2-(benzoylmethanesulfonylamino)-3-methylbenzyl]-2-(1,1,4-trioxo-1,2,5-thiadiazolidin-2-yl)-phenyl ester;
Benzoic acid 5-(2-methanesulfonylamino-3-methylbenzyl)-2-(1,1,4-trioxo-1,2,5-thiadiazolidin-2-yl)-phenyl ester;
2-Methylbenzoic acid 5-(2-methanesulfonyloxy-5-methylbenzyl)-2-(1,1,4-trioxo-1,2,5-thiadiazolidin-2-yl)-phenyl ester; and
5-(4-Benzyl-2-hydroxy-6-methylphenyl)-1,1-dioxo-1,2,5-thiadiazolidin-3-one;
   or a pharmaceutically acceptable salt thereof.

### Category 5 PTP Inhibitors

The methods of the invention can be practiced with the compounds of the formula

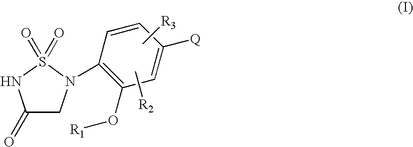

wherein

### Q is:

### i) —X, or

ii)-Y—(CH₂)ₙ—(CR₈R₉)ₚ—(CH₂)ₘ-Z-X in which
   Y is oxygen or S(O)_{q} in which q is zero or an integer of 1 or 2; or

### Y is —C≡C— or —C═C—; or

Y is cyclopropyl or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
R₈ and R₉ combined are alkylene which together with the carbon atom to which they are attached form a 3- to 7-membered ring;
p is zero or an integer selected from 1 or 2
Z is absent;

### Z is —C(O)—O—; or

### Z is —C(O)—; or

Z is —C(O)—NRα-alkylene- or —C(O)—NRα-alkylene-O—, wherein Rα is H or lower alkyl; or
Z is —CO—NRα-(CH₂)_{n′}—(CR_{8′}R_{9′})_{p′}—(CH₂)_{m′}—, or —C(O)—NRα-(CH₂)_{n′}—(CR_{8′}R_{9′})_{p′}—(CH₂)_{m′}—O—, wherein p′ is zero or an integer of 1, n′ and m′ are, independently from each other, zero or an integer from 1 to 8, R_{8′} and R_{9′} are, independently from each other, hydrogen or lower alkyl, Rα is H or lower alkyl; or
Z is —NRα′-C(O)—, or —NRα′-C(O)—O—, wherein Rα′ is H or lower alkyl, or Rα′ and R₉ combined are alkylene which together with the carbon atom to which they are attached form a 3- to 7-membered ring; or

### Z is —C(O)—NH—NH—C(O)—O—; or

### Z is —S(O)₂—, or —S(O)—; or

Z is —NRβ-S(O)₂—, wherein Rβ is H, lower alkyl, or Rβ and R₉ combined are alkylene which together with the carbon atom to which they are attached form a 3- to 7-membered ring; or

### Z is —NH—S(O)₂—NH—C(O)—O—; or

Z is —NRγ-C(O)—NRγ′-; wherein Rγ′ is H, alkyl, aryl, heterocyclyl or lower alkoxy and Rγ is H, lower alkyl, or Rγ and R₉ combined are alkylene which together with the carbon atom to which they are attached form a 3- to 7-membered ring; or Rγ′ and X combined are alkylene which together with the carbon atom to which they are attached form a 3- to 7-membered ring or
Z is —NRτ-C(O)—NH—S(O)₂—, wherein Rτ is H or lower alkyl,
X is hydrogen, hydroxy, NH₂, halogen, alkoxy, alkylthio, alkyl, —S(O)—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
R₁ is hydrogen, —C(O)R₄, —C(O)NR₅R₆ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂ and R₃ are, independently from each other, hydrogen, halogen, (C₁₋₃)alkyl or (C₁₋₃)alkoxy;
or a pharmaceutically acceptable salt thereof,
and wherein n+m+p is >1 or is 0, when X is aryl, and Y and Z are absent,
n+m+p is not 0 when X is —O-aryl, and Y and Z are absent, or
n+m+p is not 0 when X is —S-aryl, and Y and Z are absent, or
n+m+p is not 0 when X is —CH₂-aryl, and Y and Z are absent, or
n+m+p is not 0 when X is aryl, Z is absent and Y is —O— or Y is —S—, or
wherein Q cannot be —CH₂-aryl, —S-aryl or —O-aryl.

Preferably, the orientation of the Z function is with the X group on the right side of the listed function -Z→X e.g. Z is —NRα′-C(O)— means Z is —NRα′-C(O)—X.

Preferred are the compounds of formula (I), designated as the ALPHA group, wherein

Q is: —Y—(CH₂)ₙ—(CR₈R₉)ₚ—(CH₂)ₘ-Z-X in which
Y is oxygen or S(O)_{q} in which q is zero or an integer of 1 or 2; or

### Y is —C≡C— or —C═C—; or

Y is cyclopropyl or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
R₈ and R₉ combined are alkylene which together with the carbon atom to which they are attached form a 3- to 7-membered ring;
p is zero or an integer selected from 1 or 2
Z is absent;

### Z is —C(O)—O—; or

### Z is —C(O)—; or

Z is —C(O)—NRα-alkylene- or —C(O)—NRα-alkylene-O—, wherein Rα is H or lower alkyl; or
Z is —CO—NRα-(CH₂)_{n′}—(CR₈R_{9′})_{p′}—(CH₂)_{m′}—, or —C(O)—NRα-(CH₂)_{n′}—(CR_{8′}R_{9′})_{p′}—(CH₂)_{m′}—O—, wherein p′ is zero or an integer of 1, n′ and m′ are, independently from each other, zero or an integer from 1 to 8, R_{8′} and R_{9′} are, independently from each other, hydrogen or lower alkyl, Rα is H or lower alkyl; or
Z is —NRα′-C(O)—, or —NRα′-C(O)—O—, wherein Rα′ is H or lower alkyl, or Rα′ and R₉ combined are alkylene which together with the carbon atom to which they are attached form a 3- to 7-membered ring; or

### Z is —C(O)—NH—NH—C(O)—O—; or

### Z is —S(O)₂—, or —S(O)—; or

Z is —NRβ-S(O)₂—, wherein R₁₃ is H, lower alkyl, or Rβ and R₉ combined are alkylene which together with the carbon atom to which they are attached form a 3- to 7-membered ring; or

### Z is —NH—S(O)₂—NH—C(O)—O—; or

Z is —NRγ—C(O)—NRγ′; wherein Rγ′ is H, alkyl, aryl, heterocyclyl, or lower alkoxy and Rγ is H, lower alkyl, or Rγ and R₉ combined are alkylene which together with the carbon atom to which they are attached form a 3- to 7-membered ring; or Rγ′ and X combined are alkylene which together with the carbon atom to which they are attached form a 3- to 7-membered ring or
Z is NRτ-C(O)—NH—S(O)₂—, wherein Rτ is H or lower alkyl,
X is hydrogen, hydroxy, NH₂, halogen, alkoxy, alkylthio, alkyl, —S(O)—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
R₁ is hydrogen, —C(O)R₄, —C(O)NR₅R₆ or —C(O)OR₇ in which R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂ and R₃ are, independently from each other, hydrogen, halogen, (C₁₋₃)alkyl or (C₁₋₃)alkoxy;
or a pharmaceutically acceptable salt thereof,
and wherein n+m+p is >1 or is 0, when X is aryl, and Y and Z are absent,
n+m+p is not 0 when X is —O-aryl, and Y and Z are absent, or
n+m+p is not 0 when X is —S-aryl, and Y and Z are absent, or
n+m+p is not 0 when X is —CH₂-aryl, and Y and Z are absent, or
n+m+p is not 0 when X is aryl, Z is absent and Y is —O— or Y is —S—, or
wherein Q cannot be —CH₂-aryl, —S-aryl or —O-aryl.

Preferably, the orientation of the Z function is with the X group on the right side of the listed function -Z→X e.g. Z is —NRα′-C(O)— means Z is —NRα′-C(O)—X.

Preferred are the compounds in the ALPHA group wherein;

Y is oxygen; or

### Y is —C≡C— or —C═C—; or

Y is cyclopropyl or
Y is absent; and
X is, hydrogen, hydroxy, NH₂, halogen, alkoxy, alkylthio, alkyl, —S(O)—OH, alkyl, cycloalkyl, cyano, trifluoromethyl, free or esterified carboxy, heterocyclyl, heteroaryl, heteroaralkyl, aryl, aralkyl, aryloxy;

Preferred are the compounds in the ALPHA group wherein

R₂ and R₃ are hydrogen;
or a pharmaceutically acceptable salt thereof.

Preferred are the compounds in the ALPHA group wherein

R₁ is hydrogen;
or a pharmaceutically acceptable salt thereof.

Further preferred are the compounds in the ALPHA group wherein

n is zero or an integer from 1 to 4;
m is zero or an integer from 1 to 4;
p is zero or 1;
or a pharmaceutically acceptable salt thereof.

Especially preferred are the compounds in the ALPHA group, wherein

m+n+p is between 0 and 7 or preferably between 0 and 5,
or a pharmaceutically acceptable salt thereof.

Preferred are the compounds of formula (I), designated as the A group, wherein

Q is —Y—(CH₂)ₙ—(CR₈R₉)ₚ—(CH₂)ₘ-Z-X, in which
Y is oxygen or S(O)_{q} in which q is zero or an integer of 1 or 2; or

### Y is —C≡C— or —C═C—; or

Y is cyclopropyl; or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl;
p is zero or an integer selected from 1 or 2
Z is absent;

### Z is —CO—O—; or

### Z is —CO—; or

X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
or a pharmaceutically acceptable salt thereof.

Preferred are the compounds in the A group wherein

Y is oxygen; or
Y is cyclopropyl; or
Y is absent;
or a pharmaceutically acceptable salt thereof.

Preferred are the compounds in the A group wherein

R₈ and R₉ are, independently from each other, hydrogen, alkoxy, alkanoyl, alkoxycarbonyl, aralkyl, aryl, or alkyl;
or a pharmaceutically acceptable salt thereof.

Preferred are the compounds in the A group wherein

X is hydrogen, hydroxy, alkyl, heterocyclyl, heteroaryl, aryl;
or a pharmaceutically acceptable salt thereof.

Preferred are the compounds in the A group wherein

R₂ and R₃ are hydrogen;
or a pharmaceutically acceptable salt thereof.

Preferred are the compounds in the A group wherein

R₁ is hydrogen;
or a pharmaceutically acceptable salt thereof.

Further preferred are the compounds in the A group wherein

n is zero or an integer from 1 to 3;
m is zero or an integer from 1 to 3;
p is zero or 1;
or a pharmaceutically acceptable salt thereof.

Especially preferred are the compounds in the A group, wherein

m+n+p is between 0 and 4,
or a pharmaceutically acceptable salt thereof.

Other preferred compounds are the compounds in the A group, wherein

m+n+p is between 1 and 3, and
n is 1,
or a pharmaceutically acceptable salt thereof.

Other preferred compounds are the compounds in the A group, wherein

X is phenyl.

Preferred are the compounds of formula (I), designated as B, wherein;

Q is —Y—(CH₂)ₙ—(CR₈R₉)ₚ—(CH₂)ₘ-Z-X, in which
Y is oxygen or S(O)_{q} in which q is zero or an integer of 1 or 2; or

### Y is —C≡C— or —C═C—; or

Y is cyclopropyl; or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
R₈ and R₉ combined are alkylene which together with the carbon atom to which they are attached form a 3- to 7-membered ring;
p is zero or an integer selected from 1 or 2
Z is absent;
X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
or a pharmaceutically acceptable salt thereof.

Preferred are the compounds in the B group wherein

R₈ and R₉ are, independently from each other, hydrogen, alkoxy, alkanoyl, alkoxycarbonyl, aralkyl, or alkyl;
or a pharmaceutically acceptable salt thereof.

Preferred are the compounds in the B group wherein

X is hydrogen, NH₂, hydroxy, alkylthio, —SO—OH, alkyl, cycloalkyl, cyano, trifluoromethyl, free or esterified carboxy, heterocyclyl, heteroaryl, aryl;
or a pharmaceutically acceptable salt thereof.

Preferred are the compounds in the B group wherein

R₂ and R₃ are hydrogen;
or a pharmaceutically acceptable salt thereof.

Preferred are the compounds in the B group wherein

R₁ is hydrogen;
or a pharmaceutically acceptable salt thereof.

Further preferred are the compounds in the B group wherein

n is zero or an integer from 1 to 3;
m is zero or an integer from 1 to 3;
p is zero or 1;
or a pharmaceutically acceptable salt thereof.

Especially preferred are the compounds in the B group, wherein

m+n+p is between 0 and 6 or preferably 0 and 4,
or a pharmaceutically acceptable salt thereof.

Other preferred compounds are the compounds in the B group, wherein

m+n is between 0 and 6 or preferably 0 and 4, and
p is 0,
or a pharmaceutically acceptable salt thereof.

Other preferred compounds are the compounds in the B group, wherein

X is selected from phenyl or heteroaryl, preferably unsubstituted or substituted by at least one substituent e.g. one or two, which is preferably a substituent selected from carboxy, carbamoyl, and lower alkyl,
or a pharmaceutically acceptable salt thereof.

Other preferred compounds are the compounds in the B group, wherein

m+n is 1, 2 or 3, preferably 1 or 2,
m+m+p is preferably 2 or 3,
p is 1 or 0, and
X is cycloalkyl, heterocyclyl, heteroaryl, or aryl, preferably unsubstituted or substituted by at least one substituent e.g. one or two, which is preferably a substituent selected from sulfonamido, carboxy, carbamoyl, and lower alkyl,
or a pharmaceutically acceptable salt thereof.

Other preferred compounds are the compounds in the B group, wherein

m+n is 1, 2 or 3, preferably 1 or 2,
m+n+p is 2, 3 or 4, preferably 2 or 3,
p is 1 or 0, and
X is aryl, preferably unsubstituted or substituted by at least one substituent e.g. one or two, which is preferably a substituent selected from sulfonamido, carboxy, carbamoyl, and lower alkyl,
or a pharmaceutically acceptable salt thereof.

Other preferred compounds are the compounds in the B group, wherein

m+n is 1, 2 or 3, preferably 1 or 2,
p is 1 or 0, and
X is “amide” type heterocyclyl, cycloalkyl substituted by at least one substituent e.g. one or two, which is preferably sulfonamide, or aryl substituted by at least one substituent e.g. one or two, which is preferably sulfonamido
or a pharmaceutically acceptable salt thereof.

Preferred are the compounds of formula (I), designated as the C, wherein

Q is —Y—(CH₂)ₙ—(CR₈R₉)ₚ—(CF₁₂)ₘ-Z-X, in which
Y is oxygen or S(O)_{q} in which q is zero or an integer of 1 or 2; or

### Y is —C≡C— or —C═C—; or

Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
R₈ and R₉ combined are alkylene which together with the carbon atom to which they are attached form a 3- to 7-membered ring;
p is zero or an integer selected from 1 or 2
Z is —CO—NRα-alkylene- or —CO—NRα-alkylene-O—, wherein Rα is H or lower alkyl; or
Z is —CO—NRα-(CH₂)_{n′}—(CR_{8′}R_{9′})_{p′}—(CH₂)_{m′}—, or
—CO—NRα-(CH₂)_{n′}—(CR_{8′}R_{9′})_{p′}—(CH₂)_{m′}—O—,
wherein p′ is zero or an integer of 1, n′ and m′ are independently from each other, zero or an integer from 1 to 8, R_{8′} and R_{9′} are, independently from each other, hydrogen or lower alkyl, Rα is H or lower alkyl; or
Z is —NRα′-CO—, or —NRα′-CO—O—, wherein Rα′ is, H or lower alkyl, or Rα′ and R₉ combined are alkylene which together with the carbon atom to which they are attached form a 3- to 7-membered ring; or

### Z is —CO—NH—NH—CO—O—; or

X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
or a pharmaceutically acceptable salt thereof.

Preferred are the compounds in the C group wherein

Y is absent;
or a pharmaceutically acceptable salt thereof.

Preferred are the compounds in the C group wherein

R₈ and R₉ are, independently from each other, hydrogen, alkanoylamino, aralkyl, aryl, or alkyl;
or a pharmaceutically acceptable salt thereof.

Preferred are the compounds in the C group wherein

X is hydrogen, alkyl, cycloalkyl, free or esterified carboxy, aryl, aralkyl, aryloxy;
or a pharmaceutically acceptable salt thereof.

Preferred are the compounds in the C group wherein

R₂ and R₃ are hydrogen;
or a pharmaceutically acceptable salt thereof.

Preferred are the compounds in the C group wherein

R₁ is hydrogen;
or a pharmaceutically acceptable salt thereof.

Further preferred are the compounds in the C group wherein

n is zero or an integer from 1 to 3;
m is zero or an integer from 1 to 3;
p is zero or 1;
or a pharmaceutically acceptable salt thereof.

Especially preferred are the compounds in the C group, wherein

m+n+p is between 0 and 6 or preferably between 0 and 4,
or a pharmaceutically acceptable salt thereof.

Other preferred compounds are the compounds in the C group, wherein

m+n+p is between 1 and 3 (i.e. 1, 2 or 3)
m+n is between 1 and 3 (i.e. 1, 2 or 3) and p is 0
m+n+p is between 1 and 3 (i.e. 1, 2 or 3) and p is 1
m is 0, n is between 1 and 2 (i.e. 1, or 2) and p is 1
or a pharmaceutically acceptable salt thereof.

Especially preferred are the compounds in the C group, wherein

n′ and m′ are independently from each other, zero or an integer from 1 to 6, and
p′ is zero or an integer of 1, or a pharmaceutically acceptable salt thereof.

Especially preferred are the compounds in the C group, wherein

p′+n′+m′ is comprised between zero and 5, or between 3 and 5 i.e. 3, 4 or 5, or a pharmaceutically acceptable salt thereof.

Especially preferred are the compounds in the C group, wherein

n′ and m′ are independently from each other, zero or an integer from 1 to 6, preferably from 1 to 4,
or a pharmaceutically acceptable salt thereof.

Other preferred compounds are the compounds in the C group, wherein

n′+m′ is between 0 and 5, or between 3 and 5, preferably 4, and
p′ is 0,
or a pharmaceutically acceptable salt thereof.

Especially preferred are the compounds in the C group, wherein

X is phenyl, preferably unsubstituted or substituted preferably by at least one, e.g. one or two, of the substituents selected preferably from alkoxycarbonyl, carboxy, alkoxy, cyano, lower alkyl, (lower alkyl)-NHC(O)—, (lower alkyl)₂-NC(O)— and hydroxy.

Preferred are the compounds of formula (I), designated as the D group, wherein

Q is —Y—(CH₂)ₙ—(CR₈R₉)ₚ—(CH₂)ₘ-Z-X, in which
Y is absent;
n and m are, independently from each other, zero;
p is zero;
Z is absent;
X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
or a pharmaceutically acceptable salt thereof.

Preferred are the compounds in the D group wherein

X is halogen, cyano, trifluoromethyl, heterocyclyl, heteroaryl, aryl,
or a pharmaceutically acceptable salt thereof.

Preferred are the compounds in the D group wherein

R₂ and R₃ are hydrogen;
or a pharmaceutically acceptable salt thereof.

Preferred are the compounds in the D group wherein

R₁ is hydrogen;
or a pharmaceutically acceptable salt thereof.

Further preferred are the compounds in the D group wherein

X is aryl or heteroaryl, or a pharmaceutically acceptable salt thereof.

Especially preferred are the compounds in the D group, wherein

X is aryl substituted by an “amide” type heterocyclyl, or a pharmaceutically acceptable salt thereof.

Preferred are the compounds of formula (I), designated as the E group, wherein

Q is —Y—(CH₂)ₙ—(CR₈R₉)ₚ—(CH₂)ₘ-Z-X, in which
Y is oxygen or S(O)_{q} in which q is zero or an integer of 1 or 2; or

### Y is —C≡C— or —C═C—; or

Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
R₈ and R₉ combined are alkylene which together with the carbon atom to which they are attached form a 3- to 7-membered ring;
p is zero or an integer selected from 1 or 2

### Z is —SO₂—, or —SO—; or

Z is —NRβ-SO₂—, wherein Rβ is H, lower alkyl, or Rβ and R₉ combined are alkylene which together with the carbon atom to which they are attached form a 3- to 7-membered ring preferably 5-, 6- or 7-membered ring; or

### Z is —NH—SO₂—NH—CO—O—; or

X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
or a pharmaceutically acceptable salt thereof.

Preferred are the compounds in the E group wherein

Y is absent; or a pharmaceutically acceptable salt thereof.

Preferred are the compounds in the E group wherein

R₈ and R₉ are, independently from each other, hydrogen, aralkyl, heteroaryl, heterocyclyl, heterocyclyl, carbamoyl; or
R₈ and R₉ combined are alkylene which together with the carbon atom to which they are attached form a 3- to 7-membered ring;
or a pharmaceutically acceptable salt thereof.

Preferred are the compounds in the E group wherein

X is hydrogen, alkyl, cycloalkyl, heteroaryl, aryl, aralkyl;
or a pharmaceutically acceptable salt thereof.

Preferred are the compounds in the E group wherein

R₂ and R₃ are hydrogen;
or a pharmaceutically acceptable salt thereof.

Preferred are the compounds in the E group wherein

R₁ is hydrogen;
or a pharmaceutically acceptable salt thereof.

Further preferred are the compounds in the E group wherein

n is zero or an integer from 1 to 4;
m is zero or an integer from 1 to 4;
p is zero or 1;
or a pharmaceutically acceptable salt thereof.

Especially preferred are the compounds in the E group, wherein

m+n+p is between 0 and 7 or preferably between 0 and 5,
or a pharmaceutically acceptable salt thereof.

Other preferred compounds are the compounds in the E group, wherein

i) m+n+p is 2 or 3, or
ii) m+n is 2 or 3, and p is 0, or
iii) n is 1 or 2, m is 0 or 1, and p is 1 when Rβ and R₉ combined are alkylene which together with the carbon atom to which they are attached form a 5-, 6- or 7-membered ring
or a pharmaceutically acceptable salt thereof.

Other preferred compounds are the compounds in the E group, wherein

m+n is 1 or 2, m is 0 or 1, and p is 1, or
n is 1 or 2, m is 0 or 1, and p is 1 when R₈ is hydrogen and R₉ is selected from aralkyl, heteroaryl, heterocyclyl, heterocyclyl, or carbamoyl;
or a pharmaceutically acceptable salt thereof.

Other preferred compounds are the compounds in the E group, wherein

X is selected from phenyl, biphenyl, benzyl, lower alkyl, methyl substituted by on or two phenyl, ethyl substituted by one or two pheny, or methyl substituted by cycloalkyl

Preferred are the compounds of formula (I), designated as the F group, wherein

Q is —Y—(CH₂)ₙ—(CR₈R₉)ₚ—(CH₂)ₘ-Z-X, in which
Y is oxygen or S(O)_{q} in which q is zero or an integer of 1 or 2; or

### Y is —C≡C— or —C═C—; or

Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
R₈ and R₉ combined are alkylene which together with the carbon atom to which they are attached form a 3- to 7-membered ring;
p is zero or an integer selected from 1 or 2
Z is —NRγ-CO—NRγ′—; wherein Rγ′ is H, alkyl, aryl, heterocyclyl, or lower alkoxy and Rγ is H, lower alkyl, or Rγ and R₉ combined are alkylene which together with the carbon atom to which they are attached form a 3- to 7-membered ring; or Rγ′ and X combined are alkylene which together with the carbon atom to which they are attached form a 3- to 7-membered ring; or
Z is —NRτ-CO—NH—SO₂—, wherein Rτ is H or lower alkyl,
X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
or a pharmaceutically acceptable salt thereof.

Preferred are the compounds in the F group wherein

Y is absent; or a pharmaceutically acceptable salt thereof.

Preferred are the compounds in the F group wherein

R₈ and R₉ are, independently from each other, hydrogen;
or a pharmaceutically acceptable salt thereof.

Preferred are the compounds in the F group wherein

X is hydrogen, alkyl, cycloalkyl, heterocyclyl, aryl, aralkyl;
or a pharmaceutically acceptable salt thereof.

Preferred are the compounds in the F group wherein

R₂ and R₃ are hydrogen;
or a pharmaceutically acceptable salt thereof.

Preferred are the compounds in the F group wherein

Rγ′ is H or lower alkyl, or a pharmaceutically acceptable salt thereof.

Preferred are the compounds in the F group wherein

R₁ is hydrogen;
or a pharmaceutically acceptable salt thereof.

Especially preferred are the compounds in the F group, wherein

m+n+p is between 0 and 7 or preferably between 0 and 5 or between 2 and 3,
or a pharmaceutically acceptable salt thereof.

Further preferred are the compounds in the F group wherein

n is zero or an integer from 1 to 4;
m is zero or an integer from 1 to 4;
p is zero or 1;
or a pharmaceutically acceptable salt thereof.

Especially preferred are the compounds in the F group, wherein

m+n+p is 2 or 3, and
X is lower alkyl, phenyl, benzyl, or cyclohexyl,
or a pharmaceutically acceptable salt thereof.

Compound according to any of the above described groups wherein;

the term alkyl preferably refers to a lower alkyl,
aryl is preferably a phenyl, and/or
when R₈ and R₉ are present, at least one of R₈ or R₉ is hydrogen.

Particular embodiments of the compounds are: the below specific exemplified compounds,
3-{2-[3-Hydroxy-4-(1,1,4-trioxo-1,2,5-thiadiazolidin-2-yl)-phenyl]-ethyl}-benzamide
3-{2-[3-Hydroxy-4-(1,1,4-trioxo-1,2,5-thiadiazolidin-2-yl)-phenyl]-ethyl}-N-methyl benzamide
3-{2-[3-Hydroxy-4-(1,1,4-trioxo-1,2,5-thiadiazolidin-2-yl)-phenyl]-ethyl}-N,N-dimethylbenzamide
4-{2-[3-Hydroxy-4-(1,1,4-trioxo-1,2,5-thiadiazolidin-2-yl)-phenyl]-ethyl}-N,N-dimethylbenzamide
4-{2-[3-Hydroxy-4-(1,1,4-trioxo-1,2,5-thiadiazolidin-2-yl)-phenyl]-ethyl}-benzamide
4-{2-[3-Hydroxy-4-(1,1,4-trioxo-1,2,5-thiadiazolidin-2-yl)-phenyl]-ethyl}-N-methylbenzamide
3-{2-[3-Hydroxy-4-(1,1,4-trioxo-1,2,5-thiadiazolidin-2-yl)-phenyl]-ethyl}-benzoic acid
4-{2-[3-Hydroxy-4-(1,1,4-trioxo-1,2,5-thiadiazolidin-2-yl)-phenyl]-ethyl}-benzoic acid
4-{2-[3-Hydroxy-4-(1,1,4-trioxo-1,2,5-thiadiazolidin-2-yl)-phenyl]-ethyl}-benzonitrile
2-{2-[3-Hydroxy-4-(1,1,4-trioxo-1,2,5-thiadiazolidin-2-yl)-phenyl]-ethyl}-benzonitrile
5-(2-Hydroxy-4-phenethylphenyl)-1,1-dioxo-1,2,5-thiadiazolidin-3-one
5-{2-Hydroxy-4-[2-(3-methoxyphenyl)-ethyl]-phenyl}-1,1-dioxo-1,2,5-thiadiazolidin-3-one
5-{-4-[2-(3-Fluorophenyl)-ethyl]-2-hydroxyphenyl}-1,1-dioxo-1,2,5-thiadiazolidin-3-one
5-{-4-[2-(2-Fluorophenyl)-ethyl]-2-hydroxyphenyl}-1,1-dioxo-1,2,5-thiadiazolidin-3-one
5-[2-Hydroxy-4-(2-pentafluorophenylethyl)-phenyl]-1,1-dioxo-1,2,5-thiadiazolidin-3-one
5-[2-Hydroxy-4-(2-p-tolylethyl)-phenyl]-1,1-dioxo-1,2,5-thiadiazolidin-3-one
5-{2-Hydroxy-4-[2-(4-octylphenyl)-ethyl]-phenyl}-1,1-dioxo-1,2,5-thiadiazolidin-3-one
5-[4-(2-Biphenyl-4-yl-ethyl)-2-hydroxyphenyl]-1,1-dioxo-1,2,5-thiadiazolidin-3-one
5-{4-[2-(4-tert-Butylphenyl)-ethyl]-2-hydroxyphenyl}-1,1-dioxo-1,2,5-thiadiazolidin-3-one
5-{4-[2-(2,5-Dimethylphenyl)-ethyl]-2-hydroxyphenyl}-1,1-dioxo-1,2,5-thiadiazolidin-3-one
5-{4-[2-(2,4-Dimethylphenyl)-ethyl]-2-hydroxyphenyl}-1,1-dioxo-1,2,5-thiadiazolidin-3-one
5-{2-Hydroxy-4-[2-(4-trifluoromethylphenyl)-ethyl]-phenyl}-1,1-dioxo-1,2,5-thiadiazolidin-3-one
Acetic acid 4-{2-[3-hydroxy-4-(1,1,4-trioxo-1,2,5-thiadiazolidin-2-yl)-phenyl]-ethyl}-phenyl ester
5-{2-Hydroxy-4-[2-(4-phenoxyphenyl)-ethyl]-phenyl}-1,1-dioxo-1,2,5-thiadiazolidin-3-one
5-[2-Hydroxy-4-(2-pyridin-4-ylethyl)-phenyl]-1,1-dioxo-1,2,5-thiadiazolidin-3-one
5-[2-Hydroxy-4-(2-pyridin-3-yl-ethyl)-phenyl]-1,1-dioxo-1,2,5-thiadiazolidin-3-one
5-[2-Hydroxy-4-(2-naphthalenethyl)-phenyl]-1,1-dioxo-1,2,5-thiadiazolidin-3-one
5-[2-Hydroxy-4-(2-quinolin-3-yl-ethyl)-phenyl]-1,1-dioxo-1,2,5-thiadiazolidin-3-one
5-{4-[2-(4,6-Diamino-[1,3,5]triazin-2-yl)-ethyl]-2-hydroxy-phenyl}-1,1-dioxo-1,2,5-thiadiazolidin-3-one
5-[2-Hydroxy-4-(2-phenylpropyl)-phenyl]-1,1-dioxo-1,2,5-thiadiazolidin-3-one
5-{4-[2-(2-Aminophenyl)-propyl]-2-hydroxyphenyl}-1,1-dioxo-1,2,5-thiadiazolidin-3-one
3-[3-Hydroxy-4-(1,1,4-trioxo-1,2,5-thiadiazolidin-2-yl)-phenyl]-2-phenylpropionic acid ethyl ester
5-[2-Hydroxy-4-(1-methyl-2-phenylethyl)-phenyl]-1,1-dioxo-1,2,5-thiadiazolidin-3-one
5-{2-Hydroxy-4-[2-(6-methoxypyridin-2-yl)-ethyl]-phenyl}-1,1-dioxo-1,2,5-thiadiazolidin-3-one
5-[2-Hydroxy-4-((E)-2-pyridin-3-yl-vinyl)-phenyl]-1,1-dioxo-1,2,5-thiadiazolidin-3-one
5-[2-Hydroxy-4-(1-methoxy-2-phenylethyl)-phenyl]-1,1-dioxo-1,2,5-thiadiazolidin-3-one
5-[2-Hydroxy-4-(3-oxo-2-phenylbutyl)-phenyl]-1,1-dioxo-1,2,5-thiadiazolidin-3-one
5-{2-Hydroxy-4-[2-(2H-pyrazol-3-yl)-ethyl]-phenyl}-1,1-dioxo-1,2,5-thiadiazolidin-3-one
5-{2-Hydroxy-4-[2-(1H-pyrazol-4-yl)-ethyl]-phenyl}-1,1-dioxo-1,2,5-thiadiazolidin-3-one
5-{2-Hydroxy-4-[2-(1-methyl-1H-pyrazol-4-yl)-ethyl]-phenyl}-1,1-dioxo-1,2,5-thiadiazolidin-3-one
5-[2-Hydroxy-4-(2-thiazol-5-yl-ethyl)-phenyl]-1,1-dioxo-1,2,5-thiadiazolidin-3-one
5-{4-[2-(2,4-Dimethyl-thiazol-5-yl)-ethyl]-2-hydroxyphenyl}-1,1-dioxo-1,2,5-thiadiazolidin-3-one
5-[2-Hydroxy-4-(2-[1,2,4]triazol-yl-ethyl)-phenyl]-1,1-dioxo-1,2,5-thiadiazolidin-3-one
5-[2-Hydroxy-4-(2-imidazol-1-yl-ethyl)-phenyl]-1,1-dioxo-1,2,5-thiadiazolidin-3-one
5-{2-Hydroxy-4-[2-(2-methyl-thiazol-5-yl)-ethyl]-phenyl}-1,1-dioxo-1,2,5-thiadiazolidin-3-one
5-{2-Hydroxy-4-[2-(2-propyl-thiazol-5-yl)-ethyl]-phenyl}-1,1-dioxo-1,2,5-thiadiazolidin-3-one
5-(2-Hydroxy-4-{2-[4-methyl-2-(4-trifluoromethyl-phenyl)-thiazol-5-yl]-ethyl}-phenyl)-1,1-dioxo-1,2,5-thiadiazolidin-3-one
5-{2-Hydroxy-4-[2-(2-methyl-4-trifluoromethyl-thiazol-5-yl)-ethyl]-phenyl}-1,1-dioxo-1,2,5-thiadiazolidin-3-one
5-{4-[2-(1H-Benzoimidazol-2-yl)-ethyl]-2-hydroxyphenyl}-1,1-dioxo-1,2,5-thiadiazolidin-3-one
5-[2-Hydroxy-4-(3-phenylpropyl)-phenyl]-1,1-dioxo-1,2,5-thiadiazolidin-3-one
5-{4-[3-(3,4-Dimethoxyphenyl)-propyl]-2-hydroxyphenyl}-1,1-dioxo-1,2,5-thiadiazolidin-3-one
5-[2-Hydroxy-4-(2-methyl-3-phenylpropyl)-phenyl]-1,1-dioxo-1,2,5-thiadiazolidin-3-one
5-[2-Hydroxy-4-(3-hydroxy-3-phenylpropyl)-phenyl]-1,1-dioxo-1,2,5-thiadiazolidin-3-one
5-(2-Hydroxy-4-phenethyloxyphenyl)-1,1-dioxo-1,2,5-thiadiazolidin-3-one
5-[2-Hydroxy-4-(4-phenylbutyl)-phenyl]-1,1-dioxo-1,2,5-thiadiazolidin-3-one
{3-[3-Hydroxy-4-(1,1,4-trioxo-1,2,5-thiadiazolidin-2-yl)-phenyl]-propyl}-carbamic acid tert-butyl ester
5-[4-(3-Aminopropyl)-2-hydroxyphenyl]-1,1-dioxo-1,2,5-thiadiazolidin-3-one
{2-[3-Hydroxy-4-(1,1,4-trioxo-1,2,5-thiadiazolidin-2-yl)-phenyl]-ethyl}-carbamic acid tert-butyl ester
{(S)-1-Benzyl-2-[3-hydroxy-4-(1,1,4-trioxo-1,2,5-thiadiazolidin-2-yl)-phenyl]-ethyl}-carbamic acid tert-butyl ester
{3-[3-Hydroxy-4-(1,1,4-trioxo-1,2,5-thiadiazolidin-2-yl)-phenyl]-1,1-dimethylpropyl}-carbamic acid tert-butyl ester
2-{2-[3-Hydroxy-4-(1,1,4-trioxo-1,2,5-thiadiazolidin-2-yl)-phenyl]-ethyl}-piperidine-1-carboxylic acid tert-butyl ester
2-{2-[3-Hydroxy-4-(1,1,4-trioxo-1,2,5-thiadiazolidin-2-yl)-phenyl]-ethyl}-azepane-1-carboxylic acid tert-butyl ester
3-[3-Hydroxy-4-(1,1,4-trioxo-1,2,5-thiadiazolidin-2-yl)-benzyl]-piperidine-1-carboxylic acid tert-butyl ester
5-(2-Hydroxy-4-piperidin-3-ylmethylphenyl)-1,1-dioxo-1,2,5-thiadiazolidin-3-one
{(1R*,2S*)-2-[3-Hydroxy-4-(1,1,4-trioxo-1,2,5-thiadiazolidin-2-yl)-benzyl]-cyclohexyl}-carbamic acid tert-butyl ester
N-{2-[3-Hydroxy-4-(1,1,4-trioxo-1,2,5-thiadiazolidin-2-yl)-phenyl]-ethyl}-benzamide
4-Fluoro-N-{2-[3-hydroxy-4-(1,1,4-trioxo-1,2,5-thiadiazolidin-2-yl)-phenyl]-ethyl}-benzamide
N-{2-[3-Hydroxy-4-(1,1,4-trioxo-1,2,5-thiadiazolidin-2-yl)-phenyl]-ethyl}-acetamide
N-{2-[3-Hydroxy-4-(1,1,4-trioxo-1,2,5-thiadiazolidin-2-yl)-phenyl]-ethyl}-propionamide
N-{2-[3-Hydroxy-4-(1,1,4-trioxo-1,2,5-thiadiazolidin-2-yl)-phenyl]-ethyl}-isobutyramide
N-{2-[3-Hydroxy-4-(1,1,4-trioxo-1,2,5-thiadiazolidin-2-yl)-phenyl]-ethyl}-2,2-dimethyl-propionamide
Adamantane-1-carboxylic acid {2-[3-hydroxy-4-(1,1,4-trioxo-1,2,5-thiadiazolidin-2-yl)-phenyl]-ethyl}-amide
N-{3-[3-Hydroxy-4-(1,1,4-trioxo-1,2,5-thiadiazolidin-2-yl)-phenyl]-propyl}-acetamide
4-Fluoro-N-{3-[3-hydroxy-4-(1,1,4-trioxo-1,2,5-thiadiazolidin-2-yl)-phenyl]-propyl}-benzamide
-{3-[3-Hydroxy-4-(1,1,4-trioxo-1,2,5-thiadiazolidin-2-yl)-phenyl]-propyl}-propionamide
N-{3-[3-Hydroxy-4-(1,1,4-trioxo-1,2,5-thiadiazolidin-2-yl)-phenyl]-propyl}-isobutyramide
N-{3-[3-Hydroxy-4-(1,1,4-trioxo-1,2,5-thiadiazolidin-2-yl)-phenyl]-propyl}-2,2-dimethyl-propionamide
Adamantane-1-carboxylic acid {3-[3-hydroxy-4-(1,1,4-trioxo-1,2,5-thiadiazolidin-2-yl)-phenyl]-propyl}-amide
5-[2-Hydroxy-4-((S)-5-oxopyrrolidin-2-ylmethyl)-phenyl]-1,1-dioxo-1,2,5-thiadiazolidin-3-one
6-[3-Hydroxy-4-(1,1,4-trioxo-1,2,5-thiadiazolidin-2-yl)-benzyl]-1H-pyridin-2-one
6-[3-Hydroxy-4-(1,1,4-trioxo-1,2,5-thiadiazolidin-2-yl)-benzyl]-piperidin-2-one
7-[3-Hydroxy-4-(1,1,4-trioxo-1,2,5-thiadiazolidin-2-yl)-benzyl]-azepan-2-one
(R)-3-[3-Hydroxy-4-(1,1,4-trioxo-1,2,5-thiadiazolidin-2-yl)-benzyl]-3,4-dihydro-2H-isoquinolin-1-one
(S)-3-[3-Hydroxy-4-(1,1,4-trioxo-1,2,5-thiadiazolidin-2-yl)-benzyl]-2,3-dihydro-benzo[c]azepin-1-one
(R)-3-[3-Hydroxy-4-(1,1,4-trioxo-1,2,5-thiadiazolidin-2-yl)-benzyl]-2,3,4,5-tetrahydrobenzo[c]azepin-1-one
1-[3-Hydroxy-4-(1,1,4-trioxo-1,2,5-thiadiazolidin-2-yl)-benzyl]-1,2,4,5-tetrahydrobenzo[c]azepin-3-one
1-[3-Hydroxy-4-(1,1,4-trioxo-1,2,5-thiadiazolidin-2-yl)-benzyl]-1,3,4,5-tetrahydrobenzo[d]azepin-2-one
7-[3-Hydroxy-4-(1,1,4-trioxo-1,2,5-thiadiazolidin-2-yl)-benzyl]-6,7-dihydro-dibenzo[c,e]azepin-5-one
(S)-7-[3-Hydroxy-4-(1,1,4-trioxo-1,2,5-thiadiazolidin-2-yl)-benzyl]-6,7-dihydro-dibenzo[c,e]azepin-5-one
3-[3-Hydroxy-4-(1,1,4-trioxo-1,2,5-thiadiazolidin-2-yl)-benzyl]-3,4-dihydro-2H-naphtho[1,8-cd]azepin-1-one
5-{4-[2-(1-Acetylpiperidin-2-yl)-ethyl]-2-hydroxyphenyl}-1,1-dioxo-1,2,5-thiadiazolidin-3-one
N-{(1R*,2S*)-2-[3-Hydroxy-4-(1,1,4-trioxo-1,2,5-thiadiazolidin-2-yl)-benzyl]-cyclohexyl}-acetamide
N-{(S)-1-Benzyl-2-[3-hydroxy-4-(1,1,4-trioxo-1,2,5-thiadiazolidin-2-yl)-Phenyl]-ethyl}-2,2,2-trifluoroacetamide
N-{-4-[3-Hydroxy-4-(1,1,4-trioxo-1,2,5-thiadiazolidin-2-yl)-phenyl]-butyl}-Phthalamic acid
2-{4-[3-Hydroxy-4-(1,1,4-trioxo-1,2,5-thiadiazolidin-2-yl)-phenyl]-butyl}-isoindole-1,3-dione
3-[3-Hydroxy-4-(1,1,4-trioxo-1,2,5-thiadiazolidin-2-yl)-phenyl]-N-isopropyl-N-methylpropionamide
5-{4-[3-(3,4-Dihydro-1H-isoquinolin-2-yl)-3-oxopropyl]-2-hydroxyphenyl}-1,1-dioxo-1,2,5-thiadiazolidin-3-one
N′-{3-[3-Hydroxy-4-(1,1,4-trioxo-1,2,5-thiadiazolidin-2-yl)-phenyl]-propionyl}-hydrazinecarboxylic acid tert-butyl ester
N-Butyl-3-[3-hydroxy-4-(1,1,4-trioxo-1,2,5-thiadiazolidin-2-yl)-phenyl]-propionamide
3-[3-Hydroxy-4-(1,1,4-trioxo-1,2,5-thiadiazolidin-2-yl)-phenyl]-N-pentylpropionamide
N-Hexyl-3-[3-hydroxy-4-(1,1,4-trioxo-1,2,5-thiadiazolidin-2-yl)-phenyl]-propionamide
3-[3-Hydroxy-4-(1,1,4-trioxo-1,2,5-thiadiazolidin-2-yl)-phenyl]-N-(4-phenylbutyl)-propionamide
3-[3-Hydroxy-4-(1,1,4-trioxo-1,2,5-thiadiazolidin-2-yl)-phenyl]-N-(5-phenylpentyl)-propionamide
N-(2-Hydroxyphenyl)-3-[3-hydroxy-4-(1,1,4-trioxo-1,2,5-thiadiazolidin-2-yl)-phenyl]-propionamide
3-[3-Hydroxy-4-(1,1,4-trioxo-1,2,5-thiadiazolidin-2-yl)-phenyl]-N-phenylpropionamide
3-[3-Hydroxy-4-(1,1,4-trioxo-1,2,5-thiadiazolidin-2-yl)-phenyl]-N-o-tolyl-Propionamide
3-[3-Hydroxy-4-(1,1,4-trioxo-1,2,5-thiadiazolidin-2-yl)-phenyl]-N-isopropyl-propionamide
2-{3-[3-Hydroxy-4-(1,1,4-trioxo-1,2,5-thiadiazolidin-2-yl)-phenyl]-propionylamino}-2-methylpropionic acid
2-Hydroxy-6-(4-{3-[3-hydroxy-4-(1,1,4-trioxo-1,2,5-thiadiazolidin-2-yl)-phenyl]-propionylamino}-butoxy)-benzoic acid methyl ester
2-(4-{3-[3-Hydroxy-4-(1,1,4-trioxo-1,2,5-thiadiazolidin-2-yl)-phenyl]-propionylamino}-butoxy)-benzoic acid methyl ester
2-(4-{3-[3-Hydroxy-4-(1,1,4-trioxo-1,2,5-thiadiazolidin-2-yl)-phenyl]-propionylamino}-butoxy)-benzoic acid
3-[3-Hydroxy-4-(1,1,4-trioxo-1,2,5-thiadiazolidin-2-yl)-phenyl]-N-(4-phenoxybutyl)-propionamide
3-[3-Hydroxy-4-(1,1,4-trioxo-1,2,5-thiadiazolidin-2-yl)-phenyl]-N-[4-(2-trifluoromethylphenoxy)-butyl]-propionamide
3-[3-Hydroxy-4-(1,1,4-trioxo-1,2,5-thiadiazolidin-2-yl)-phenyl]-N-[4-(2-methanesulfonylphenoxy)-butyl]-propionamide
3-[3-Hydroxy-4-(1,1,4-trioxo-1,2,5-thiadiazolidin-2-yl)-phenyl]-N-[4-(3-methoxyphenoxy)-butyl]-propionamide
N-[4-(2,3-Dimethoxyphenoxy)-butyl]-3-[3-hydroxy-4-(1,1,4-trioxo-1,2,5-thiadiazolidin-2-yl)-phenyl]-propionamide
N-[4-(3-Hydroxyphenoxy)-butyl]-3-[3-hydroxy-4-(1,1,4-trioxo-1,2,5-thiadiazolidin-2-yl)-phenyl]-propionamide
N-[4-(2-Hydroxyphenoxy)-butyl]-3-[3-hydroxy-4-(1,1,4-trioxo-1,2,5-thiadiazolidin-2-yl)-phenyl]-propionamide
N-[4-(3-Hydroxy-2-methoxyphenoxy)-butyl]-3-[3-hydroxy-4-(1,1,4-trioxo-1,2,5-thiadiazolidin-2-yl)-phenyl]-propionamide
N-[4-(3-Hydroxy-2-methylphenoxy)-butyl]-3-[3-hydroxy-4-(1,1,4-trioxo-1,2,5-thiadiazolidin-2-yl)-phenyl]-propionamide
N-[4-(2-Acetyl-3-methoxyphenoxy)-butyl]-3-[3-hydroxy-4-(1,1,4-trioxo-1,2,5-thiadiazolidin-2-yl)-phenyl]-propionamide
2-Hydroxy-6-(4-{3-[3-hydroxy-4-(1,1,4-trioxo-1,2,5-thiadiazolidin-2-yl)-phenyl]-propionylamino}-butoxy)-N,N-dimethylbenzamide2-(4-{3-[3-Hydroxy-4-(1,1,4-trioxo-1,2,5-thiadiazolidin-2-yl)-phenyl]-propionylamino}-butoxy)-6, N,N-trimethyl benzamide
2-Fluoro-6-(4-{3-[3-hydroxy-4-(1,1,4-trioxo-1,2,5-thiadiazolidin-2-yl)-phenyl]-propionylamino}-butoxy)-N,N-dimethylbenzamide
2-Hydroxy-6-(4-{3-[3-hydroxy-4-(1,1,4-trioxo-1,2,5-thiadiazolidin-2-yl)-phenyl]-propionylamino}-butoxy)-benzoic acid
N-[4-(2-Acetyl-3-hydroxyphenoxy)-butyl]-3-[3-hydroxy-4-(1,1,4-trioxo-1,2,5-thiadiazolidin-2-yl)-phenyl]-propionamide
N-[4-(2-Cyano-3-hydroxyphenoxy)-butyl]-3-[3-hydroxy-4-(1,1,4-trioxo-1,2,5-thiadiazolidin-2-yl)-phenyl]-propionamide
N-[4-(3-Hydroxy-2-methanesulfinylphenoxy)-butyl]-3-[3-hydroxy-4-(1,1,4-trioxo-1,2,5-thiadiazolidin-2-yl)-phenyl]-propionamide
N-[4-(3-Hydroxy-2-methanesulfonylphenoxy)-butyl]-3-[3-hydroxy-4-(1,1,4-trioxo-1,2,5-thiadiazolidin-2-yl)-phenyl]-propionamide
2-(4-{2-Acetylamino-3-[3-hydroxy-4-(1,1,4-trioxo-1,2,5-thiadiazolidin-2-yl)-phenyl]-propionylamino}-butoxy)-6-hydroxybenzoic acid methyl ester
2-(4-{(S)-2-Acetylamino-3-[3-hydroxy-4-(1,1,4-trioxo-1,2,5-thiadiazolidin-2-yl)-phenyl]-propionylamino}-butoxy)-6-hydroxybenzoic acid methyl ester
3-[3-Hydroxy-4-(1,1,4-trioxo-1,2,5-thiadiazolidin-2-A-phenyl]-propionic acid methyl ester
3-[3-Hydroxy-4-(1,1,4-trioxo-1,2,5-thiadiazolidin-2-yl)-phenyl]-2-methylpropionic acid methyl ester
3-[3-Hydroxy-4-(1,1,4-trioxo-1,2,5-thiadiazolidin-2-yl)-phenyl]-2-methylpropionic acid tert-butyl ester
(1R*,2R*)-2-[3-Hydroxy-4-(1,1,4-trioxo-1,2,5-thiadiazolidin-2-yl)-phenyl]-cyclopropanecarboxylic acid ethyl ester
(1R*,2S*)-2-[3-Hydroxy-4-(1,1,4-trioxo-1,2,5-thiadiazolidin-2-yl)-phenyl]-cyclopropanecarboxylic acid ethyl ester
N-[3-Hydroxy-4-(1,1,4-trioxo-1,2,5-thiadiazolidin-2-yl)-benzyl]-N-methylbenzenesulfonamide
N-[3-Hydroxy-4-(1,1,4-trioxo-1,2,5-thiadiazolidin-2-yl)-benzyl]-N-methylmethanesulfonamide
C-Cyclohexyl-N-{2-[3-hydroxy-4-(1,1,4-trioxo-1,2,5-thiadiazolidin-2-yl)-phenyl]-ethyl}-methanesulfonamide
N-{2-[3-Hydroxy-4-(1,1,4-trioxo-1,2,5-thiadiazolidin-2-yl)-phenyl]-ethyl}-methansulfonamide
Ethanesulfonic acid {2-[3-hydroxy-4-(1,1,4-trioxo-1,2,5-thiadiazolidin-2-yl)-phenyl]ethyl}-amide
Butane-1-sulfonic acid {2-[3-hydroxy-4-(1,1,4-trioxo-1,2,5-thiadiazolidin-2-yl)-phenyl]-ethyl}-amide
Propane-2-sulfonic acid {2-[3-hydroxy-4-(1,1,4-trioxo-1,2,5-thiadiazolidin-2-yl)-phenyl]-ethyl}-amide
Octane-1-sulfonic acid {2-[3-hydroxy-4-(1,1,4-trioxo-1,2,5-thiadiazolidin-2-yl)-phenyl]-ethyl}-amide
N-{2-[3-Hydroxy-4-(1,1,4-trioxo-1,2,5-thiadiazolidin-2-yl)-phenyl]-ethyl}-benzenesulfonamide
N-{2-[3-Hydroxy-4-(1,1,4-trioxo-1,2,5-thiadiazolidin-2-yl)-phenyl]-ethyl}-C-phenyl-methansulfonamide
4-Fluoro-N-{2-[3-hydroxy-4-(1,1,4-trioxo-1,2,5-thiadiazolidin-2-yl)-phenyl]-ethyl}-benzenesulfonamide
3,4-Dichloro-N-{2-[3-hydroxy-4-(1,1,4-trioxo-1,2,5-thiadiazolidin-2-yl)-phenyl]-ethyl}-benzenesulfonamide
3-(4-{2-[3-Hydroxy-4-(1,1,4-trioxo-1,2,5-thiadiazoridin-2-yl)-phenyl]-ethylsulfamoyl}-phenyl)-propionic acid
2-Hydroxy-5-{2-[3-hydroxy-4-(1,1,4-trioxo-1,2,5-thiadiazolidin-2-yl)-phenyl]-ethylsulfamoyl}-benzoic acid
Naphthalene-1-sulfonic acid {2-[3-hydroxy-4-(1,1,4-trioxo-1,2,5-thiadiazolidin-2-yl)-phenyl]-ethyl}-amide
2-Naphthalen-1-yl-ethanesulfonic acid {2-[3-hydroxy-4-(1,1,4-trioxo-1,2,5-thiadiazolidin-2-yl)-phenyl]-ethyl}-amide
N-{3-[3-Hydroxy-4-(1,1,4-trioxo-1,2,5-thiadiazolidin-2-yl)-phenyl]-propyl}-methansulfonamide
N-{3-[3-Hydroxy-4-(1,1,4-trioxo-1,2,5-thiadiazolidin-2-yl)-phenyl]-propyl}-benzenesulfonamide
N-{3-[3-Hydroxy-4-(1,1,4-trioxo-1,2,5-thiadiazolidin-2-yl)-phenyl]-propyl}-C-phenylmethanesulfonamide
C-(4-Fluorophenyl)-N-{3-[3-hydroxy-4-(1,1,4-trioxo-1,2,5-thiadiazolidin-2-yl)-phenyl]-propyl}-methanesulfonamide
N-{3-[3-Hydroxy-4-(1,1,4-trioxo-1,2,5-thiadiazolidin-2-yl)-phenyl]-propyl}-4-isopropylbenzenesulfonamide
N-{3-[3-Hydroxy-4-(1,1,4)-trioxo-1,2,5-thiadiazolidin-2-yl)-phenyl]-propyl}-4-trifluoromethylbenzenesulfonamide
N-{3-[3-Hydroxy-4-(1,1,4-trioxo-1,2,5-thiadiazolidin-2-yl)-phenyl]-propyl}-4-trifluoromethoxybenzenesulfonamide
C-(3-Aminophenyl)-N-{3-[3-hydroxy-4-(1,1,4-trioxo-1,2,5-thiadiazolidin-2-yl)-phenyl]-propyl}-methanesulfonamide
N-{3-[3-Hydroxy-4-(1,1,4-trioxo-1,2,5-thiadiazolidin-2-yl)-phenyl]-propyl}-2,4,6-triisopropylbenzenesulfonamide
2-Hydroxy-5-{3-[3-hydroxy-4-(1,1,4-trioxo-1,2,5-thiadiazolidin-2-yl)-phenyl]-propylsulfamoyl}-benzoic acid
3-Amino-N-{3-[3-hydroxy-4-(1,1,4-trioxo-1,2,5-thiadiazolidin-2-yl)-phenyl]-propyl}-benzenesulfonamide
4-Amino-N-{3-[3-hydroxy-4-(1,1,4-trioxo-1,2,5-thiadiazolidin-2-yl)-phenyl]-propyl}-benzenesulfonamide
N-{3-[3-Hydroxy-4-(1,1,4-trioxo-1,2,5-thiadiazolidin-2-yl)-phenyl]-propyl}-3,5-dimethylbenzenesulfonamide
N-{3-[3-Hydroxy-4-(1,1,4-trioxo-1,2,5-thiadiazolidin-2-yl)-phenyl]-propyl}-2,5-dimethylbenzenesulfonamide
N-{3-[3-Hydroxy-4-(1,1,4-trioxo-1,2,5-thiadiazolidin-2-yl)-phenyl]-propyl}-2,4,6-trimethylbenzenesulfonamide
4-tert-Butyl-N-{3-[3-hydroxy-4-(1,1,4-trioxo-1,2,5-thiadiazolidin-2-yl)-phenyl]-propyl}-benzenesulfonamide
4-(1,1-Dimethylpropyl)-N-{3-[3-hydroxy-4-(1,1,4-trioxo-1,2,5-thiadiazolidin-2-yl)-phenyl]-propyl}-benzenesulfonamide
N-{3-[3-Hydroxy-4-(1,1,4-trioxo-1,2,5-thiadiazolidin-2-yl)-phenyl]-propyl}-3,4-dimethoxybenzenesulfonamide
N-{3-[3-Hydroxy-4-(1,1,4-trioxo-1,2,5-thiadiazolidin-2-yl)-phenyl]-propyl}-2,5-bis-(2,2,2-trifluoroethoxy)-benzenesulfonamide
Biphenyl-4-sulfonic acid {3-[3-hydroxy-4-(1,1,4-trioxo-1,2,5-thiadiazolidin-2-yl)-phenyl]-propyl}-amide
N-{3-[3-Hydroxy-4-(1,1,4-trioxo-1,2,5-thiadiazolidin-2-yl-phenyl]-propyl}-2-phenoxybenzenesulfonamide
N-{3-[3-Hydroxy-4-(1,1,4-trioxo-1,2,5-thiadiazolidin-2-yl)-phenyl]-propyl}-3-phenoxybenzenesulfonamide
N-{3-[3-Hydroxy-4-(1,1,4-trioxo-1,2,5-thiadiazolidin-2-yl)-phenyl]-propyl}-2,5-bis-(2,2,2-trifluoroethoxy)-benzenesulfonamide
2,2-Diphenylethanesulfonic acid {3-[3-hydroxy-4-(1,1,4-trioxo-1,2,5-thiadiazolidin-2-yl)-phenyl]-propyl}-amide
C-(2-Aminophenyl)-N{3-[3-hydroxy-4-(1,1,4-trioxo-1,2,5-thiadiazolidin-2-yl)-phenyl]-propyl}-methanesulfonamide
Naphthalene-1-sulfonic acid {3-[3-hydroxy-4-(1,1,4-trioxo-1,2,5-thiadiazolidin-2-yl)-phenyl]-propyl}-amide
C-Cyclohexyl-N-{3-[3-hydroxy-4-(1,1,4-trioxo-1,2,5-thiadiazolidin-2-yl)-phenyl]-propyl}-methanesulfonamide
2-Naphthalen-1-yl-ethanesulfonic acid {3-[3-hydroxy-4-(1,1,4-trioxo-1,2,5-thiadiazolidin-2-yl)-phenyl]-propyl}-amide
2-Phenyl-2-(2-trifluoromethylphenyl)-ethanesulfonic acid {3-[3-hydroxy-4-(1,1,4-trioxo-1,2,5-thiadiazolidin-2-yl)-phenyl]-propyl}-amide
2-Oxo-2H-chomene-6-sulfonic acid {3-[3-hydroxy-4-(1,1,4-trioxo-1,2,5-thiadiazolidin-2-yl)-phenyl]-propyl}-amide
N-{3-[3-Hydroxy-4-(1,1,4-trioxo-1,2,5-thiadiazolidin-2-yl)-phenyl]-propyl}-N-isopropylbenzenesulfonamide
N-(1-{2-[3-Hydroxy-4-(1,1,4-trioxo-1,2,5-thiadiazolidin-2-yl)-phenyl]-ethyl}-cyclopropyl)-benzenesulfonamide
N-{(S)-1-Benzyl-2-[3-hydroxy-4-(1,1,4-trioxo-1,2,5-thiadiazolidin-2-yl)-phenyl]-ethyl}-methanesulfonamide
Ethanesulfonic acid {(S)-1-benzyl-2-[3-hydroxy-4-(1,1,4-trioxo-1,2,5-thiadiazolidin-2-yl)-phenyl]-ethyl}-amide
N-{(S)-1-Benzyl-2-[3-hydroxy-4-(1,1,4-trioxo-1,2,5-thiadiazolidin-2-yl)-phenyl]-ethyl}-C-phenyl-methanesulfonamide
N-{(R)-1-Benzyl-2-[3-hydroxy-4-(1,1,4-trioxo-1,2,5-thiadiazolidin-2-yl)-phenyl]-ethyl}-C-phenyl methanesulfonamide
N-{4-[3-Hydroxy-4-(1,1,4-trioxo-1,2,5-thiadiazolidin-2-yl)-phenyl]-butyl}-methanesulfonamide
N-{5-[3-Hydroxy-4-(1,1,4-trioxo-1,2,5-thiadiazolidin-2-yl)-phenyl]-pentyl}-methanesulfonamide
5-[2-Hydroxy-4-(1-methanesulfonylpiperidin-3-ylmethyl)-phenyl]-1,1-dioxo-1,2,5-thiadiazolidin-3-one
5-{2-Hydroxy-4-[2-(1-methanesulfonyl piperidin-2-yl)-ethyl]-phenyl}-1,1-dioxo-1,2,5-thiadiazolidin-3-one
5-{4-[2-(1-Benzenesulfonylpiperidin-2-yl)-ethyl]-2-hydroxyphenyl}-1,1-dioxo-1,2,5-thiadiazolidin-3-one
5-{4-[2-((S)-1-Benzenesulfonylpiperidin-2-yl)-ethyl]-2-hydroxyphenyl}-1,1-dioxo-1,2,5-thiadiazolidin-3-one
5-{4-[2-((R)-1-Benzenesulfonylpiperidin-2-yl)-ethyl]-2-hydroxyphenyl}-1,1-dioxo-1,2,5-thiadiazolidin-3-one
5-{4-[2-(1-Benzenesulfonylpyrrolidin-2-yl)-ethyl]-2-hydroxyphenyl}-1,1-dioxo-1,2,5-thiadiazolidin-3-one
5-{4-[2-(1-Benzenesulfonyl-1H-pyrrol-2-yl)-ethyl]-2-hydroxyphenyl}-1,1-dioxo-1,2,5-thiadiazolidin-3-one
5-{4-[2-(1-Benzenesulfonylpyrrolidin-3-yl)-ethyl]-2-hydroxyphenyl}-1,1-dioxo-1,2,5-thiadiazolidin-3-one
5-{4-[2-(1-Benzenesulfonylazepan-2-yl)-ethyl]-2-hydroxyphenyl}-1,1-dioxo-1,2,5-thiadiazolidin-3-one
5-{2-Hydroxy-4-[2-((R)-2-methanesulfonyl-1,2,3,4-tetrahydroisoquinolin-3-yl)-ethyl]-phenyl}-1,1-dioxo-1,2,5-thiadiazolidin-3-one
5-{4-[2-((R)-2-Benzenesulfonyl-1,2,3,4-tetrahydroisoquinolin-3-yl)-ethyl]-2-hydroxyphenyl}-1,1-dioxo-1,2,5-thiadiazolidin-3-one
5-(2-Hydroxy-4-{2-[2-(4-trifluoromethylbenzenesulfonyl)-1,2,3,4-tetrahydroisoquinolin-3-yl]-ethyl}-phenyl)-1,1-dioxo-1,2,5-thiadiazolidin-3-one
5-{2-Hydroxy-4-[2-(2-phenylmethanesulfonyl-1,2,3,4-tetrahydroisoquinolin-3-yl)-ethyl]-phenyl}-1,1-dioxo-1,2,5-thiadiazolidin-3-one
5-{4-[2-(1,1-Dioxo-1,2-thiazinan-3-yl)-ethyl]-2-hydroxyphenyl}-1,1-dioxo-1,2,5-thiadiazolidin-3-one
N-{(1R*,2S*)-2-[3-Hydroxy-4-(1,1,4-trioxo-1,2,5-thiadiazolidin-2-yl)-benzyl]-cyclohexyl}-methanesulfonamide
N-{(1R,2S)-2-[3-Hydroxy-4-(1,1,4-trioxo-1,2,5-thiadiazolidin-2-yl)-benzyl]-cyclohexyl}-methanesulfonamide
N-{(1S,2R)-2-[3-Hydroxy-4-(1,1,4-trioxo-1,2,5-thiadiazolidin-2-yl)-benzyl]-cyclohexyl}-methanesulfonamide
Ethanesulfonic acid {(1R*,2S*)-2-[3-hydroxy-4-(1,1,4-trioxo-1,2,5-thiadiazolidin-2-yl)-benzyl]-cyclohexyl}-amide N-{(1R*,2S*)-2-[3-Hydroxy-4-(1,1,4-trioxo-1,2,5-thiadiazolidin-2-yl)-benzyl]-cyclohexyl}-benzenesulfonamide
(S)-2-Benzenesulfonylamino-3-[3-hydroxy-4-(1,1,4-trioxo-1,2,5-thiadiazolidin-2-yl)-phenyl]-N-pentyl propionamide
(S)-2-Benzenesulfonylamino-3-[3-hydroxy-4-(1,1,4-trioxo-1,2,5-thiadiazolidin-2-yl)-phenyl]-N-(4-phenylbutyl)-propionamide
N-{(S)-1-(1H-Benzoimidazol-2-yl)-2-[3-hydroxy-4-(1,1,4-trioxo-1,2,5-thiadiazolidin-2-yl)-phenyl]-ethyl}-benzenesulfonamide
tert-Butyl[({2-[4-(1,1-dioxido-4-oxo-1,2,5-thiadiazolidin-2-yl)-3-hydroxyphenyl]ethyl}amino)sulfonyl]carbamate
1-Cyclohexyl-3-{2-[3-hydroxy-4-(1,1,4-trioxo-1,2,5-thiadiazolidin-2-yl)-phenyl]-ethyl}-urea
1-{2-[3-Hydroxy-4-(1,1,4-trioxo-1,2,5-thiadiazolidin-2-yl)-phenyl]-ethyl}-3-phenyl-urea
1-Ethyl-3-{2-[3-hydroxy-4-(1,1,4-trioxo-1,2,5-thiadiazolidin-2-yl)-phenyl]-ethyl}-urea
1-Adamantan-1-yl-3-{2-[3-hydroxy-4-(1,1,4-trioxo-1,2,5-thiadiazolidin-2-yl)-phenyl]-ethyl}-urea
Benzenesulfonyl-N-{2-[3-hydroxy-4-(1,1,4-trioxo-1,2,5-thiadiazolidin-2-yl)-phenyl]-ethyl}-urea
1-(2,4-Dimethoxybenzyl)-3-{2-[3-hydroxy-4-(1,1,4-trioxo-1,2,5-thiadiazolidin-2-yl)-phenyl]-ethyl}-urea
1-(2-Hydroxyethyl)-3-{2-[3-hydroxy-4-(1,1,4-trioxo-1,2,5-thiadiazolidin-2-yl)-phenyl]-ethyl}-urea
3-{2-[3-Hydroxy-4-(1,1,4-trioxo-1,2,5-thiadiazolidin-2-yl)-phenyl]-ethyl}-1,1-bis-(2-methoxyethyl)-urea
Morpholine-4-carboxylic acid {2-[3-hydroxy-4-(1,1,4-trioxo-1,2,5-thiadiazolidin-2-yl)-phenyl]-ethyl}-amide
4-(3-{2-[3-Hydroxy-4-(1,1,4-trioxo-1,2,5-thiadiazolidin-2-yl)-phenyl]-ethyl}-ureido)-piperidine-1-carboxylic acid tert-butyl ester
1-{2-[3-Hydroxy-4-(1,1,4-trioxo-1,2,5-thiadiazolidin-2-yl)-phenyl]-ethyl}-3-piperidin-4-yl-urea
1-{3-[3-Hydroxy-4-(1,1,4-trioxo-1,2,5-thiadiazolidin-2-yl)-phenyl]-propyl}-3-phenyl-urea
1-Cyclohexyl-3-{3-[3-hydroxy-4-(1,1,4-trioxo-1,2,5-thiadiazolidin-2-yl)-phenyl]-propyl}-urea
1-Adamantan-1-yl-3-{3-[3-hydroxy-4-(1,1,4-trioxo-1,2,5-thiadiazolidin-2-yl)-phenyl]-propyl}-urea
3-{3-[3-Hydroxy-4-(1,1,4-trioxo-1,2,5-thiadiazolidin-2-yl)-phenyl]-propyl}-1H-quinazoline-2,4-dione
3-[3-Hydroxy-4-(1,1,4-trioxo-1,2,5-thiadiazolidin-2-yl)-benzyl]-piperidine-1-carboxylic acid ethylamide
5-(2-Hydroxy-4-methanesulfonylmethylphenyl)-1,1-dioxo-1,2,5-thiadiazolidin-3-one
5-(4-Ethanesulfonylmethyl-2-hydroxy-phenyl)-1,1-dioxo-1,2,5-thiadiazolidin-3-one
5-[2-Hydroxy-4-(propane-2-sulfonylmethyl)-phenyl]-1,1-dioxo-1,2,5-thiadiazolidin-3-one
5-(4-Benzenesulfonylmethyl-2-hydroxyphenyl)-1,1-dioxo-1,2,5-thiadiazolidin-3-one
5-(2-Hydroxy-4-methanesulfinylmethylphenyl)-1,1-dioxo-1,2,5-thiadiazolidin-3-one
5-(4-Ethanesulfinylmethyl-2-hydroxyphenyl)-1,1-dioxo-1,2,5-thiadiazolidin-3-one
5-[2-Hydroxy-4-(propane-2-sulfinylmethyl)-phenyl]-1,1-dioxo-1,2,5-thiadiazolidin-3-one
5-(2-Hydroxy-4-methylsulfanylmethylphenyl)-1,1-dioxo-1,2,5-thiadiazolidin-3-one
5-(4-Ethylsulfanylmethyl-2-hydroxyphenyl)-1,1-dioxo-1,2,5-thiadiazolidin-3-one
5-(2-Hydroxy-4-isopropylsulfanylmethylphenyl)-1,1-dioxo-1,2,5-thiadiazolidin-3-one
5-[4-(2-Benzenesulfonylethyl)-2-hydroxyphenyl]-1,1-dioxo-1,2,5-thiadiazolidin-3-one
5-[4-(4-Benzenesulfonylbutyl)-2-hydroxyphenyl]-1,1-dioxo-1,2,5-thiadiazolidin-3-one
5-{4-[3-(1,1-Dioxotetrahydrothiophen-2-yl)-prop-1-ynyl]-2-hydroxyphenyl}-1,1-dioxo-1,2,5-thiadiazolidin-3-one
5-{4-[3-(1,1-Dioxotetrahydrothiophen-2-yl)-propyl]-2-hydroxyphenyl}-1,1-dioxo-1,2,5-thiadiazolidin-3-one
5-[2-Hydroxy-4-(3-oxopentyl)-phenyl]-1,1-dioxo-1,2,5-thiadiazolidin-3-one
5-[2-Hydroxy-4-(2-methyl-3-oxopentyl)-phenyl]-1,1-dioxo-1,2,5-thiadiazolidin-3-one
5-[2-Hydroxy-4-(2-methyl-3-oxo-3-phenylpropyl)-phenyl]-1,1-dioxo-1,2,5-thiadiazolidin-3-one
5-[4-(2-Benzoylbutyl)-2-hydroxyphenyl]-1,1-dioxo-1,2,5-thiadiazolidin-3-one
5-[4-(2-Benzoylpentyl)-2-hydroxyphenyl]-1,1-dioxo-1,2,5-thiadiazolidin-3-one
5-[2-Hydroxy-4-(3-oxo-2,3-diphenylpropyl)-phenyl]-1,1-dioxo-1,2,5-thiadiazolidin-3-one
5-[4-(2-Benzyl-3-oxo-3-phenylpropyl)-2-hydroxyphenyl]-1,1-dioxo-1,2,5-thiadiazolidin-3-one
5-[4-(2,2-Dimethyl-3-oxo-3-phenylpropyl)-2-hydroxyphenyl]-1,1-dioxo-1,2,5-thiadiazolidin-3-one
5-[2-Hydroxy-4-(1-oxo-indan-2-ylmethyl)-phenyl]-1,1-dioxo-1,2,5-thiadiazolidin-3-one
5-[2-Hydroxy-4-(6-oxo-6,7,8,9-tetrahydro-5H-benzocyclohepten-5-ylmethyl)-phenyl]-1,1-dioxo-1,2,5-thiadiazolidin-3-one
5-[2-Hydroxy-4-(2-methoxy-3-oxo-3-phenylpropyl)-phenyl]-1,1-dioxo-1,2,5-thiadiazolidin-3-one
5-[2-Hydroxy-4-(3-hydroxy-2-methyl-3-phenylpropyl)-phenyl]-1,1-dioxo-1,2,5-thiadiazolidin-3-one
5-{2-Hydroxy-4-[2-(hydroxylphenylmethyl)-butyl]-phenyl}-1,1-dioxo-1,2,5-thiadiazolidin-3-one
5-{2-Hydroxy-4-[2-(hydroxyphenylmethyl)-pentyl]-phenyl}-1,1-dioxo-1,2,5-thiadiazolidin-3-one
5-[4-(2-Benzyl-3-hydroxy-3-phenylpropyl)-2-hydroxyphenyl]-1,1-dioxo-1,2,5-thiadiazolidin-3-one
5-[2-Hydroxy-4-(3-hydroxy-2,2-dimethyl-3-phenylpropyl)-phenyl]-1,1-dioxo-1,2,5-thiadiazolidin-3-one
5-[2-Hydroxy-4-(1-hydroxyindan-2-ylmethyl)-phenyl]-1,1-dioxo-1,2,5-thiadiazolidin-3-one
5-[2-Hydroxy-4-(3-hydroxy-2-methoxy-3-phenyl-propyl)-phenyl]-1,1-dioxo-1,2,5-thiadiazolidin-3-one
5-(2-Hydroxy-4-vinylphenyl)-1,1-dioxo-1,2,5-thiadiazolidin-3-one
5-[2-Hydroxy-4-(1-hydroxyethyl)-phenyl]-1,1-dioxo-1,2,5-thiadiazolidin-3-one
5-[2-Hydroxy-4-(2-hydroxyhexyl)-phenyl]-1,1-dioxo-1,2,5-thiadiazolidin-3-one
5-[2-Hydroxy-4-(3-hydroxybutyl)-phenyl]-1,1-dioxo-1,2,5-thiadiazolidin-3-one
5-{2-Hydroxy-4-[2-(1-hydroxycyclohexyl)-ethyl]-phenyl}-1,1-dioxo-1,2,5-thiadiazolidin-3-one
5-[2-Hydroxy-4-(4,4,4-trifluoro-3-hydroxy-3-phenylbutyl)-phenyl]-1,1-dioxo-1,2,5-thiadiazolidin-3-one
5-(3-Hydroxybiphenyl-4-yl)-1,1-dioxo-1,2,5-thiadiazolidin-3-one
5-(3,3′-Dihydroxybiphenyl-4-yl)-1,1-dioxo-1,2,5-thiadiazolidin-3-one
[3′-Hydroxy-4′-(1,1,4-trioxo-1,2,5-thiadiazolidin-2-yl)-biphenyl-4-yl]-acetic acid
5,5′-(3,3′-Dihydroxybiphenyl-4-yl)-1,1,1′,1′-tetraoxo-1,1′,2,2′,5,5′-dithiadiazolidin-3,3′-one
5-(4-Furan-3-yl-2-hydroxyphenyl)-1,1-dioxo-1,2,5-thiadiazolidin-3-one
5-(2-Hydroxy-4-thiophen-3-yl-phenyl)-1,1-dioxo-1,2,5-thiadiazolidin-3-one
5-(4-Benzofuran-3-yl-2-hydroxyphenyl)-1,1-dioxo-1,2,5-thiadiazolidin-3-one
5-[2-Hydroxy-4-(6-methoxybenzofuran-3-yl)-phenyl]-1,1-dioxo-1,2,5-thiadiazolidin-3-one
5-(2-Hydroxy-4-thiazol-5-yl-phenyl)-1,1-dioxo-1,2,5-thiadiazolidin-3-one
5-(2-Hydroxy-4-thiazol-2-yl-phenyl)-1,1-dioxo-1,2,5-thiadiazolidin-3-one
5-[2-Hydroxy-4-(1H-pyrrol-3-yl)-phenyl]-1,1-dioxo-1,2,5-thiadiazolidin-3-one
5-[2-Hydroxy-4-(1H-pyrazol-3-yl)-phenyl]-1,1-dioxo-1,2,5-thiadiazolidin-3-one
5-[2-Hydroxy-4-(1H-pyrazol-4-yl)-phenyl]-1,1-dioxo-1,2,5-thiadiazolidin-3-one
5-[2-Hydroxy-4-(1-propyl-1H-pyrazol-4-yl)-phenyl]-1,1-dioxo-1,2,5-thiadiazolidin-3-one
5-[2-Hydroxy-4-(1-isobutyl-1H-pyrazol-4-yl)-phenyl]-1,1-dioxo-1,2,5-thiadiazolidin-3-one
5-{2-Hydroxy-4-[1-(3-methylbutyl)-1H-pyrazol-4-yl]-phenyl}-1,1-dioxo-1,2,5-thiadiazolidin-3-one
5-[2-Hydroxy-4-(tetrahydrofuran-3-yl)-phenyl]-1,1-dioxo-1,2,5-thiadiazolidin-3-one
5-[4-(2,3-Dihydrobenzofuran-3-yl)-2-hydroxyphenyl]-1,1-dioxo-1,2,5-thiadiazolidin-3-one
5-(2-Hydroxy-4-thiazol-2-ylmethylphenyl)-1,1-dioxo-1,2,5-thiadiazolidin-3-one
5-[2-Hydroxy-4-(2H-pyrazol-3-ylmethyl)-phenyl]-1,1-dioxo-1,2,5-thiadiazolidin-3-one
5-(2-Hydroxy-4-pyrazol-1-ylmethyl-phenyl)-1,1-dioxo-1,2,5-thiadiazolidin-3-one
5-[2-Hydroxy-4-(3-trifluoromethylpyrazole-1-ylmethyl)-phenyl]-1,1-dioxo-1,2,5-thiadiazolidin-3-one
5-[3-Hydroxy-4-(1,1,4-trioxo-1,2,5-thiadiazolidin-2-yl)-phenyl]-pentanoic acid
4-[3-Hydroxy-4-(1,1,4-trioxo-1,2,5-thiadiazolidin-2-yl)-phenyl]-butane-1-sulfinic acid
4-[3-Hydroxy-4-(1,1,4-trioxo-1,2,5-thiadiazolidin-2-yl)-phenyl]-butyronitrile
4-[3-Hydroxy-4-(1,1,4-trioxo-1,2,5-thiadiazolidin-2-yl)-phenyl]-2-methyl-butyronitrile
4-[3-Hydroxy-4-(1,1,4-trioxo-1,2,5-thiadiazolidin-2-yl)-phenyl]-3,3-dimethylbutyronitrile
[3-Hydroxy-4-(1,1,4-trioxo-1,2,5-thiadiazolidin-2-yl)-phenoxy]-acetic acid 2-trimethylsilanylethyl ester
[3-Hydroxy-4-(1,1,4-trioxo-1,2,5-thiadiazolidin-2-yl)-phenoxy]-acetic acid
3-[3-Hydroxy-4-(1,1,4-trioxo-1,2,5-thiadiazolidin-2-yl)-phenoxy]-1,3,4,5-tetrahydro-benzo[b]azepin-2-one
5-(4-Ethyl-2-hydroxyphenyl)-1,1-dioxo-1,2,5-thiadiazolidin-3-one
5-(4-Hexyl-2-hydroxyphenyl)-1,1-dioxo-1,2,5-thiadiazolidin-3-one
5-(2-Hydroxy-4-isobutylphenyl)-1,1-dioxo-1,2,5-thiadiazolidin-3-one
5-[4-(3,3-Dimethylbutyl)-2-hydroxyphenyl]-1,1-dioxo-1,2,5-thiadiazolidin-3-one
5-[2-Hydroxy-4-(3,3,3-trifluoropropyl)-phenyl]-1,1-dioxo-1,2,5-thiadiazolidin-3-one
5-(4-Cyclopentylmethyl-2-hydroxyphenyl)-1,1-dioxo-1,2,5-thiadiazolidin-3-one
5-(4-Cyclohexylmethyl-2-hydroxyphenyl)-1,1-dioxo-1,2,5-thiadiazolidin-3-one
5-{2-Hydroxy-4-[1-(2,4,6-trimethylphenyl)-ethyl]-phenyl}-1,1-dioxo-1,2,5-thiadiazolidin-3-one
5-[4-(2-Aminobenzyl)-2-hydroxyphenyl]-1,1-dioxo-1,2,5-thiadiazolidin-3-one
5-[2-Hydroxy-4-(2-hydroxybenzyl)-phenyl]-1,1-dioxo-1,2,5-thiadiazolidin-3-one
5-[2-Hydroxy-4-(2-hydroxy-5-methylbenzyl)-phenyl]-1,1-dioxo-1,2,5-thiadiazolidin-3-one
5-[4-(2-Aminomethylbenzyl)-2-hydroxyphenyl]-1,1-dioxo-1,2,5-thiadiazolidin-3-one
5-[2-Hydroxy-4-(2-methoxymethylbenzyl)-phenyl]-1,1-dioxo-1,2,5-thiadiazolidin-3-one
{2-[3-Hydroxy-4-(1,1,4-trioxo-1,2,5-thiadiazolidin-2-yl)-benzyl]-phenyl}-acetonitrile
{2-[3-Hydroxy-4-(1,1,4-trioxo-1,2,5-thiadiazolidin-2-yl)-benzyl]-phenyl}-acetic acid methyl ester
{2-[3-Hydroxy-4-(1,1,4-trioxo-1,2,5-thiadiazolidin-2-yl)-benzyl]-phenyl}-acetic acid
N-Ethyl-2-{2-[3-hydroxy-4-(1,1,4-trioxo-1,2,5-thiadiazolidin-2-yl)-benzyl]-phenyl}-acetamide
5-(2-Hydroxy-4-{2-[2-(4-methylpiperidin-1-yl)-2-oxo-ethyl]-benzyl}-phenyl)-1,1-dioxo-1,2,5-thiadiazolidin-3-one
5-{2-Hydroxy-4-[2-(2-hydroxyethyl)-benzyl]-phenyl}-1,1-dioxo-1,2,5-thiadiazolidin-3-one
5-[2-Hydroxy-4-(pyridine-2-carbonyl)-phenyl]-1,1-dioxo-1,2,5-thiadiazolidin-3-one
5-(4-Benzenesulfonyl-2-hydroxyphenyl)-1,1-dioxo-1,2,5-thiadiazolidin-3-one
5-(2-Hydroxy-4-trifluoromethylphenyl)-1,1-dioxo-1,2,5-thiadiazolidin-3-one
5-(2-Hydroxy-4-methoxyphenyl)-1,1-dioxo-1,2,5-thiadiazolidin-3-one
3-Hydroxy-4-(1,1,4-trioxo-1,2,5-thiadiazolidin-2-yl)-benzonitrile, and
5-(4-Chloro-2-hydroxyphenyl)-1,1-dioxo-1,2,5-thiadiazolidin-3-one
   or a pharmaceutically acceptable salt thereof.

It will be understood that the invention has been described by way of example only and modifications may be made whilst remaining within the scope and spirit of the invention.

We claim:

1. A method of treating a musculoskeletal disease, comprising
identifying an individual exhibiting the musculoskeletal disease or at risk for developing the musculoskeletal disease; and
administering to the individual a therapeutically effective amount of a PTP inhibitor in combination with human growth hormone sufficient to alleviate the musculoskeletal disease.

2. (canceled)

3. The method of claim 1, wherein the musculoskeletal disease is muscle atrophy.

4. The method of claim 3, wherein the muscle atrophy is a result of treatment with a glucocorticoid.

5. The method of claim 4, wherein the glucocorticoid is cortisol, dexamethasone, betamethasone, prednisone, methylprednisolone, or prednisolone.

6. The method of claim 3, wherein the muscle atrophy is a result of denervation due to nerve trauma.

7. The method of claim 3, wherein the muscle atrophy is a result of degenerative, metabolic, or inflammatory neuropathy.

8. The method of claim 7, wherein the neuropathy is caused by Guillian-Barré syndrome, peripheral neuropathy, or exposure to environmental toxins or drugs.

9. The method of claim 3, wherein the muscle atrophy is a result of an adult motor neuron disease, infantile spinal muscular atrophy, juvenile spinal muscular atrophy, autoimmune motor neuropathy with multifocal conductor block, paralysis due to stroke or spinal cord injury, skeletal immobilization due to trauma, prolonged bed rest, voluntary inactivity, involuntary inactivity, metabolic stress, Of nutritional insufficiency, cancer, AIDS, fasting, rhabdomyolysis, a thyroid gland disorder, diabetes, benign congenital hypotonia, central core disease, nemalene myopathy, myotubular (centronuclear) myopathy, burn injury, chronic obstructive pulmonary disease, liver disease, sepsis, renal failure, congestive heart failure, or ageing.

10. The method of claim 1, wherein the musculoskeletal disease is a muscular dystrophy syndrome.

11. The method of claim 10, wherein the muscular dystrophy is Duchenne, Becker, myotonic, fascioscapulohumeral, Emery-Deifuss, oculopharyngeal, scapulohumeral, limb girdle, a congenital muscular dystrophy, or hereditary distal myopathy.

12. The method of claim 1, wherein the musculoskeletal disease is osteoporosis, a bone fracture, short stature, or dwarfism.

13. The method of claim 1 wherein the PTP inhibitor is a compound of the formula 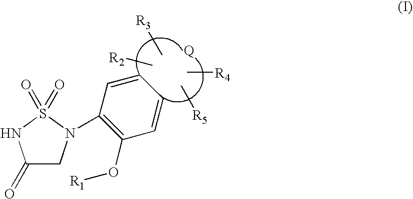
wherein
Q combined together with the carbon atoms to which it is attached form an aromatic, or a partially or fully saturated nonaromatic 5- to 8-membered carbocyclic or heterocyclic ring;
R₁ is hydrogen, —C(O)R₆, —C(O)NR₇R₉ or —C(O)OR₉ in which
R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂, R₃, R₄ and R₅ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, aryloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
R₂ and R₃ combined are alkylene which together with the ring atoms to which they are attached form a 3- to 7-membered fused ring; or
R₂ and R₃ combined are alkylene which together with the carbon atom to which they are attached form a 3- to 7-membered spirocyclic ring;
or a pharmaceutically acceptable salt thereof.

14. The method of claim 1, wherein the PTP inhibitor is a compound of the formula 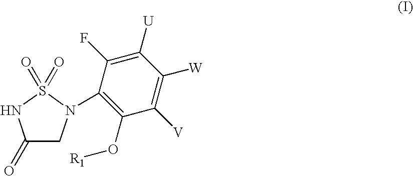
wherein
R₁ is hydrogen, —C(O)R₂, —C(O)NR₃R₄ or —C(O)OR₅ in which
R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
U, W and V are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, aryloxy, arylthio, heterocyclyl, heterocycloyloxy, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
U and W combined together with the carbon atoms to which they are attached form an optionally substituted aromatic, or a partially or fully saturated nonaromatic 5- to 8-membered carbocyclic or heterocyclic ring; or
W and V combined together with the carbon atoms to which they are attached form an optionally substituted aromatic, or partially or fully saturated nonaromatic 5- to 8-membered carbocyclic or heterocyclic ring;
or a pharmaceutically acceptable salt thereof.

15. The method of claim 1, wherein the PTP inhibitor is a compound of the formula 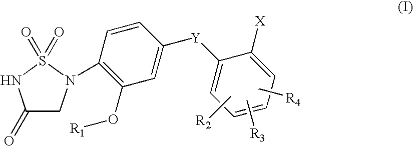
wherein
R₁ is hydrogen, —C(O)R₅, —C(O)NR₆R₇ or —C(O)OR₈ in which
R₅ and R₆ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₇ and R₈ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂, R₃ and R₄ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
R₂ and R₃ combined are alkylene which together with the ring atoms to which they are attached form a 5- to 7-membered fused ring provided R₂ and R₃ are attached to carbon atoms adjacent to each other; or
R₂ and R₃ combined together with the carbon atom to which they are attached form a fused 5- to 6-membered aromatic or heteroaromatic ring provided R₂ and R₃ are attached to carbon atoms adjacent to each other;
X is hydrogen, fluoro, cyano, or free or esterified carboxy; or
X is —NR₉C(O)R₁₀, —NR₉C(O)OR₁₁, —NR₉S(O)₂R₁₂, —(CH₂)ₘS(O)₂R₁₃, —OS(O)₂R₁₄ or —OₙC(O)NR₁₅R₁₆ in which
R₉ is hydrogen, lower alkyl, acyl, alkoxycarbonyl or sulfonyl;
R₁₀, R₁₁, R₁₂, R₁₃ and R₁₄ are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
R₁₀, R₁₂ and R₁₃ are, independently from each other, —NR₁₂R₁₈ in which
R₁₇ and R₁₈ are, independently from each other, hydrogen, alkyl, cycloalkyl, aralkyl, aryl or heterocyclyl; or
R₁₇ and R₁₈ combined are alkylene which together with the nitrogen atom to which they are attached form a 4- to 7-membered ring;
R₁₅ and R₁₆ are, independently from each other, hydrogen, alkyl, cycloalkyl, aralkyl, aryl or heterocyclyl; or
R₁₅ and R₁₆ combined are alkylene which together with the nitrogen atom to which they are attached form a 4- to 7-membered ring;
m and n are, independently from each other, zero or an integer of 1; or
C—X is replaced by nitrogen;
Y is CH₂, O or S;
or a pharmaceutically acceptable salt thereof.

16. The method of claim 1, wherein the PTP inhibitor is a compound of the formula 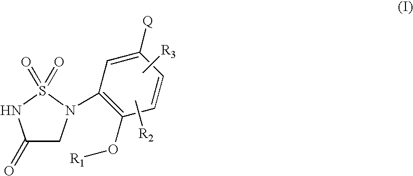
wherein
Q is alkoxy, alkylthio, alkylthiono, sulfonyl, cycloalkyl, aryl, aryloxy, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
R₁ is hydrogen, —C(O)R₄, —C(O)NR₅R₆ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂ and R₃ are, independently from each other, hydrogen, halogen, (C₁₋₃)alkyl or (C₁₋₃)alkoxy;
or a pharmaceutically acceptable salt thereof.

17. The method of claim 1, wherein the PTP inhibitor is a compound of the formula 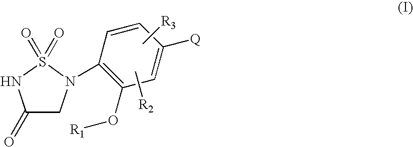
wherein
Q is:
i) —X, or
ii) —Y—(CH₂)ₙ—(CR₈R₉)ₚ—(CH₂)ₘ-Z-X in which;
Y is oxygen or S(O)_{q} in which q is zero or an integer of 1 or 2; or
Y is —C≡C— or —C═C—; or
Y is cyclopropyl or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, carbamoyl, aryl, or alkyl; or
R₈ and R₉ combined are alkylene which together with the carbon atom to which they are attached form a 3- to 7-membered ring;
p is zero or an integer selected from 1 or 2
Z is absent;
Z is —C(O)—O—; or
Z is —C(O)—; or
Z is —C(O)—NRα-alkylene- or —C(O)—NRα-alkylene-O—, wherein Rα is H or lower alkyl; or
Z is —CO—NRα-(CH₂)_{n′}(CR_{8′}R_{9′})_{p′}—(CH₂)_{m′}—, or —C(O)—NRα-(CH₂)_{n′}—(CR_{8′}R_{9′})_{p′}—(CH₂)_{m′}—O—, wherein p′ is zero or an integer of 1, n′ and m′ are, independently from each other, zero or an integer from 1 to 8, R_{8′} and R_{9′} are, independently from each other, hydrogen or lower alkyl, R□ is H or lower alkyl; or
Z is —NRα′-C(O)—, or —NRα′-C(O)—O—, wherein Rα′ is H or lower alkyl, or Rα′ and R₉ combined are alkylene which together with the carbon atom to which they are attached form a 3- to 7-membered ring; or
Z is —C(O)—NH—NH—C(O)—O—; or
Z is —S(O)₂—, or —S(O)—; or
Z is —NRβ-S(O)₂—, wherein Rβ is H, lower alkyl, or Rβ and R₉ combined are alkylene which together with the carbon atom to which they are attached form a 3- to 7-membered ring; or
Z is —NH—S(O)₂—NH—C(O)—O—; or
Z is —NRγ—C(O)—NRγ′—; wherein Rγ′ is H, alkyl, aryl, heterocyclyl, or lower alkoxy and Rγ is H, lower alkyl, or Rγ and R₉ combined are alkylene which together with the carbon atom to which they are attached form a 3- to 7-membered ring; or Rγ′ and X combined are alkylene which together with the carbon atom to which they are attached form a 3- to 7-membered ring or
Z is —NRτ-C(O)—NH—S(O)₂—, wherein Rτ is H or lower alkyl,
X is hydrogen, hydroxy, NH₂, halogen, alkoxy, alkylthio, alkyl, —S(O)—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
R₁ is hydrogen, —C(O)R₄, —C(O)NR₅R₆ or —C(O)OR₇ in which
R₄ and R₆ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₂ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂ and R₃ are, independently from each other, hydrogen, halogen, (C₁₋₃)alkyl or (C₁₋₃)alkoxy;
or a pharmaceutically acceptable salt thereof,
and wherein n+m+p is >1 or is 0, when X is aryl, and Y and Z are absent,
n+m+p is not 0 when X is —O-aryl, and Y and Z are absent, or
n+m+p is not 0 when X is —S-aryl, and Y and Z are absent or
n+m+p is not 0 when X is —CH₂-aryl, and Y and Z are absent, or
n+m+p is not 0 when X is aryl, Z is absent and Y is —O— or Y is —S—, or
wherein Q cannot be —CH₂-aryl, —S-aryl or —O-aryl.

18. The method of claim 1, further comprising administering an IGF1 molecule to the individual.

19. (canceled)

20. A method of increasing muscle or bone mass in an individual, the method comprising
identifying an individual in which increasing muscle or bone mass is desirable; and
administering to the individual an amount of a PTP inhibitor sufficient to increase the muscle or bone mass in the individual.

21. (canceled)

22. A pharmaceutical combination composition, comprising:
a protein tyrosine phosphatase inhibitor compound,
human growth hormone, and
one or more pharmaceutical excipients.

23. The composition according to claim 22, wherein the protein tyrosine phosphatase inhibitor compound and human growth hormone are a fixed single dosage composition.

24. The composition according to claim 22, wherein the protein tyrosine phosphatase inhibitor compound and human growth hormone are administered sequentially or concurrently.
